(19) United States
(12) Patent Application Publication
(10) Pub. No.: US 2026/0002219 A1
(43) Pub. Date: Jan. 1, 2026

(54) METHODS FOR DETECTION OF DISEASE

(71) Applicant: Universal Diagnostics, S.A., Seville (ES)

(72) Inventors: Kristi Kruusmaa, Ljubljana (SI); Pol Canal Noguer, Seville (ES); Vivian Erklavec Zajec, Ljubljana (SI); Francesco Mattia Mancuso, Seville (ES); Arne Willy Gouwy, Seville (ES); Miklós Gábriel Tulics, Seville (ES); Alejandro Requena Bermejo, Seville (ES); Pablo Pérez Martinez, Seville (ES); Carme Nolla Colomer, Seville (ES)

(21) Appl. No.: 19/180,595

(22) Filed: Apr. 16, 2025

Related U.S. Application Data
(60) Provisional application No. 63/636,343, filed on Apr. 19, 2024.
(60) Provisional application No. 63/691,058, filed on Sep. 5, 2024.

(51) Int. Cl. C12Q 1/6886; C12Q 1/6806; C12Q 1/6827; C12Q 1/6855; C12Q 1/6874

(52) U.S. Cl. CPC C12Q 1/6886; C12Q 1/6806; C12Q 1/6827; C12Q 1/6855; C12Q 1/6874; C12Q 2600/154

(57) ABSTRACT

The present disclosure provides for, among other things methods for identifying methylation statuses of markers (e.g., biomarkers). In various embodiments, the present disclosure provides methods for methods of detection (e.g., screening) of a disease or condition. A disease or condition discussed herein can be, e.g., advanced adenoma, colorectal cancer, other cancers, or other diseases or conditions associated with an aberrant methylation status. In various embodiments, the present disclosure provides methods for analysis of one or more methylation biomarkers in DNA (e.g., cell-free DNA, e.g., ctDNA) of a subject (e.g., a subject suspected of having colorectal cancer and/or advanced adenoma).

### CROSS-REFERENCE TO RELATED APPLICATIONS

This application claims priority to and the benefit of U.S. Provisional Application No. 63/691,058, filed Sep. 5, 2024, and U.S. Provisional Application No. 63/636,343, filed Apr. 19, 2024, the disclosures of which are incorporated by reference herein in their entireties.

### SEQUENCE LISTING

The instant application contains a Sequence Listing which has been submitted electronically in XML file format and is hereby incorporated by reference in its entirety. Said XML copy, created on Apr. 14, 2025, is named 2011722-0137_SL.xml and is 19,898 bytes in size.

### TECHNICAL FIELD

This invention relates generally to methods and systems for the detection of methylation status of markers (e.g., biomarkers). In various embodiments, the methods and systems described herein are used to detect methylation status of differentially-methylated regions (DMRs) of the human genome as markers.

### BACKGROUND

Disease detection is an important component of prevention of disease progression, diagnosis, and treatment. For example, early detection of colorectal cancer (CRC) has been shown to drastically improve outcomes of those suffering from CRC through early treatment of CRC. However, despite the availability of current tools to screen for and diagnose CRC and other cancers, millions of individuals still die annually from diseases, such as CRC, which are treatable through early intervention and detection. Current tools to screen for and diagnose diseases are insufficient. Accordingly, there is a need for tools and screening techniques to accurately screen for colorectal cancer at its earliest stages.

### SUMMARY

The present disclosure provides for, among other things, various methods and systems for identifying methylation status of markers (e.g., biomarkers). In certain embodiments, the methods and systems can be used in, for example, detection (e.g., screening) of a disease or condition. A disease or condition discussed herein can be, e.g., advanced adenoma, colorectal cancer, other cancers, or other diseases or conditions associated with an aberrant methylation status.

In various embodiments, the present disclosure provides methods for detecting colorectal cancer and/or advanced adenoma that include analysis of one or more methylation biomarkers in DNA (e.g., cell-free DNA, e.g., ctDNA) of a subject (e.g., a suspected of having colorectal cancer and/or advanced adenoma).

In one aspect, the invention is directed to a method (e.g., of detecting methylation statuses of one or more markers in a sample), the method comprising: detecting a methylation status for each of one or more markers identified in deoxyribonucleic acid (DNA) from a sample (e.g., a cell free DNA sample, cfDNA), wherein at least one of the one or more markers is a methylation locus comprising at least a portion of gene AC139713.2. In certain embodiments, each of the one or more markers is a methylation locus comprising at least a single differentially methylated region (DMR) or a portion of a DMR. In certain embodiments, at least one of the one or more markers is a methylation locus comprising at least a portion of SEQ ID NO. 12 (chr4: 143699944-143701144).

In another aspect, the invention is directed to a method (e.g., of detecting methylation statuses of one or more markers in a sample), the method comprising: detecting a methylation status for each of one or more markers identified in deoxyribonucleic acid (DNA) from a sample (e.g., a cell free DNA sample, cfDNA), wherein a first of the one or more markers is a methylation locus comprising at least a portion of gene LONRF2, a second of the one or more markers is a methylation locus comprising at least a portion of gene VAV3, and a third of the one or more markers is a methylation locus comprising at least a portion of gene MIR124-3. In certain embodiments, each of the one or more markers is a methylation locus comprising at least a single differentially methylated region (DMR) or a portion of a DMR. In certain embodiments, the first of the one or more markers is a methylation locus comprising at least a portion of SEQ ID NO. 11 (chr2: 100321258-100322771), the second of the one or more markers is a methylation locus comprising at least a portion of SEQ ID NO. 2 (chr1: 107963936-107966036), and the third of the one or more markers is a methylation locus comprising at least a portion of SEQ ID NO. 10 (chr20: 63177004-63178804). In certain embodiments, the one or more markers further comprises a fourth marker, wherein the fourth marker is a methylation locus comprising at least a portion of gene GATA5. In certain embodiments, the first of the one or more markers is a methylation locus comprising at least a portion of SEQ ID NO. 11 (chr2: 100321258-100322771), the second of the one or more markers is a methylation locus comprising at least a portion of SEQ ID NO. 2 (chr1: 107963936-107966036), the third of the one or more markers is a methylation locus comprising at least a portion of SEQ ID NO. 10 (chr20: 63177004-63178804), and the fourth of the one or more markers is a methylation locus comprising at least a portion of SEQ ID NO. 6 (chr20: 62476374-62476494).

In another aspect, the invention is directed to a method (e.g., of detecting methylation statuses of one or more markers), the method comprising: converting unmethylated cytosines of a plurality of DNA fragments in a sample (e.g., a cell free DNA sample, cfDNA) into uracils to generate a plurality of converted DNA fragments; sequencing the plurality of converted DNA fragments to generate a plurality of sequence reads, wherein each sequence read corresponds to a converted DNA fragment; and detecting a methylation status for each of one or more markers identified in the sequence reads, wherein at least one of the one or more markers is a methylation locus comprising at least a portion of gene AC139713.2. In certain embodiments, each of the one or more markers is a methylation locus comprising at least a single differentially methylated region (DMR) or a portion of a DMR. In certain embodiments, at least one of the one or more markers is a methylation locus comprising at least a portion of SEQ ID NO. 12 (chr4: 143699944-143701144).

In another aspect, the invention is directed to a method (e.g., of detecting methylation statuses of one or more markers), the method comprising: converting unmethylated cytosines of a plurality of DNA fragments in a sample (e.g., a cell free DNA sample, cfDNA) into uracils to generate a plurality of converted DNA fragments; sequencing the plurality of converted DNA fragments to generate a plurality of sequence reads, wherein each sequence read corresponds to a converted DNA fragment; and detecting a methylation status for each of one or more markers identified in the sequence reads, wherein each of the one or more markers is a methylation locus comprising at least a portion of a gene, wherein a first of the one markers is a methylation locus comprising at least a portion of gene LONRF2, a second of the one or more markers is a methylation locus comprising at least a portion of gene VAV3, and a third of the one or more markers is a methylation locus comprising at least a portion of gene MIR124-3. In certain embodiments, each of the one or more markers is a methylation locus comprising at least a single differentially methylated region (DMR) or a portion of a DMR. In certain embodiments, wherein the first of the one or more markers is a methylation locus comprising at least a portion of SEQ ID NO. 11 (chr2: 100321258-100322771), the second of the one or more markers is a methylation locus comprising at least a portion of SEQ ID NO. 2 (chr1: 107963936-107966036), and the third of the one or more markers is a methylation locus comprising at least a portion of SEQ ID NO. 10 (chr20: 63177004-63178804). In certain embodiments, the one or more markers further comprises a fourth marker, wherein the fourth marker is a methylation locus comprising at least a portion of gene GATA5. In certain embodiments, a first of the one or more markers is a methylation locus comprising at least a portion of SEQ ID NO. 11 (chr2: 100321258-100322771), a second of the one or more markers is a methylation locus comprising at least a portion of SEQ ID NO. 2 (chr1: 107963936-107966036), a third of the at least one or more markers is a methylation locus comprising at least a portion of SEQ ID NO. 10 (chr20: 63177004-63178804), and a fourth of the at least one or more markers is a methylation locus comprising at least a portion of SEQ ID NO. 6 (chr20: 62476374-62476494).

In certain embodiments, the sample is obtained from a subject (e.g., a human subject).

In certain embodiments, the method comprises automatically determining, by a processor of a computing device, a stage and/or a presence of colorectal cancer from the detected methylation status(es) of the one or more markers (e.g., automatically determining a likelihood of said stage and/or said presence of colorectal cancer). In certain embodiments, the step of automatically determining the stage and/or the presence of colorectal cancer is performed by the processor using a machine learning algorithm {e.g., a random forest (RF) machine learning algorithm}.

In one aspect, the invention is directed to a method (e.g., of detecting methylation statuses of one or more markers in a sample), the method comprising: detecting a methylation status for each of one or more markers (e.g., one, two, three, four, five, six, seven, eight, nine, ten, eleven, twelve, or thirteen markers) identified in deoxyribonucleic acid (DNA) from a sample (e.g., a cell free DNA sample, cfDNA), wherein each of the one or more markers is a methylation locus comprising at least a single differentially methylated region (DMR) or a portion of a DMR selected from the 13 DMRs listed below:

| Chromosome | | | | SEQ |
|---|---|---|---|---|
| No. | Start | End | Gene | ID NO. |
| 2 | 29920045 | 29921364 | ALK | 1 |
| 1 | 107963936 | 107966036 | VAV3 | 2 |
| 2 | 73292434 | 73292554 | EGR4 | 3 |
| 5 | 178590240 | 178590360 | COL23A1 | 4 |
| 5 | 173234839 | 173234959 | NKX2-5 | 5 |
| 20 | 62476374 | 62476494 | GATA5 | 6 |
| 8 | 72251358 | 72251490 | AC022905.1 | 7 |
| 2 | 236237696 | 236237816 | ASB18 | 8 |
| 6 | 105981552 | 105981672 | RN7SKP211 | 9 |
| 20 | 63177004 | 63178804 | MIR124-3 | 10 |
| 2 | 100321258 | 100322771 | LONRF2 | 11 |
| 4 | 143699944 | 143701144 | AC139713.2 | 12 |

In certain embodiments, detecting the methylation status comprises determining whether at least one methylation site within at least one of the one or more markers is hypermethylated or hypomethylated.

In certain embodiments, the subject is susceptible to colorectal cancer and/or advanced adenoma (e.g., differentiated as between CRC and AA or, e.g., undifferentiated as between CRC and AA). In certain embodiments, the subject is susceptible to stage III or stage IV colorectal cancer (e.g., differentiated and/or undifferentiated). In certain embodiments, the subject is susceptible to early stage (e.g., stage 0, stage I, stage II) colorectal cancer (e.g., differentiated and/or undifferentiated).

In certain embodiments, each methylation locus is equal to or less than 2200 bp in length (e.g., less than or equal to 2101 bp). In certain embodiments, each methylation locus is greater than or equal to 100 bp in length (e.g., greater than or equal to 121 bp).

In certain embodiments, the one or more markers comprises a methylation locus comprising at least a portion of SEQ ID NO. 12 (chr4: 143699944-143701144). In certain embodiments, the one or more markers comprises a methylation locus comprising at least a portion of SEQ ID NO. 1 (chr2: 29920045-29921364). In certain embodiments, the one or more markers comprises a methylation locus comprising at least a portion of SEQ ID NO. 11 (chr2: 100321258-100322771). In certain embodiments, a first of the one or more markers is a methylation locus comprising at least a portion of SEQ ID NO. 11 (chr2: 100321258-100322771) and a second of the one or more markers is a methylation locus comprising at least a portion of SEQ ID NO. 1 (chr2: 29920045-29921364). In certain embodiments, a first of the one or more markers is a methylation locus comprising at least a portion of SEQ ID NO. 10 (chr20: 63177004-63178804) and a second of the one or more markers is a methylation locus comprising at least a portion of SEQ ID NO. 11 (chr2: 100321258-100322771). In certain embodiments, a first of the one or more markers is a methylation locus comprising at least a portion of SEQ ID NO. 10 (chr20: 63177004-63178804) and a second of the one or more markers is a methylation locus comprising at least a portion SEQ ID NO. 2 (chr1: 107963936-107966036). In certain embodiments, a first of the one or more markers is a methylation locus comprising at least a portion of SEQ ID NO. 12 (chr4: 143699944-143701144) and a second of the one or more markers is a methylation locus comprising at least a portion SEQ ID NO. 2 (chr1: 107963936-107966036). In certain embodiments, a first of the one or more markers is a methylation locus comprising at least a portion of SEQ ID NO. 11 (chr2: 100321258-100322771), a second of the one or more markers is a methylation locus comprising at least a portion of SEQ ID NO. 2 (chr1: 107963936-107966036), and a third of the at least one of the one or more markers is a methylation locus comprising at least a portion of SEQ ID NO. 10 (chr20: 63177004-63178804). In certain embodiments, a first of the one or more markers is a methylation locus comprising at least a portion of SEQ ID NO. 11 (chr2: 100321258-100322771), a second of the one or more markers is a methylation locus comprising at least a portion of SEQ ID NO. 2 (chr1: 107963936-107966036), a third of the one or more markers is a methylation locus comprising at least a portion of SEQ ID NO. 10 (chr20: 63177004-63178804), and a fourth of the one or more markers is a methylation locus comprising at least a portion of SEQ ID NO. 6 (chr20: 62476374-62476494).

In certain embodiments, the sample is a member selected from the group consisting of a tissue sample, a blood sample, a stool sample, and a blood product sample. In certain embodiments, the sample comprises DNA (e.g., cfDNA) isolated from blood or plasma of the subject.

In certain embodiments, the method comprises isolating DNA (e.g., cfDNA) from at least 3 mL of plasma from the subject.

In certain embodiments, method comprises determining the methylation status of each of the one or more markers using next generation sequencing (NGS).

In certain embodiments, the sample comprises at least 8 ng, at least 10 ng, at least 15 ng, at least 20 ng or more of DNA.

In certain embodiments, the method comprises subjecting the DNA to an enzymatic treatment.

In certain embodiments, the sample is obtained from a subject (e.g., a human subject).

In another aspect, the invention is directed to a method (e.g., of detecting methylation statuses of one or more markers), the method comprising: converting unmethylated cytosines of a plurality of DNA fragments in a sample (e.g., a cell free DNA sample, cfDNA) into uracils to generate a plurality of converted DNA fragments; sequencing the plurality of converted DNA fragments to generate a plurality of sequence reads, wherein each sequence read corresponds to a converted DNA fragment; and detecting a methylation status for each of one or more markers (e.g., one, two, three, four, five, six, seven, eight, nine, ten, eleven, twelve, or thirteen markers) identified in the sequence reads, wherein each of the one or more markers is a methylation locus comprising at least a single differentially methylated region (DMR) or a portion of a DMR selected from the 13 DMRs listed below:

| Chromosome | | | | SEQ |
|---|---|---|---|---|
| No. | Start | End | Gene | ID NO. |
| 2 | 29920045 | 29921364 | ALK | 1 |
| 1 | 107963936 | 107966036 | VAV3 | 2 |
| 2 | 73292434 | 73292554 | EGR4 | 3 |
| 5 | 178590240 | 178590360 | COL23A1 | 4 |
| 5 | 173234839 | 173234959 | NKX2-5 | 5 |
| 20 | 62476374 | 62476494 | GATA5 | 6 |
| 8 | 72251358 | 72251490 | AC022905.1 | 7 |
| 2 | 236237696 | 236237816 | ASB18 | 8 |
| 6 | 105981552 | 105981672 | RN7SKP211 | 9 |
| 20 | 63177004 | 63178804 | MIR124-3 | 10 |
| 2 | 100321258 | 100322771 | LONRF2 | 11 |
| 4 | 143699944 | 143701144 | AC139713.2 | 12 |

In certain embodiments, converting the unmethylated cytosines of a plurality of DNA fragments in the sample into uracils comprises subjecting the plurality of DNA fragments to an enzymatic treatment.

In certain embodiments, the plurality of DNA fragments (in total) comprise at least 8 ng, at least 10 ng, at least 15 ng, at least 20 ng or more of DNA.

In certain embodiments, the method comprises isolating DNA (e.g., cfDNA) from at least 3 mL of plasma from the subject.

In certain embodiments, the method comprises adding one or more control DNA molecules (e.g., spike-in methylation conversion controls), wherein the sequence, number of methylated bases, and number of unmethylated bases of the control DNA molecules had been determined prior to addition of the control DNA to the sample.

In certain embodiments, the method comprises determining the number of unmethylated cytosines of the control DNA molecules that were converted into uracils.

In certain embodiments, the method comprises attaching (e.g., ligating) adapters to the plurality of DNA fragments. In certain embodiments, the adapter sequence is attached to the plurality of DNA fragments prior to conversion.

In certain embodiments, the method comprises amplifying the plurality of converted DNA fragments (e.g., a library prepared using converted DNA fragments). In certain embodiments, the method comprises amplifying the plurality of converted DNA fragments after attaching adapters to the plurality of DNA fragments.

In certain embodiments, the method comprises performing one or more quality control checks to determine the concentration and/or the ratios of fragments lengths of the amplified DNA fragments.

In certain embodiments, the method comprises using one or more capture baits that enrich for a target region to capture one or more corresponding methylation locus/loci. In certain embodiments, the capture baits comprise at least one capture probe that targets a fully methylated methylation locus. In certain embodiments, the capture baits comprise at least one capture probe that targets a fully unmethylated methylation locus. In certain embodiments, the capture baits comprise at least one capture probe that targets a partially methylated methylation locus. In certain embodiments, the capture baits comprise at least one capture probe that is fully methylated.

In certain embodiments, the method comprises capturing (e.g., hybridizing the one or more capture baits to) a subset of the DNA fragments using the one or more capture baits. In certain embodiments, the method comprises binding the captured subset of the DNA fragments to (e.g., indirectly to) a substrate (e.g complementary bait sequence). In certain embodiments, wherein the method comprises binding the captured subset of the DNA fragments to the substrate after amplification of the converted DNA fragments. In certain embodiments, the method comprises sequencing the plurality of converted DNA fragments at a read depth of at least 50×, at least 100×, at least 200×, at least 300×, at least 400×, at least 500×, at least 600×, at least 700×, at least 800×, at least 900×, at least 1000× or greater.

In certain embodiments, the method comprises mapping a subset of (e.g., up to all of) the plurality of sequence reads to a region of interest in a reference genome comprising at least one of the one or more markers (e.g., methylation markers, mutation markers).

In certain embodiments, the region of interest in the reference genome comprises at least 100, at least 200, at least 300, at least 400, at least 500, at least 600, at least 700, at least 800, at least 900, at least 1000 base pairs upstream and/or downstream of the at least one of the one or more markers (e.g., methylation markers, mutation markers).

In certain embodiments, the method comprises deduplicating the plurality of sequence reads generated from the plurality of converted DNA fragments. In certain embodiments, the method comprises deduplicating the plurality of sequence reads based on: (i) the start position of the sequence reads (i.e., the 5′ end coordinate); and/or (ii) the end position of the sequence reads (i.e., the 3′ end coordinate); and/or (iii) the methylation level of the sequence reads.

In certain embodiments, the method comprises removing, from the plurality of sequence reads, one or more poor-quality reads that failed one or more quality check criteria.

In certain embodiments, the method comprises detecting the presence or absence of one or more mutations based on sequence information from the plurality of sequence reads.

In certain embodiments, the one or more genomic mutations comprise a single nucleotide polymorphism, a deletion, an insertion, or a combination thereof. In certain embodiments, the one or more genomic mutations comprise a single nucleotide variant (e.g., a single nucleotide polymorphism), an inversion, a deletion, an insertion, a transversion, a translocation, a fusion, a truncation, an amplification, or a combination thereof.

In certain embodiments, the methylation status is a read-wise methylation value.

In certain embodiments, the sample is obtained from a subject (e.g., a human subject).

In another aspect, the invention is directed to a method (e.g., of detecting methylation statuses of one or more markers in a sample), the method comprising: detecting a methylation status for each of one or more markers identified in deoxyribonucleic acid (DNA) from a sample, wherein each of the one or more markers is a methylation locus comprising at least a portion of a gene selected from the 12 genes listed below:

ALK
VAV3
EGR4
COL23A1
NKX2-5
GATA5
AC022905.1
ASB18
RN7SKP211
MIR124-3
LONRF2
AC139713.2

In certain embodiments, a first of the one or more markers is a methylation locus comprising at least a portion of gene LONRF2 and a second of the one or more markers is a methylation locus comprising at least a portion of gene ALK. In certain embodiments, a first of the one or more markers is a methylation locus comprising at least a portion of gene VAV3. In certain embodiments, a second of the one or more markers is a methylation locus comprising at least a portion of gene MIR124-3. In certain embodiments, a second of the one or more markers is a methylation locus comprising at least a portion of gene AC139713.2. In certain embodiments, a first of the one or more markers is a methylation locus comprising at least a portion of gene VAV3, a second of the one or more markers is a methylation locus comprising at least a portion of gene MIR124-3, and a third of the one or more markers is a methylation locus comprising at least a portion of gene LONRF2. In certain embodiments, a first of the one or more markers is a methylation locus comprising at least a portion of gene VAV3, a second of the one or more markers is a methylation locus comprising at least a portion of gene GATA5, a third of the one or more markers is a methylation locus comprising at least a portion of gene MIR124-3, and a fourth of the one or more markers is a methylation locus comprising at least a portion of gene LONRF2. In certain embodiments, a fifth of the one or more markers is a methylation locus comprising at least a portion of gene AC022905.1 and a sixth of the one or more markers is a methylation locus comprising at least a portion of gene AC139713.2. In certain embodiments, a first of the one or more markers is a methylation locus comprising at least a portion of gene MIR124-3 and a second of the one or more markers is a methylation locus comprising at least a portion of gene LONRF2.

In certain embodiments, the subject is susceptible to colorectal cancer and/or advanced adenoma. In certain embodiments, the subject is susceptible to stage II to stage IV (e.g., stage III, stage IV) colorectal cancer. In certain embodiments, the subject is susceptible to early stage (e.g., stage 0, stage I, stage II) colorectal cancer.

In certain embodiments, the sample is a member selected from the group consisting of a tissue sample, a blood sample, a stool sample, and a blood product sample. In certain embodiments, the sample is obtained from a subject (e.g., a human subject). In certain embodiments, the sample comprises DNA isolated from blood or plasma of the subject.

In certain embodiments, each sequence read overlaps with a portion of at least one of the one or more markers.

In certain embodiments, the overlapping portion of each sequence read and the at least one of the one or more markers comprises at least 2 (e.g., at least 3, at least 4, at least 5, at least 6, at least 7, at least 8 or more) CpGs. In certain embodiments, the overlapping portion of each sequence read and the at least one of the one or more markers comprises 27 or fewer (e.g., 26 or fewer, 25 or fewer, 20 or fewer, 17 or fewer) CpGs.

In certain embodiments, detecting the methylation status for each of one or more markers identified in the sequence reads comprises identifying, in the overlapping portion of each sequence read and the at least one of the one or more markers, a number of sequence reads having at least 50% (e.g., at least 60%, at least 70%, at least 75%, at least 80%, at least 90%, or all) of the CpGs methylated in the overlapping portion.

In another aspect, the invention is directed to a method of detecting methylation statuses of one or more markers, the method comprising: converting unmethylated cytosines of a plurality of DNA fragments in a sample into uracils to generate a plurality of converted DNA fragments, wherein the plurality of DNA fragments were obtained from a sample; sequencing the plurality of converted DNA fragments to generate a plurality of sequence reads, wherein each sequence read corresponds to a converted DNA fragment; and detecting a methylation status for each of one or more markers identified in the sequence reads, wherein each of the one or more markers is a methylation locus comprising at least a portion of a gene selected from the 12 genes listed below:

ALK
VAV3
EGR4
COL23A1
NKX2-5
GATA5
AC022905.1
ASB18
RN7SKP211
MIR124-3
LONRF2
AC139713.2

In certain embodiments, the one or more markers is a methylation locus comprising at least a portion of gene AC139713.2. In certain embodiments, a first of the one or more markers is a methylation locus comprising at least a portion of gene LONRF2, a second of the one or more markers is a methylation locus comprising at least a portion of gene VAV3, and a third of the one or more markers is a methylation locus comprising at least a portion of gene MIR124-3. In certain embodiments, a fourth of the one or more markers is a methylation locus comprising at least a portion of gene GATA5.

In certain embodiments, the sample is obtained from a subject (e.g., a human subject).

In another aspect, the invention is directed to a method (e.g., of detecting methylation statuses of one or more markers, e.g., in a DMR or gene listed in Table 1), the method comprising: converting unmethylated cytosines of a plurality of DNA fragments in a sample (e.g., a cell free DNA sample, cfDNA) obtained from a subject (e.g., a human subject) into uracils to generate a plurality of converted DNA fragments; sequencing the plurality of converted DNA fragments to generate a plurality of sequence reads, wherein each sequence read corresponds to a converted DNA fragment; and detecting a methylation status for each of one or more markers (e.g., one, two, three, four, five, six, seven, eight, nine, ten, eleven, twelve, or thirteen markers) identified in the sequence reads, wherein each of the one or more markers is a methylation locus comprising at least a single differentially methylated region (DMR) or a portion of a DMR.

In certain embodiments, converting the unmethylated cytosines of a plurality of DNA fragments in the sample into uracils comprises subjecting the plurality of DNA fragments to an enzymatic treatment. In certain embodiments, the plurality of DNA fragments (in total) comprise at least 8 ng, at least 10 ng, at least 15 ng, at least 20 ng or more of DNA.

In certain embodiments, the method comprises isolating DNA (e.g., cfDNA) from at least 3 mL of plasma from the subject.

In certain embodiments, the method comprises adding one or more control DNA molecules (e.g., spike-in methylation conversion controls), wherein the sequence, number of methylated bases, and number of unmethylated bases of the control DNA molecules had been determined prior to addition of the control DNA to the sample.

In certain embodiments, the method comprises determining the number of unmethylated cytosines of the control DNA molecules that were converted into uracils.

In certain embodiments, the method comprises attaching (e.g., ligating) adapters to the plurality of DNA fragments.

In certain embodiments, the adapter sequence is attached to the plurality of DNA fragments prior to conversion.

In certain embodiments, the method comprises amplifying the plurality of converted DNA fragments (e.g., a library prepared using converted DNA fragments). In certain embodiments, the method comprises amplifying the plurality of converted DNA fragments after attaching adapters to the plurality of DNA fragments.

In certain embodiments, the method comprises performing one or more quality control checks to determine the concentration and/or the ratios of fragments lengths of the amplified DNA fragments.

In certain embodiments, the method comprises using one or more capture baits that enrich for a target region to capture one or more corresponding methylation locus/loci. In certain embodiments, the capture baits comprise at least one capture probe that targets a fully methylated methylation locus. In certain embodiments, the capture baits comprise at least one capture probe that targets a fully unmethylated methylation locus. In certain embodiments, the capture baits comprise at least one capture probe that targets a partially methylated methylation locus. In certain embodiments, the capture baits comprise at least one capture probe that is fully methylated.

In certain embodiments, the method comprises capturing (e.g., hybridizing the one or more capture baits to) a subset of the DNA fragments using the one or more capture baits.

In certain embodiments, the method comprises binding the captured subset of the DNA fragments to (e.g., indirectly to) a substrate (e.g complementary bait sequence).

In certain embodiments, the method comprises binding the captured subset of the DNA fragments to the substrate after amplification of the converted DNA fragments.

In certain embodiments, the method comprises sequencing the plurality of converted DNA fragments at a read depth of at least 50×, at least 100×, at least 200×, at least 300×, at least 400×, at least 500×, at least 600×, at least 700×, at least 800×, at least 900×, at least 1000× or greater.

In certain embodiments, the method comprises mapping a subset of (e.g., up to all of) the plurality of sequence reads to a region of interest in a reference genome comprising at least one of the one or more markers (e.g., methylation markers, mutation markers).

In certain embodiments, the region of interest in the reference genome comprises at least 100, at least 200, at least 300, at least 400, at least 500, at least 600, at least 700, at least 800, at least 900, at least 1000 base pairs upstream and/or downstream of the at least one of the one or more markers (e.g., methylation markers, mutation markers).

In certain embodiments, the method comprises deduplicating the plurality of sequence reads generated from the plurality of converted DNA fragments.

In certain embodiments, the method comprises deduplicating the plurality of sequence reads based on: (i) the start position of the sequence reads (i.e., the 5′ end coordinate); and/or (ii) the end position of the sequence reads (i.e., the 3′ end coordinate); and/or (iii) the methylation level of the sequence reads.

In certain embodiments, the method comprises removing, from the plurality of sequence reads, one or more poor-quality reads that failed one or more quality check criteria.

In certain embodiments, the method comprises detecting the presence or absence of one or more mutations based on sequence information from the plurality of sequence reads.

In certain embodiments, the one or more genomic mutations comprise a single nucleotide polymorphism, a deletion, an insertion, or a combination thereof.

In certain embodiments, the one or more genomic mutations comprise a single nucleotide variant (e.g., a single nucleotide polymorphism), an inversion, a deletion, an insertion, a transversion, a translocation, a fusion, a truncation, an amplification, or a combination thereof.

In certain embodiments, the method comprises automatically determining, by a processor of a computing device, a stage and/or a presence of colorectal cancer from the detected methylation status(es) of the one or more markers (e.g., automatically determining a likelihood of said stage and/or said presence of colorectal cancer). In certain embodiments, automatically determining the stage and/or the presence of colorectal cancer is performed by the processor using a machine learning algorithm (e.g., a supervised deep learning algorithm, e.g., a deep neural network). In certain embodiments, the machine learning algorithm comprises a random forest (RF) machine learning algorithm.

In other aspects, the invention is directed to a system for performing any of the methods referred to in the preceding paragraphs, the system comprising a processor; and a memory having instructions thereon, the instructions, when executed by the processor, causing the processor to perform one or more (up to all) steps of the method.

In other aspects, the invention is directed to methods for detection (e.g., screening) of a disease or condition using any of the methods referred to in the preceding paragraphs. A disease or condition discussed herein can be, e.g., advanced adenoma, colorectal cancer, other cancers, or other diseases or conditions associated with an aberrant methylation status.

### Definitions

A or An: The articles “a” and “an” are used herein to refer to one or to more than one (i.e., to at least one) of the grammatical object of the article. By way of example, “an element” refers to one element or more than one element.

About: The term “about”, when used herein in reference to a value, refers to a value that is similar, in context, to the referenced value. In general, those skilled in the art, familiar with the context, will appreciate the relevant degree of variance encompassed by “about” in that context. For example, in some embodiments, e.g., as set forth herein, the term “about” can encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or with a fraction of a percent, of the referred value.

Advanced Adenoma: As used herein, the term “advanced adenoma” typically refers to refer to cells that exhibit first indications of relatively abnormal, uncontrolled, and/or autonomous growth but are not yet classified as cancerous alterations. In the context of colon tissue, “advanced adenoma” refers to neoplastic growth that shows signs of high grade dysplasia, and/or size that is >=10 mm, and/or villous histological type, and/or serrated histological type with any type of dysplasia.

Administration: As used herein, the term “administration” typically refers to the administration of a composition to a subject or system, for example to achieve delivery of an agent that is, is included in, or is otherwise delivered by, the composition.

Agent: As used herein, the term “agent” refers to an entity (e.g., for example, a small molecule, peptide, polypeptide, nucleic acid, lipid, polysaccharide, complex, combination, mixture, system, or phenomenon such as heat, electric current, electric field, magnetic force, magnetic field, etc.).

Amelioration: As used herein, the term “amelioration” refers to the prevention, reduction, palliation, or improvement of a state of a subject. Amelioration includes, but does not require, complete recovery or complete prevention of a disease, disorder or condition.

Biological Sample: As used herein, the term “biological sample” typically refers to a sample obtained or derived from a biological source (e.g., a tissue or organism or cell culture) of interest, as described herein. In some embodiments, e.g., as set forth herein, a biological source is or includes an organism, such as an animal or human. In some embodiments, e.g., as set forth herein, a biological sample is or include biological tissue or fluid. In some embodiments, e.g., as set forth herein, a biological sample can be or include cells, tissue, or bodily fluid. In some embodiments, e.g., as set forth herein, a biological sample can be or include blood, blood cells, cell-free DNA, free floating nucleic acids, ascites, biopsy samples, surgical specimens, cell-containing body fluids, sputum, saliva, feces, urine, cerebrospinal fluid, peritoneal fluid, pleural fluid, lymph, gynecological fluids, secretions, excretions, skin swabs, vaginal swabs, oral swabs, nasal swabs, washings or lavages such as a ductal lavages or broncheoalveolar lavages, aspirates, scrapings, bone marrow. In some embodiments, e.g., as set forth herein, a biological sample is or includes cells obtained from a single subject or from a plurality of subjects. A sample can be a “primary sample” obtained directly from a biological source, or can be a “processed sample.” A biological sample can also be referred to as a “sample.”

Biomarker: As used herein, the term “biomarker,” consistent with its use in the art, refers to a to an entity whose presence, level, or form, correlates with a particular biological event or state of interest, so that it is considered to be a “marker” of that event or state. Those of skill in the art will appreciate, for instance, in the context of a DNA biomarker, that a biomarker can be or include a locus (such as one or more methylation loci) and/or the status of a locus (e.g., the status of one or more methylation loci). To give but a few examples of biomarkers, in some embodiments, e.g., as set forth herein, a biomarker can be or include a marker for a particular disease, disorder or condition, or can be a marker for qualitative of quantitative probability that a particular disease, disorder or condition can develop, occur, or reoccur, e.g., in a subject. In some embodiments, e.g., as set forth herein, a biomarker can be or include a marker for a particular therapeutic outcome, or qualitative of quantitative probability thereof. Thus, in various embodiments, e.g., as set forth herein, a biomarker can be predictive, prognostic, and/or diagnostic, of the relevant biological event or state of interest. A biomarker can be an entity of any chemical class. For example, in some embodiments, e.g., as set forth herein, a biomarker can be or include a nucleic acid, a polypeptide, a lipid, a carbohydrate, a small molecule, an inorganic agent (e.g., a metal or ion), or a combination thereof. In some embodiments, e.g., as set forth herein, a biomarker is a cell surface marker. In some embodiments, e.g., as set forth herein, a biomarker is intracellular. In some embodiments, e.g., as set forth herein, a biomarker is found outside of cells (e.g., is secreted or is otherwise generated or present outside of cells, e.g., in a body fluid such as blood, urine, tears, saliva, cerebrospinal fluid, and the like). In some embodiments, e.g., as set forth herein, a biomarker is methylation status of a methylation locus. In some instances, e.g., as set forth herein, a biomarker may be referred to as a “marker.”

To give but one example of a biomarker, in some embodiments e.g., as set forth herein, the term refers to expression of a product encoded by a gene, expression of which is characteristic of a particular tumor, tumor subclass, stage of tumor, etc. Alternatively or additionally, in some embodiments, e.g., as set forth herein, presence or level of a particular marker can correlate with activity (or activity level) of a particular signaling pathway, for example, of a signaling pathway the activity of which is characteristic of a particular class of tumors.

Those of skill in the art will appreciate that a biomarker may be individually determinative of a particular biological event or state of interest, or may represent or contribute to a determination of the statistical probability of a particular biological event or state of interest. Those of skill in the art will appreciate that markers may differ in their specificity and/or sensitivity as related to a particular biological event or state of interest.

Blood component: As used herein, the term “blood component” refers to any component of whole blood, including red blood cells, white blood cells, plasma, platelets, endothelial cells, mesothelial cells, epithelial cells, and cell-free DNA. Blood components also include the components of plasma, including proteins, metabolites, lipids, nucleic acids, and carbohydrates, and any other cells that can be present in blood, e.g., due to pregnancy, organ transplant, infection, injury, or disease.

Cancer: As used herein, the terms “cancer,” “malignancy,” “neoplasm,” “tumor,” and “carcinoma,” are used interchangeably to refer to a disease, disorder, or condition in which cells exhibit or exhibited relatively abnormal, uncontrolled, and/or autonomous growth, so that they display or displayed an abnormally elevated proliferation rate and/or aberrant growth phenotype. In some embodiments, e.g., as set forth herein, a cancer can include one or more tumors. In some embodiments e.g., as set forth herein, a cancer can be or include cells that are precancerous (e.g., benign), malignant, pre-metastatic, metastatic, and/or non-metastatic. In some embodiments e.g., as set forth herein, a cancer can be or include a solid tumor. In some embodiments e.g., as set forth herein, a cancer can be or include a hematologic tumor. In general, examples of different types of cancers known in the art include, for example, colorectal cancer, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

Comparable: As used herein, the term “comparable” refers to members within sets of two or more conditions, circumstances, agents, entities, populations, etc., that may not be identical to one another but that are sufficiently similar to permit comparison there between, such that one of skill in the art will appreciate that conclusions can reasonably be drawn based on differences or similarities observed. In some embodiments, e.g., as sort forth herein, comparable sets of conditions, circumstances, agents, entities, populations, etc. are typically characterized by a plurality of substantially identical features and zero, one, or a plurality of differing features. Those of ordinary skill in the art will understand, in context, what degree of identity is required to render members of a set comparable. For example, those of ordinary skill in the art will appreciate that members of sets of conditions, circumstances, agents, entities, populations, etc., are comparable to one another when characterized by a sufficient number and type of substantially identical features to warrant a reasonable conclusion that differences observed can be attributed in whole or part to non-identical features thereof.

Corresponding to: As used herein, the term “corresponding to” refers to a relationship between two or more entities. For example, the term “corresponding to” may be used to designate the position/identity of a structural element in a compound or composition relative to another compound or composition (e.g., to an appropriate reference compound or composition). For example, in some embodiments, a monomeric residue in a polymer (e.g., a nucleic acid residue in a polynucleotide) may be identified as “corresponding to” a residue in an appropriate reference polymer. Those of ordinary skill in the art readily appreciate how to identify “corresponding” nucleic acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in nucleic acids in accordance with the present disclosure. Those of skill in the art will also appreciate that, in some instances, the term “corresponding to” may be used to describe an event or entity that shares a relevant similarity with another event or entity (e.g., an appropriate reference event or entity). To give but one example, a fragment of DNA in a sample from a subject may be described as “corresponding to” a gene in order to indicate, in some embodiments, that it shows a particular degree of sequence identity or homology, or shares a particular characteristic sequence element.

Detectable moiety: The term “detectable moiety” as used herein refers to any element, molecule, functional group, compound, fragment, or other moiety that is detectable. In some embodiments, e.g., as sort forth herein, a detectable moiety is provided or utilized alone. In some embodiments, e.g., as sort forth herein, a detectable moiety is provided and/or utilized in association with (e.g., joined to) another agent. Examples of detectable moieties include, but are not limited to, various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes, chemiluminescent agents, bioluminescent agents, spectrally resolvable inorganic fluorescent semiconductors nanocrystals (i.e., quantum dots), metal nanoparticles, nanoclusters, paramagnetic metal ions, enzymes, colorimetric labels, biotin, dioxigenin, haptens, and proteins for which antisera or monoclonal antibodies are available.

Diagnosis: As used herein, the term “Diagnosis” refers to determining whether, and/or the qualitative of quantitative probability that, a subject has or will develop a disease, disorder, condition, or state. For example, in diagnosis of cancer, diagnosis can include a determination regarding the risk, type, stage, malignancy, or other classification of a cancer. In some instances, e.g., as sort forth herein, a diagnosis can be or include a determination relating to prognosis and/or likely response to one or more general or particular therapeutic agents or regimens.

Diagnostic information: As used herein, the term “diagnostic information” refers to information useful in providing a diagnosis. Diagnostic information can include, without limitation, biomarker status information.

Differentially methylated: As used herein, the term “differentially methylated” describes a methylation site for which the methylation status differs between a first condition and a second condition. A methylation site that is differentially methylated can be referred to as a differentially methylated site. In some instances, e.g., as sort forth herein, a DMR is defined by the amplicon produced by amplification using oligonucleotide primers, e.g., a pair of oligonucleotide primers selected for amplification of the DMR or for amplification of a DNA region of interest present in the amplicon. In some instances, e.g., as sort forth herein, a DMR is defined as a DNA region amplified by a pair of oligonucleotide primers, including the region having the sequence of, or a sequence complementary to, the oligonucleotide primers. In some instances, e.g., as sort forth herein, a DMR is defined as a DNA region amplified by a pair of oligonucleotide primers, excluding the region having the sequence of, or a sequence complementary to, the oligonucleotide primers. As used herein, a specifically provided DMR can be unambiguously identified by the name of an associated gene followed by three digits of a starting position, such that, for example, a DMR starting at position 100785927 of ZAN can be identified as ZAN '927. As used herein, a specifically provided DMR can be unambiguously identified by the chromosome number followed by the starting and ending positions of a DMR. In certain embodiments, the start and end positions provided is given based on a 1-based reference, and the start and end position of the region are inclusive.

Differentially methylated region: As used herein, the term “differentially methylated region” (DMR) refers to a DNA region that includes one or more differentially methylated sites. A DMR that includes a greater number or frequency of methylated sites under a selected condition of interest, such as a cancerous state, can be referred to as a hypermethylated DMR. A DMR that includes a smaller number or frequency of methylated sites under a selected condition of interest, such as a cancerous state, can be referred to as a hypomethylated DMR. A DMR that is a methylation biomarker for colorectal cancer can be referred to as a colorectal cancer DMR. A DMR that is a methylation biomarker for advanced adenoma can be referred to as an advanced adenoma DMR. In some instances, e.g., as set forth herein, a DMR can be a single nucleotide, which single nucleotide is a methylation site. In some instances, e.g., as set forth herein, a DMR has a length of at least 10, at least 15, at least 20, at least 30, at least 50, or at least 75, at least 100, at least 121 base pairs. In some instances, e.g., as set forth herein, a DMR has a length of equal to or less than 5,000 bp, 4,000 bp, 3,000 bp, 2,000 bp, 2,200 bp, 2,101 bp, 1,000 bp, 950 bp, 900 bp, 850 bp, 800 bp, 750 bp, 700 bp, 650 bp, 600 bp, 550 bp, 500 bp, 450 bp, 400 bp, 350 bp, 300 bp, 250 bp, 200 bp, 150 bp, 100 bp, 50 bp, 40 bp, 30 bp, 20 bp, or 10 bp (e.g., where methylation status is determined using quantitative polymerase chain reaction (qPCR), e.g., methylation sensitive restriction enzyme quantitative polymerase chain reaction (MSRE-qPCR)) (e.g., where methylation status is determined using a next generation sequencing technique, e.g., targeted next generation sequencing). In some instances, e.g., as set forth herein, a DMR that is a methylation biomarker for advanced adenoma may also be useful in identification of colorectal cancer and vice versa.

DNA region: As used herein, “DNA region” refers to any contiguous portion of a larger DNA molecule. Those of skill in the art will be familiar with techniques for determining whether a first DNA region and a second DNA region correspond, based, e.g., on sequence similarity (e.g., sequence identity or homology) of the first and second DNA regions and/or context (e.g., the sequence identity or homology of nucleic acids upstream and/or downstream of the first and second DNA regions).

Except as otherwise specified herein, sequences found in or relating to humans (e.g., that hybridize to human DNA) are found in, based on, and/or derived from the example representative human genome sequence commonly referred to, and known to those of skill in the art, as Homo sapiens (human) genome assembly GRCh38, hg38, and/or Genome Reference Consortium Human Build 38. Those of skill in the art will further appreciate that DNA regions of hg38 can be referred to by a known system including identification of particular nucleotide positions or ranges thereof in accordance with assigned numbering.

Downstream: As used herein, the term “downstream” means that a first DNA region is closer, relative to a second DNA region, to the C-terminus of a nucleic acid that includes the first DNA region and the second DNA region.

Gene: As used herein, the term “gene” refers to a single DNA region, e.g., in a chromosome, that includes a coding sequence that encodes a product (e.g., an RNA product and/or a polypeptide product), together with all, some, or none of the DNA sequences that contribute to regulation of the expression of coding sequence. In some embodiments, e.g., as set forth herein, a gene includes one or more non-coding sequences. In some particular embodiments, e.g., as set forth herein, a gene includes exonic and intronic sequences. In some embodiments, e.g., as set forth herein, a gene includes one or more regulatory elements that, for example, can control or impact one or more aspects of gene expression (e.g., cell-type-specific expression, inducible expression, etc.). In some embodiments, e.g., as set forth herein, a gene includes a promoter. In some embodiments, e.g., as set forth herein, a gene includes one or both of a (i) DNA nucleotides extending a predetermined number of nucleotides upstream of the coding sequence and (ii) DNA nucleotides extending a predetermined number of nucleotides downstream of the coding sequence. In various embodiments, e.g., as set forth herein, the predetermined number of nucleotides can be 500 bp, 1 kb, 2 kb, 3 kb, 4 kb, 5 kb, 10 kb, 20 kb, 30 kb, 40 kb, 50 kb, 75 kb, or 100 kb.

Homology: As used herein, the term “homology” refers to the overall relatedness between polymeric molecules, e.g., between nucleic acid molecules (e.g., DNA molecules and/or RNA molecules) and/or between polypeptide molecules. Those of skill in the art will appreciate that homology can be defined, e.g., by a percent identity or by a percent homology (sequence similarity). In some embodiments, e.g., as set forth herein, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, e.g., as set forth herein, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar.

Hybridize: As used herein, “hybridize” refers to the association of a first nucleic acid with a second nucleic acid to form a double-stranded structure, which association occurs through complementary pairing of nucleotides. Those of skill in the art will recognize that complementary sequences, among others, can hybridize. In various embodiments, e.g., as set forth herein, hybridization can occur, for example, between nucleotide sequences having at least 70% complementarity, e.g., at least 75%, 80%, 85%, 90%, 91%, 92%, 93%, 94%, 95%, 96%, 97%, 98%, 99%, or 100% complementarity. Those of skill in the art will further appreciate that whether hybridization of a first nucleic acid and a second nucleic acid does or does not occur can dependence upon various reaction conditions. Conditions under which hybridization can occur are known in the art.

Hypomethylation: As used herein, the term “hypomethylation” refers to the state of a methylation locus having at least one fewer methylated nucleotides in a state of interest as compared to a reference state (e.g., at least one fewer methylated nucleotides in colorectal cancer than in a healthy control).

Hypermethylation: As used herein, the term “hypermethylation” refers to the state of a methylation locus having at least one more methylated nucleotide in a state of interest as compared to a reference state (e.g., at least one more methylated nucleotide in colorectal cancer than in a healthy control).

Identity, identical: As used herein, the terms “identity” and “identical” refers to the overall relatedness between polymeric molecules, e.g., between nucleic acid molecules (e.g., DNA molecules and/or RNA molecules) and/or between polypeptide molecules. Methods for the calculation of a percent identity as between two provided sequences are known in the art. Calculation of the percent identity of two nucleic acid or polypeptide sequences, for example, can be performed by aligning the two sequences (or the complement of one or both sequences) for optimal comparison purposes (e.g., gaps can be introduced in one or both of a first and a second sequences for optimal alignment and non-identical sequences can be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences and, optionally, taking into account the number of gaps and the length of each gap, which may need to be introduced for optimal alignment of the two sequences. The comparison of sequences and determination of percent identity between two sequences can be accomplished using a computational algorithm, such as BLAST (basic local alignment search tool).

“Improved,” “increased,” or “reduced”: As used herein, these terms, or grammatically comparable comparative terms, indicate values that are relative to a comparable reference measurement. For example, in some embodiments, e.g., as set forth herein, an assessed value achieved with an agent of interest may be “improved” relative to that obtained with a comparable reference agent or with no agent. Alternatively or additionally, in some embodiments, e.g., as set forth herein, an assessed value in a subject or system of interest may be “improved” relative to that obtained in the same subject or system under different conditions or at a different point in time (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, e.g., as set forth herein, comparative terms refer to statistically relevant differences (e.g., differences of a prevalence and/or magnitude sufficient to achieve statistical relevance). Those of skill in the art will be aware, or will readily be able to determine, in a given context, a degree and/or prevalence of difference that is required or sufficient to achieve such statistical significance.

Methylation: As used herein, the term “methylation” includes methylation at any of (i) C5 position of cytosine; (ii) N4 position of cytosine; and (iii) the N6 position of adenine. Methylation also includes (iv) other types of nucleotide methylation. A nucleotide that is methylated can be referred to as a “methylated nucleotide” or “methylated nucleotide base.” In certain embodiments, e.g., as set forth herein, methylation specifically refers to methylation of cytosine residues. In some instances, methylation specifically refers to methylation of cytosine residues present in CpG sites.

Methylation assay: As used herein, the term “methylation assay” refers to any technique that can be used to determine the methylation status of a methylation locus.

Methylation biomarker: As used herein, the term “methylation biomarker” refers to a biomarker that is or includes at least one methylation locus and/or the methylation status of at least one methylation locus, e.g., a hypermethylated locus. In particular, a methylation biomarker is a biomarker characterized by a change between a first state and a second state (e.g., between a cancerous state and a non-cancerous state) in methylation status of one or more nucleic acid loci.

Methylation locus: As used herein, the term “methylation locus” refers to a DNA region that includes at least one differentially methylated region. A methylation locus that includes a greater number or frequency of methylated sites under a selected condition of interest, such as a cancerous state, can be referred to as a hypermethylated locus. A methylation locus that includes a smaller number or frequency of methylated sites under a selected condition of interest, such as a cancerous state, can be referred to as a hypomethylated locus. In some instances, e.g., as set forth herein, a methylation locus has a length of at least 10, at least 15, at least 20, at least 30, at least 50, at least 75, at least 100, at least 121 base pairs. In some instances, e.g., as set forth herein, a methylation locus has a length of less than 5,000 bp, 4,000 bp, 3,000 bp, 2,200 bp, 2,101 bp, 2,000 bp, 1,000 bp, 950 bp, 900 bp, 850 bp, 800 bp, 750 bp, 700 bp, 650 bp, 600 bp, 550 bp, 500 bp, 450 bp, 400 bp, 350 bp, 300 bp, 250 bp, 200 bp, 150 bp, 100 bp, 50 bp, 40 bp, 30 bp, 20 bp, or 10 bp (e.g., where methylation status is determined using quantitative polymerase chain reaction (qPCR), e.g., methylation sensitive restriction enzyme quantitative polymerase chain reaction (MSRE-qPCR)).

Methylation site: As used herein, a methylation site refers to a nucleotide or nucleotide position that is methylated in at least one condition. In its methylated state, a methylation site can be referred to as a methylated site.

Methylation status: As used herein, “methylation status,” “methylation state,” or “methylation profile” refer to the number, frequency, or pattern of methylation at methylation sites within a methylation locus. Accordingly, a change in methylation status between a first state and a second state can be or include an increase in the number, frequency, or pattern of methylated sites, or can be or include a decrease in the number, frequency, or pattern of methylated sites. In various instances, a change in methylation status in a change in methylation value.

Nucleic acid: As used herein, in its broadest sense, the term “nucleic acid” refers to any compound and/or substance that is or can be incorporated into an oligonucleotide chain. In some embodiments e.g., as set forth herein, a nucleic acid is a compound and/or substance that is or can be incorporated into an oligonucleotide chain via a phosphodiester linkage. As will be clear from context, in some embodiments e.g., as set forth herein, the term nucleic acid refers to an individual nucleic acid residue (e.g., a nucleotide and/or nucleoside), and in some embodiments e.g., as set forth herein refers to a polynucleotide chain comprising a plurality of individual nucleic acid residues. A nucleic acid can be or include DNA, RNA, or any combination(s) thereof. A nucleic acid can include natural nucleic acid residues, nucleic acid analogs, and/or synthetic residues. In some embodiments e.g., as set forth herein, a nucleic acid includes natural nucleotides (e.g., adenosine, thymidine, guanosine, cytidine, uridine, deoxyadenosine, deoxythymidine, deoxy guanosine, and deoxycytidine). In some embodiments e.g., as set forth herein, a nucleic acid is or includes of one or more nucleotide analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0 (6)-methylguanine, 2-thiocytidine, methylated bases, intercalated bases, and combinations thereof).

In some embodiments e.g., as set forth herein, a nucleic acid has a nucleotide sequence that encodes a functional gene product such as an RNA or protein. In some embodiments e.g., as set forth herein, a nucleic acid includes one or more introns. In some embodiments e.g., as set forth herein, a nucleic acid includes one or more genes. In some embodiments e.g., as set forth herein, nucleic acids are prepared by one or more of isolation from a natural source, enzymatic synthesis by polymerization based on a complementary template (in vivo or in vitro), reproduction in a recombinant cell or system, and chemical synthesis.

In some embodiments e.g., as set forth herein, a nucleic acid analog differs from a nucleic acid in that it does not utilize a phosphodiester backbone. For example, in some embodiments e.g., as set forth herein, a nucleic acid can include one or more peptide nucleic acids, which are known in the art and have peptide bonds instead of phosphodiester bonds in the backbone. Alternatively or additionally, in some embodiments e.g., as set forth herein, a nucleic acid has one or more phosphorothioate and/or 5′-N-phosphoramidite linkages rather than phosphodiester bonds. In some embodiments e.g., as set forth herein, a nucleic acid comprises one or more modified sugars (e.g., 2′-fluororibose, ribose, 2′-deoxyribose, arabinose, and hexose) as compared with those in natural nucleic acids.

In some embodiments, e.g., as set forth herein, a nucleic acid is or includes at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 600, 700, 800, 900, 1000, 1500, 2000, 2500, 3000, 3500, 4000, 4500, 5000 or more residues. In some embodiments, e.g., as set forth herein, a nucleic acid is partly or wholly single stranded, or partly or wholly double stranded.

Nucleic acid detection assay: As used herein, the term “nucleic acid detection assay” refers to any method of determining the nucleotide composition of a nucleic acid of interest. Nucleic acid detection assays include but are not limited to, DNA sequencing methods (e.g., next generation sequencing methods), polymerase chain reaction-based methods, probe hybridization methods, ligase chain reaction, etc.

Nucleotide: As used herein, the term “nucleotide” refers to a structural component, or building block, of polynucleotides, e.g., of DNA and/or RNA polymers. A nucleotide includes of a base (e.g., adenine, thymine, uracil, guanine, or cytosine) and a molecule of sugar and at least one phosphate group. As used herein, a nucleotide can be a methylated nucleotide or an un-methylated nucleotide. Those of skill in the art will appreciate that nucleic acid terminology, such as, as examples, “locus” or “nucleotide” can refer to both a locus or nucleotide of a single nucleic acid molecule and/or to the cumulative population of loci or nucleotides within a plurality of nucleic acids (e.g., a plurality of nucleic acids in a sample and/or representative of a subject) that are representative of the locus or nucleotide (e.g., having the same identical nucleic acid sequence and/or nucleic acid sequence context, or having a substantially identical nucleic acid sequence and/or nucleic acid context).

Oligonucleotide primer: As used herein, the term oligonucleotide primer, or primer, refers to a nucleic acid molecule used, capable of being used, or for use in, generating amplicons from a template nucleic acid molecule. Under transcription-permissive conditions (e.g., in the presence of nucleotides and a DNA polymerase, and at a suitable temperature and pH), an oligonucleotide primer can provide a point of initiation of transcription from a template to which the oligonucleotide primer hybridizes. Typically, an oligonucleotide primer is a single-stranded nucleic acid between 5 and 200 nucleotides in length. Those of skill in the art will appreciate that optimal primer length for generating amplicons from a template nucleic acid molecule can vary with conditions including temperature parameters, primer composition, and transcription or amplification method. A pair of oligonucleotide primers, as used herein, refers to a set of two oligonucleotide primers that are respectively complementary to a first strand and a second strand of a template double-stranded nucleic acid molecule. First and second members of a pair of oligonucleotide primers may be referred to as a “forward” oligonucleotide primer and a “reverse” oligonucleotide primer, respectively, with respect to a template nucleic acid strand, in that the forward oligonucleotide primer is capable of hybridizing with a nucleic acid strand complementary to the template nucleic acid strand, the reverse oligonucleotide primer is capable of hybridizing with the template nucleic acid strand, and the position of the forward oligonucleotide primer with respect to the template nucleic acid strand is 5′ of the position of the reverse oligonucleotide primer sequence with respect to the template nucleic acid strand. It will be understood by those of skill in the art that the identification of a first and second oligonucleotide primer as forward and reverse oligonucleotide primers, respectively, is arbitrary inasmuch as these identifiers depend upon whether a given nucleic acid strand, or its complement is utilized as a template nucleic acid molecule.

Overlapping: The term “overlapping” is used herein in reference to two regions of DNA, each of which contains a sub-sequence that is substantially identical to a sub-sequence of the same length in the other region (e.g., the two regions of DNA have a common sub-sequence). “Substantially identical” means that the two identically-long sub-sequences differ by fewer than a given number of base pairs. In certain instances, e.g., as set forth herein, each sub-sequence has a length of at least 20 base pairs that differ by fewer than 4, 3, 2, or 1 base pairs from each other (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, each sub-sequence has a length of at least 24 base pairs that differ by fewer than 5, 4, 3, 2, or 1 base pairs (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, each sub-sequence has a length of at least 50 base pairs that differ by fewer than 10, 9, 8, 7, 6, 5, 4, 3, 2, or 1 base pairs (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, each sub-sequence has a length of at least 100 base pairs that differ by fewer than 20, 15, 10, 9, 8, 7, 6, 5, 4, 3, 2, or 1 base pairs (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, each sub-sequence has a length of at least 200 base pairs that differ by fewer than 40, 30, 20, 15, 10, 9, 8, 7, 6, 5, 4, 3, 2, or 1 base pairs (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, each sub-sequence has a length of at least 250 base pairs that differ by fewer than 50, 40, 30, 20, 15, 10, 9, 8, 7, 6, 5, 4, 3, 2, or 1 base pairs (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, each sub-sequence has a length of at least 300 base pairs that differ by fewer than 60, 50, 40, 30, 20, 15, 10, 9, 8, 7, 6, 5, 4, 3, 2, or 1 base pairs (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, each sub-sequence has a length of at least 500 base pairs that differ by fewer than 100, 60, 50, 40, 30, 20, 15, 10, 9, 8, 7, 6, 5, 4, 3, 2, or 1 base pairs (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, each sub-sequence has a length of at least 1000 base pairs that differ by fewer than 200, 100, 60, 50, 40, 30, 20, 15, 10, 9, 8, 7, 6, 5, 4, 3, 2, or 1 base pairs (e.g., the two sub-sequences having at least 80%, at least 85%, at least 90%, at least 95% similarity, at least 97% similarity, at least 98% similarity, at least 99% similarity, or at least 99.5% similarity). In certain instances, e.g., as set forth herein, the subsequence of a first region of the two regions of DNA may comprise the entirety of the second region of the two regions of DNA (or vice versa) (e.g., the common sub-sequence may contain the whole of either or both regions). In certain embodiments, where a methylation locus has a sequence that comprises at “least a portion of” a DMR sequence listed herein (e.g., at least 20%, at least 30%, at least 40%, at least 50%, at least 60%, at least 70%, at least 80%, or at least 90% of the DMR sequence), the overlapping portion of the methylation locus has at least 95% similarity, at least 98% similarity, or at least 99% similarity with the overlapping portion of the DMR sequence (e.g., if the overlapping portion is 100 bp, the portion of the methylation locus that overlaps with the portion of the DMR differs by no more than 1 bp, no more than 2 bp, or no more than 5 bp). In certain embodiments, where a methylation locus has a sequence that comprises “at least a portion of” a DMR sequence listed herein, this means the methylation locus has a subsequence in common with the DMR sequence that has a consecutive series of bases that covers at least 20%, at least 30%, at least 40%, at least 50%, at least 60%, at least 70%, at least 80%, or at least 90% of the DMR sequence, e.g., wherein the subsequence in common differs by no more than 1 bp, no more than 2 bp, or no more than 5 bp). In certain embodiments, where a methylation locus has a sequence that comprises “at least a portion of” a DMR sequence listed herein, this means the methylation locus contains at least a portion of (e.g., at least 20%, at least 30%, at least 40%, at least 50%, at least 60%, at least 70%, at least 80%, or at least 90% of) the CpG dinucleotides corresponding to the CpG dinucleotides within the DMR sequence.

Polyposis syndromes: The terms “polyposis” and “polyposis syndrome”, as used herein, refer to hereditary conditions that include, but are not limited to, familial adenomatous polyposis (FAP), hereditary nonpolyposis colorectal cancer (HNPCC)/Lynch syndrome, Gardner syndrome, Turcot syndrome, MUTYH polyposis, Peutz-Jeghers syndrome, Cowden disease, familial juvenile polyposis, and hyperplastic polyposis. In certain embodiments, polyposis includes serrated polyposis syndrome. Serrated polyposis is classified by a subject having 5 or more serrated polyps proximal to the sigmoid colon with two or more at least 10 mm in size, having a serrated polyp proximal to the sigmoid colon in the context of a family history of serrated polyposis, and/or having 20 or more serrated polyps throughout the colon.

Prevent or prevention: The terms “prevent” and “prevention,” as used herein in connection with the occurrence of a disease, disorder, or condition, refers to reducing the risk of developing the disease, disorder, or condition; delaying onset of the disease, disorder, or condition; delaying onset of one or more characteristics or symptoms of the disease, disorder, or condition; and/or to reducing the frequency and/or severity of one or more characteristics or symptoms of the disease, disorder, or condition. Prevention can refer to prevention in a particular subject or to a statistical impact on a population of subjects. Prevention can be considered complete when onset of a disease, disorder, or condition has been delayed for a predefined period of time.

Probe: As used herein, the terms “probe,” “capture probe,” or “bait” refer to a single- or double-stranded nucleic acid molecule that is capable of hybridizing with a complementary target and, in certain embodiments, includes a detectable moiety. In certain embodiments, e.g., as set forth herein, a probe is a restriction digest product or is a synthetically produced nucleic acid, e.g., a nucleic acid produced by recombination or amplification. In some instances, e.g., as set forth herein, a probe is a capture probe useful in detection, identification, and/or isolation of a target sequence, such as a gene sequence. In various instances, e.g., as set forth herein, a detectable moiety of probe can be, e.g., an enzyme (e.g., ELISA, as well as enzyme-based histochemical assays), fluorescent moiety, radioactive moiety, or moiety associated with a luminescence signal.

Prognosis: As used herein, the term “prognosis” refers to determining the qualitative of quantitative probability of at least one possible future outcome or event. As used herein, a prognosis can be a determination of the likely course of a disease, disorder, or condition such as cancer in a subject, a determination regarding the life expectancy of a subject, or a determination regarding response to therapy, e.g., to a particular therapy.

Prognostic information: As used herein, the term “prognostic information” refers to information useful in providing a prognosis. Prognostic information can include, without limitation, biomarker status information.

Promoter: As used herein, a “promoter” can refer to a DNA regulatory region that directly or indirectly (e.g., through promoter-bound proteins or substances) associates with an RNA polymerase and participates in initiation of transcription of a coding sequence.

Reference: As used herein describes a standard or control relative to which a comparison is performed. For example, in some embodiments, e.g., as set forth herein, an agent, subject, animal, individual, population, sample, sequence, or value of interest is compared with a reference or control agent, subject, animal, individual, population, sample, sequence, or value. In some embodiments, e.g., as set forth herein, a reference or characteristic thereof is tested and/or determined substantially simultaneously with the testing or determination of the characteristic in a sample of interest. In some embodiments, e.g., as set forth herein, a reference is a historical reference, optionally embodied in a tangible medium. Typically, as would be understood by those of skill in the art, a reference is determined or characterized under comparable conditions or circumstances to those under assessment, e.g., with regard to a sample. Those skilled in the art will appreciate when sufficient similarities are present to justify reliance on and/or comparison to a particular possible reference or control.

Risk: As used herein with respect to a disease, disorder, or condition, the term “risk” refers to the qualitative of quantitative probability (whether expressed as a percentage or otherwise) that a particular individual will develop the disease, disorder, or condition. In some embodiments, e.g., as set forth herein, risk is expressed as a percentage. In some embodiments, e.g., as set forth herein, a risk is a qualitative of quantitative probability that is equal to or greater than 0, 1, 2, 3, 4, 5, 6, 7, 8, 9, 10, 20, 30, 40, 50, 60, 70, 80, 90, or 100%. In some embodiments, e.g., as set forth herein, risk is expressed as a qualitative or quantitative level of risk relative to a reference risk or level or the risk of the same outcome attributed to a reference. In some embodiments, e.g., as set forth herein, relative risk is increased or decreased in comparison to the reference sample by a factor of 1.1, 1.2, 1.3, 1.4, 1.5, 1.6, 1.7, 1.8, 1.9, 2, 3, 4, 5, 6, 7, 8, 9, 10, or more.

Sample: As used herein, the term “sample” typically refers to an aliquot of material obtained or derived from a source of interest. In some embodiments, e.g., as set forth herein, a source of interest is a biological or environmental source. In some embodiments, e.g., as set forth herein, a sample is a “primary sample” obtained directly from a source of interest. In some embodiments, e.g., as set forth herein, as will be clear from context, the term “sample” refers to a preparation that is obtained by processing of a primary sample (e.g., by removing one or more components of and/or by adding one or more agents to a primary sample). Such a “processed sample” can include, for example cells, nucleic acids, or proteins extracted from a sample or obtained by subjecting a primary sample to techniques such as amplification or reverse transcription of nucleic acids, isolation and/or purification of certain components, etc.

In certain instances, e.g., as set forth herein, a processed sample can be a DNA sample that has been amplified (e.g., pre-amplified). Thus, in various instances, e.g., as set forth herein, an identified sample can refer to a primary form of the sample or to a processed form of the sample. In some instances, e.g., as set forth herein, a sample that is enzyme-digested DNA can refer to primary enzyme-digested DNA (the immediate product of enzyme digestion) or a further processed sample such as enzyme-digested DNA that has been subject to an amplification step (e.g., an intermediate amplification step, e.g., pre-amplification) and/or to a filtering step, purification step, or step that modifies the sample to facilitate a further step, e.g., in a process of determining methylation status (e.g., methylation status of a primary sample of DNA and/or of DNA as it existed in its original source context).

Screening: As used herein, the term “screening” refers to any method, technique, process, or undertaking intended to generate diagnostic information and/or prognostic information. Accordingly, those of skill in the art will appreciate that the term screening encompasses method, technique, process, or undertaking that determines whether an individual has, is likely to have or develop, or is at risk of having or developing a disease, disorder, or condition, e.g., colorectal cancer, advanced adenoma.

Specificity: As used herein, the “specificity” of a biomarker refers to the percentage of samples that are characterized by absence of the event or state of interest for which measurement of the biomarker accurately indicates absence of the event or state of interest (true negative rate). In various embodiments, e.g., as set forth herein, characterization of the negative samples is independent of the biomarker, and can be achieved by any relevant measure, e.g., any relevant measure known to those of skill in the art. Thus, specificity reflects the probability that the biomarker would detect the absence of the event or state of interest when measured in a sample not characterized as having that event or state of interest. In particular embodiments in which the event or state of interest is colorectal cancer, e.g., as set forth herein, specificity refers to the probability that a biomarker would detect the absence of colorectal cancer in a subject lacking colorectal cancer. Lack of colorectal cancer can be determined, e.g., by histology.

Sensitivity: As used herein, the “sensitivity” of a biomarker refers to the percentage of samples that are characterized by the presence of the event or state of interest for which measurement of the biomarker accurately indicates presence of the event or state of interest (true positive rate). In various embodiments, e.g., as set forth herein, characterization of the positive samples is independent of the biomarker, and can be achieved by any relevant measure, e.g., any relevant measure known to those of skill in the art. Thus, sensitivity reflects the probability that a biomarker would detect the presence of the event or state of interest when measured in a sample characterized by presence of that event or state of interest. In particular embodiments in which the event or state of interest is colorectal cancer, e.g., as set forth herein, sensitivity refers to the probability that a biomarker would detect the presence of colorectal cancer in a subject that has colorectal cancer. Presence of colorectal cancer can be determined, e.g., by histology.

Stage of cancer: As used herein, the term “stage of cancer” refers to a qualitative or quantitative assessment of the level of advancement of a cancer. In some embodiments, e.g., as set forth herein, criteria used to determine the stage of a cancer can include, but are not limited to, one or more of where the cancer is located in a body, tumor size, whether the cancer has spread to lymph nodes, whether the cancer has spread to one or more different parts of the body, etc. In some embodiments, e.g., as set forth herein, cancer can be staged using the so-called TNM System, according to which T refers to the size and extent of the main tumor, usually called the primary tumor; N refers to the number of nearby lymph nodes that have cancer; and M refers to whether the cancer has metastasized. In some embodiments, e.g., as set forth herein, a cancer can be referred to as Stage 0 (abnormal cells are present but have not spread to nearby tissue, also called carcinoma in situ, or CIS; CIS is not cancer, but it can become cancer), Stage I-III (cancer is present; the higher the number, the larger the tumor and the more it has spread into nearby tissues), or Stage IV (the cancer has spread to distant parts of the body). In some embodiments, e.g., as set forth herein, a cancer can be assigned to a stage selected from the group consisting of: in situ (abnormal cells are present but have not spread to nearby tissue); localized (cancer is limited to the place where it started, with no sign that it has spread); regional (cancer has spread to nearby lymph nodes, tissues, or organs): distant (cancer has spread to distant parts of the body); and unknown (there is not enough information to identify cancer stage).

Susceptible to: An individual who is “susceptible to” a disease, disorder, or condition is at risk for developing the disease, disorder, or condition. In some embodiments, e.g., as set forth herein, an individual who is susceptible to a disease, disorder, or condition does not display any symptoms of the disease, disorder, or condition. In some embodiments, e.g., as set forth herein, an individual who is susceptible to a disease, disorder, or condition has not been diagnosed with the disease, disorder, and/or condition. In some embodiments, e.g., as set forth herein, an individual who is susceptible to a disease, disorder, or condition is an individual who has been exposed to conditions associated with, or presents a biomarker status (e.g., a methylation status) associated with, development of the disease, disorder, or condition. In some embodiments, e.g., as set forth herein, a risk of developing a disease, disorder, and/or condition is a population-based risk (e.g., family members of individuals suffering from the disease, disorder, or condition).

Subject: As used herein, the term “subject” refers to an organism, typically a mammal (e.g., a human). In some embodiments, e.g., as set forth herein, a subject is suffering from a disease, disorder or condition. In some embodiments, e.g., as set forth herein, a subject is susceptible to a disease, disorder, or condition. In some embodiments, e.g., as set forth herein, a subject displays one or more symptoms or characteristics of a disease, disorder or condition. In some embodiments, e.g., as set forth herein, a subject is not suffering from a disease, disorder or condition. In some embodiments, e.g., as set forth herein, a subject does not display any symptom or characteristic of a disease, disorder, or condition. In some embodiments, e.g., as set forth herein, a subject is someone with one or more features characteristic of susceptibility to or risk of a disease, disorder, or condition. In some embodiments, e.g., as set forth herein, a subject is a patient. In some embodiments, e.g., as set forth herein, a subject is an individual to whom diagnosis has been performed and/or to whom therapy has been administered. In some instances, e.g., as set forth herein, a human subject can be interchangeably referred to as an “individual.”

Treatment: As used herein, the term “treatment” (also “treat” or “treating”) refers to administration of a therapy that partially or completely alleviates, ameliorates, relieves, inhibits, delays onset of, reduces severity of, and/or reduces incidence of one or more symptoms, features, and/or causes of a particular disease, disorder, or condition, or is administered for the purpose of achieving any such result. In some embodiments, e.g., as set forth herein, such treatment can be of a subject who does not exhibit signs of the relevant disease, disorder, or condition and/or of a subject who exhibits only early signs of the disease, disorder, or condition. Alternatively or additionally, such treatment can be of a subject who exhibits one or more established signs of the relevant disease, disorder and/or condition. In some embodiments, e.g., as set forth herein, treatment can be of a subject who has been diagnosed as suffering from the relevant disease, disorder, and/or condition. In some embodiments, e.g., as set forth herein, treatment can be of a subject known to have one or more susceptibility factors that are statistically correlated with increased risk of development of the relevant disease, disorder, or condition. In various examples, treatment is of a cancer.

Upstream: As used herein, the term “upstream” means a first DNA region is closer, relative to a second DNA region, to the N-terminus of a nucleic acid that includes the first DNA region and the second DNA region.

Unit dose: As used herein, the term “unit dose” refers to an amount administered as a single dose and/or in a physically discrete unit of a pharmaceutical composition. In many embodiments, e.g., as set forth herein, a unit dose contains a predetermined quantity of an active agent. In some embodiments, e.g., as set forth herein, a unit dose contains an entire single dose of the agent. In some embodiments, e.g., as set forth herein, more than one unit dose is administered to achieve a total single dose. In some embodiments, e.g., as set forth herein, administration of multiple unit doses is required, or expected to be required, in order to achieve an intended effect. A unit dose can be, for example, a volume of liquid (e.g., an acceptable carrier) containing a predetermined quantity of one or more therapeutic moieties, a predetermined amount of one or more therapeutic moieties in solid form, a sustained release formulation or drug delivery device containing a predetermined amount of one or more therapeutic moieties, etc. It will be appreciated that a unit dose can be present in a formulation that includes any of a variety of components in addition to the therapeutic agent(s). For example, acceptable carriers (e.g., pharmaceutically acceptable carriers), diluents, stabilizers, buffers, preservatives, etc., can be included. It will be appreciated by those skilled in the art, in many embodiments, e.g., as set forth herein, a total appropriate daily dosage of a particular therapeutic agent can comprise a portion, or a plurality, of unit doses, and can be decided, for example, by a medical practitioner within the scope of sound medical judgment. In some embodiments, e.g., as set forth herein, the specific effective dose level for any particular subject or organism can depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

Unmethylated: As used herein, the terms “unmethylated” and “non-methylated” are used interchangeably and mean that an identified DNA region includes no methylated nucleotides.

### BRIEF DESCRIPTION OF THE DRAWINGS

The foregoing and other objects, aspects, features, and advantages of the present disclosure will become more apparent and better understood by referring to the following description taken in conjunction with the accompanying drawings, in which:

FIG. 1 shows overall data relating to the sensitivity and specificity of the DMR chr2: 29920045-29921364 (SEQ ID NO. 1), according to an illustrative embodiment.

FIG. 2 shows overall data relating to the sensitivity and specificity of the DMR chr1: 107963936-107966036 (SEQ ID NO. 2), according to an illustrative embodiment.

FIG. 3 shows overall data relating to the sensitivity and specificity of the DMR chr2: 73292434-73292554 (SEQ ID NO. 3), according to an illustrative embodiment.

FIG. 4 shows overall data relating to the sensitivity and specificity of the DMR chr5: 178590240-178590360 (SEQ ID NO. 4), according to an illustrative embodiment.

FIG. 5 shows overall data relating to the sensitivity and specificity of the DMR chr5: 173234839-173234959 (SEQ ID NO. 5), according to an illustrative embodiment.

FIG. 6 shows overall data relating to the sensitivity and specificity of the DMR chr20: 62476374-62476494 (SEQ ID NO. 6), according to an illustrative embodiment.

FIG. 7 shows overall data relating to the sensitivity and specificity of the DMR chr8: 72251358-72251490 (SEQ ID NO. 7), according to an illustrative embodiment.

FIG. 8 shows overall data relating to the sensitivity and specificity of the DMR chr2: 236237696-236237816 (SEQ ID NO. 8), according to an illustrative embodiment.

FIG. 9 shows overall data relating to the sensitivity and specificity of the DMR chr6: 105981552-105981672 (SEQ ID NO. 9), according to an illustrative embodiment.

FIG. 10 shows overall data relating to the sensitivity and specificity of the DMR chr20: 63177004-63178804 (SEQ ID NO. 10), according to an illustrative embodiment.

FIG. 11 shows overall data relating to the sensitivity and specificity of the DMR chr2: 100321258-100322771 (SEQ ID NO. 11), according to an illustrative embodiment.

FIG. 12 shows overall data relating to the sensitivity and specificity of the DMR chr4: 143699944-143701144 (SEQ ID NO. 12), according to an illustrative embodiment.

FIG. 13 shows out-of-bag (OOB) scores of combinations of two markers, according to an illustrative embodiment.

FIG. 14 shows OOB scores of combinations of three markers, according to an illustrative embodiment.

FIG. 15 shows OOB scores of combinations of four markers and a five marker combination, according to an illustrative embodiment.

FIG. 16 shows OOB scores of combinations of two and three markers, according to an illustrative embodiment.

FIG. 17 is a block diagram of an exemplary process (1700) for detecting methylation status used in certain embodiments.

FIG. 18 is a block diagram of an exemplary cloud computing environment, used in certain embodiments.

FIG. 19 is a block diagram of an example computing device and an example mobile computing device used in certain embodiments.

### DETAILED DESCRIPTION

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Documents are incorporated herein by reference as noted. Where there is any discrepancy in the meaning of a particular term, the meaning provided in the Definition section above is controlling.

Headers are provided for the convenience of the reader—the presence and/or placement of a header is not intended to limit the scope of the subject matter described herein.

### Detection of Colorectal Cancer and Advanced Adenoma

In various embodiments, a methylation biomarker of the present disclosure used for detection of colorectal cancer and/or advanced adenoma is selected from a methylation locus that is or includes at least a portion of a DMR listed in Table 1 below.

TABLE 1
DMR and Gene List.

| Chromosome | | | | SEQ |
|---|---|---|---|---|
| No. | Start | End | Gene | ID NO. |
| 2 | 29920045 | 29921364 | ALK | 1 |
| 1 | 107963936 | 107966036 | VAV3 | 2 |
| 2 | 73292434 | 73292554 | EGR4 | 3 |
| 5 | 178590240 | 178590360 | COL23A1 | 4 |
| 5 | 173234839 | 173234959 | NKX2-5 | 5 |
| 20 | 62476374 | 62476494 | GATA5 | 6 |
| 8 | 72251358 | 72251490 | AC022905.1 | 7 |
| 2 | 236237696 | 236237816 | ASB18 | 8 |
| 6 | 105981552 | 105981672 | RN7SKP211 | 9 |
| 20 | 63177004 | 63178804 | MIR124-3 | 10 |
| 2 | 100321258 | 100322771 | LONRF2 | 11 |
| 4 | 143699944 | 143701144 | AC139713.2 | 12 |
| 5 | 132073832 | 132073952 | — | 13 |

Table 1 lists 13 DMRs which have been found to be useful in detection of colorectal cancer and/or advanced adenoma. The location of the DMR is found using the chromosome number (Chromosome No.), start, and end positions of the DMR. The locations correspond to locations found in the GRCh38 reference genome (or a version thereof). The start and end positions of the DMR assumes a 1-based reference where the start and end positions are inclusive. Each DMR in Table 1 corresponds to a gene with which the DMR overlaps (“Gene”). For the avoidance of any doubt, any methylation biomarker or gene provided in Table 1 can be, or be included in, among other things, a colorectal cancer marker and/or an advanced adenoma marker.

In some particular embodiments, combinations of DMRs have been found to be useful in the detection of colorectal cancer and/or advanced adenoma, for example, combinations of DMRs listed in Tables 2-10. [As used in this specification, “useful in the detection of colorectal cancer and/or advanced adenoma” means that in certain embodiments, any one or more of (i), (ii), and (iii) may apply: (i) the noted marker(s) are useful in the detection of colorectal cancer; (ii) in certain embodiments, the marker(s) are useful in the detection of advanced adenoma; and/or (iii) in certain embodiments, the marker(s) are useful in the undifferentiated detection of colorectal cancer or advanced adenoma.] Tables 2-10 list subsets of DMRs from Table 1. Genes, portions of DMRs, and combinations thereof specifically identified in Tables 2-10 are useful for identifying colorectal cancer and/or advanced adenoma. In certain embodiments, a methylation biomarker of the present disclosure used for detection of colorectal cancer and/or advanced adenoma is selected from a methylation locus that is or includes at least a portion of a DMR listed in Tables 2-10 below. In certain embodiments, a methylation locus that is or includes at least a portion of a DMR listed in Tables 2-10 is particularly useful as a colorectal cancer and/or advanced adenoma marker. In certain embodiments, a methylation locus that is or includes at least a portion of a gene listed in Tables 2-10 is particularly useful as a colorectal cancer and/or advanced adenoma marker. In some embodiments, Table 2 is particularly useful as a colorectal cancer marker. In some embodiments, Table 9 is particularly useful as a colorectal cancer marker. In some embodiments, Table 10 is particularly useful as a colorectal cancer marker.

TABLE 2
1 DMR only.

| Chromosome | | | | SEQ |
|---|---|---|---|---|
| No. | Start | End | Gene | ID NO. |
| 4 | 143699944 | 143701144 | AC139713.2 | 12 |

TABLE 3
Combination of 2 DMRs.

| Chromosome | | | | SEQ |
|---|---|---|---|---|
| No. | Start | End | Gene | ID NO. |
| 2 | 29920045 | 29921364 | ALK | 1 |
| 2 | 100321258 | 100322771 | LONRF2 | 11 |

TABLE 4
Combination of 2 DMRs.

| Chromosome | | | | SEQ |
|---|---|---|---|---|
| No. | Start | End | Gene | ID NO. |
| 1 | 107963936 | 107966036 | VAV3 | 2 |
| 20 | 63177004 | 63178804 | MIR124-3 | 10 |

TABLE 5
Combination of 2 DMRs.

| Chromosome | | | | SEQ |
|---|---|---|---|---|
| No. | Start | End | Gene | ID NO. |
| 1 | 107963936 | 107966036 | VAV3 | 2 |
| 4 | 143699944 | 143701144 | AC139713.2 | 12 |

TABLE 6
Combination of 4 DMRs.

| Chromosome | | | | SEQ |
|---|---|---|---|---|
| No. | Start | End | Gene | ID NO. |
| 1 | 107963936 | 107966036 | VAV3 | 2 |
| 20 | 62476374 | 62476494 | GATA5 | 6 |
| 20 | 63177004 | 63178804 | MIR124-3 | 10 |
| 2 | 100321258 | 100322771 | LONRF2 | 11 |

TABLE 7
Combination of 2 DMRs.

| Chromosome | | | | SEQ |
|---|---|---|---|---|
| No. | Start | End | Gene | ID NO. |
| 20 | 63177004 | 63178804 | MIR124-3 | 10 |
| 2 | 100321258 | 100322771 | LONRF2 | 11 |

TABLE 8
Combination of 7 DMRs.

| Chromosome | | | | SEQ |
|---|---|---|---|---|
| No. | Start | End | Gene | ID NO. |
| 1 | 107963936 | 107966036 | VAV3 | 2 |
| 20 | 62476374 | 62476494 | GATA5 | 6 |
| 8 | 72251358 | 72251490 | AC022905.1 | 7 |
| 20 | 63177004 | 63178804 | MIR124-3 | 10 |
| 2 | 100321258 | 100322771 | LONRF2 | 11 |
| 4 | 143699944 | 143701144 | AC139713.2 | 12 |
| 5 | 132073832 | 132073952 | — | 13 |

TABLE 9
Combination of 3 DMRs.

| Chromosome | | | | SEQ |
|---|---|---|---|---|
| No. | Start | End | Gene | ID NO. |
| 1 | 107963936 | 107966036 | VAV3 | 2 |
| 2 | 100321258 | 100322771 | LONRF2 | 11 |
| 20 | 63177004 | 63178804 | MIR124-3 | 20 |

TABLE 10
Combination of 4 DMRs.

| Chromosome | | | | SEQ |
|---|---|---|---|---|
| No. | Start | End | Gene | ID NO. |
| 1 | 107963936 | 107966036 | VAV3 | 2 |
| 2 | 100321258 | 100322771 | LONRF2 | 11 |
| 20 | 62476374 | 62476494 | GATA5 | 6 |
| 20 | 63177004 | 63178804 | MIR124-3 | 20 |

In some embodiments, said methylation biomarker can be or include a single methylation locus. In some embodiments, a methylation biomarker can be or include two or more methylation loci. In some embodiments, a methylation biomarker can be or include a single differentially methylated region (DMR) (e.g., (i) a DMR selected from those listed in Table 1, (ii) a DMR that encompasses a DMR selected from those listed in Table 1, (iii) a DMR that overlaps with one or more DMRs selected from those listed in Table 1, or (iv) a DMR that is a portion of a DMR selected from those listed in Table 1). In some embodiments, a methylation locus can be or include two or more DMRs (e.g., two, three, four, five, six, seven, eight, nine, ten, eleven, twelve, or thirteen DMRs selected from those listed in Table 1, or two, three, four, five, six, seven, eight, nine, ten, eleven, twelve, thirteen or more DMRs, each of which overlap with and/or encompass a DMR selected from those listed in Table 1). In some embodiments, a methylation biomarker can be or include a single methylation site (e.g., a single CpG site, a methylated cytosine residue). In other embodiments, a methylation biomarker can be or include two or more methylation sites. In some embodiments, a methylation locus can include two or more DMRs and further include DNA regions adjacent to one or more of the included DMRs.

In some instances, a methylation locus is or includes a gene, such as a gene provided in Table 1. In some instances, a methylation locus may be or include portions of a DMR provided in Table 1 which are not currently associated with any known gene.

Those of skill in the art will appreciate that a methylation locus identified as a methylation biomarker need not necessarily be assayed in a single experiment, reaction, or amplicon. A single methylation locus identified as a colorectal cancer methylation biomarker can be assayed, e.g., in a method including separate amplification (or providing oligonucleotide primers and conditions sufficient for amplification of) of one or more distinct or overlapping DNA regions within a methylation locus, e.g., one or more distinct or overlapping DMRs. Those of skill in the art will further appreciate that a methylation locus identified as a methylation biomarker need not be analyzed for methylation status of each nucleotide, nor each CpG, present within the methylation locus. Rather, a methylation locus that is a methylation biomarker may be analyzed, e.g., by analysis of a single DNA region within the methylation locus, e.g., by analysis of a single DMR within the methylation locus.

DMRs of the present disclosure can be a methylation locus or include a portion of a methylation locus. In some instances, a DMR is a DNA region with a methylation locus that is, e.g., 1 to 5,000 bp in length. In various embodiments, a DMR is a DNA region with a methylation locus that is equal to or less than 5,000 bp, 4,000 bp, 3,000 bp, 2,200 bp, 2,101 bp, 2,000 bp, 1,000 bp, 950 bp, 900 bp, 850 bp, 800 bp, 750 bp, 700 bp, 650 bp, 600 bp, 550 bp, 500 bp, 450 bp, 400 bp, 350 bp, 300 bp, 250 bp, 200 bp, 150 bp, 100 bp, 50 bp, 40 bp, 30 bp, 20 bp, or 10 bp in length, covering at least 1 methylated CpG. In some embodiments, a DMR covers at least 1, at least 2, at least 3, at least 4, at least 5, at least 6, at least 7, at least 8, or at least 9 CpGs. In some embodiments, a DMR is greater than 20 bp, 50 bp, 100 bp, 121 bp, 200 bp, or greater in length.

Methylation biomarkers, including without limitation methylation loci and DMRs provided herein, can include at least one methylation site that is a colorectal cancer biomarker.

For clarity, those of skill in the art will appreciate that the term “methylation biomarker” is used broadly, such that a methylation biomarker can include one or more methylation loci within a single DMR, and each methylation locus is also itself a methylation biomarker. Moreover, a single DMR can contain multiple methylation biomarkers. A methylation biomarker can be a subset of a single DMR, but a single methylation loci cannot span across multiple DMRs. Accordingly, status as a methylation biomarker does not turn on the contiguousness of nucleic acids included in a biomarker, but rather on the existence of a change in methylation status for included DNA region(s) between a first state and a second state, such as between colorectal cancer and controls, advanced adenoma and controls, or both colorectal cancer and advanced adenoma and controls. As provided herein, a methylation locus can be any of one or more methylation loci each of which methylation loci is, includes, or is a portion of a gene (or specific DMR) identified in Table 1. In some embodiments, a colorectal cancer and/or advanced adenoma methylation biomarker includes a single methylation locus that is, includes, or is a portion of a gene identified in Table 1.

In some embodiments, a methylation biomarker includes two or more methylation loci, each of which is, includes, or is a portion of a gene identified in Table 1. In some embodiments, a colorectal cancer and/or advanced adenoma methylation biomarker includes a plurality of methylation loci, each of which is, includes, or is a portion of a gene identified in Table 1.

In various embodiments, a methylation biomarker can be or include one or more individual nucleotides (e.g., a single individual cytosine residue in the context of a CpG) or a plurality of individual cytosine residues (e.g., of a plurality of CpGs) present within one or more methylation loci (e.g., one or more DMRs) provided herein. Thus, in certain embodiments a methylation biomarker is or includes methylation status of a plurality of individual methylation sites.

In various embodiments, a methylation biomarker is, includes, or is characterized by change in methylation status that is a change in the methylation of one or more methylation sites within one or more methylation loci (e.g., one or more DMRs). In various embodiments, a methylation biomarker is or includes a change in methylation status that is a change in the number of methylated sites within one or more methylation loci (e.g., one or more DMRs) (e.g., one or more CpG sites). In various embodiments, a methylation biomarker is or includes a change in methylation status that is a change in the frequency of methylation sites within one or more methylation loci (e.g., one or more DMRs). In various embodiments, a methylation biomarker is or includes a change in methylation status that is a change in the pattern of methylation sites within one or more methylation loci (e.g., one or more DMRs).

In various embodiments, methylation status of one or more methylation loci (e.g., one or more DMRs) is expressed as a fraction or percentage of the one or more methylation loci (e.g., the one or more DMRs) present in a sample that are methylated, e.g., as a fraction of the number of individual DNA strands of DNA in a sample that are methylated at one or more particular methylation loci (e.g., one or more particular DMRs). Those of skill in the art will appreciate that, in some instances, the fraction or percentage of methylation can be calculated from the ratio of methylated DMRs to unmethylated DMRs for one or more analyzed DMRs, e.g., within a sample.

In various embodiments, methylation status of one or more methylation loci (e.g., one or more DMRs) is compared to a reference methylation status value and/or to methylation status of the one or more methylation loci (e.g., one or more DMRs) in a reference sample or a group of reference samples. For example, in certain embodiments, the group of reference samples is a plurality of samples obtained from individuals where said samples are known to represent a particular state (e.g., a “normal” non-cancer state, or a cancer state). In certain instances, a reference is a non-contemporaneous sample from the same source, e.g., a prior sample from the same source, e.g., from the same subject. In certain instances, a reference for the methylation status of one or more methylation loci (e.g., one or more DMRs) is the methylation status of the one or more methylation loci (e.g., one or more DMRs) in a sample (e.g., a sample from a subject), or a plurality of samples, known to represent a particular state (e.g., a cancer state or a non-cancer state). Thus, a reference can be or include one or more predetermined thresholds, which thresholds can be quantitative (e.g., a methylation value) or qualitative. Those of skill in the art will appreciate that a reference measurement is typically produced by measurement using a methodology identical to, similar to, or comparable to that by which the non-reference measurement was taken.

In various embodiments, methylation status of one or more methylation loci (e.g., one or more DMRs) is compared to a reference methylation status value and/or to methylation status of the one or more methylation loci (e.g., one or more DMRs) in a reference sample. In certain instances, a reference is a non-contemporaneous sample from the same source, e.g., a prior sample from the same source, e.g., from the same subject. In certain instances, a reference for the methylation status of one or more methylation loci (e.g., one or more DMRs) is the methylation status of the one or more methylation loci (e.g., one or more DMRs) in a sample (e.g., a sample from a subject), or a plurality of samples, known to represent a particular state (e.g., a cancer state or a non-cancer state). Thus, a reference can be or include one or more predetermined thresholds, which thresholds can be quantitative (e.g., a methylation value) or qualitative. Those of skill in the art will appreciate that a reference measurement is typically produced by measurement using a methodology identical to, similar to, or comparable to that by which the non-reference measurement was taken.

In various embodiments, a methylation status of a methylation loci may be based on methylation of one or more reads (e.g., obtained using a NGS technique) mapped to the methylation loci. For example, when analyzing sequencing data obtained from a sequencing technique, e.g., a NGS sequencing technique, e.g., a targeted NGS sequencing technique, sequencing data may include an inferred or probabilistic sequence of base pairs of a DNA fragment. The inferred or probabilistic sequence of base pairs of the DNA fragment is known as a read. The read may be mapped to a methylation loci (e.g., a DMR, a mutation marker) reference sequence, for example, in a genome (e.g., a reference genome, e.g., a reference bisulfite converted genome). Based on a comparison of the read sequence to a reference sequence, individual CpGs or cytosine residues may be identified as being hypermethylated or hypomethylated as compared to a reference state. In certain embodiments, a read-wise methylation value (e.g., a read-wise methylation score) is determined for a read, based pre-determined minimal thresholds that takes into account a number of methylation sites (e.g., CpGs) and a percentage of methylation. For example, in a portion of a sequence read which overlaps a methylation marker (e.g., a DMR), at least 50% (e.g., at least 60%, at least 70%, at least 75%, at least 80%, at least 90%, or all) of CpGs in the overlapping portion being methylated are indicative that a sequence read is hypermethylated. In some embodiments, an overlapping portion of a sequence read and a methylation marker comprises at least 2 (e.g., at least 3, at least 4, at least 5, at least 6, at least 7, at least 8 or more) CpGs. In some embodiments, an overlapping portion of a sequence read and a methylation marker comprises 27 or fewer (e.g., 26 or fewer, 25 or fewer, 20 or fewer, 17 or fewer) CpGs.

### Advanced Adenomas

In certain embodiments, methods and compositions presented herein are useful for screening for advanced adenomas. Advanced adenomas include, without limitation: neoplastic adenomatous growth in colon and/or in rectum, adenomas located in the proximal part of the colon, adenomas located in the distal part of the colon and/or rectum, adenomas of low grade dysplasia, adenomas of high grade dysplasia, neoplastic growth(s) of colorectum tissue that shows signs of high grade dysplasia of any size, neoplastic growth(s) of colorectum tissue having a size greater than or equal to 10 mm of any histology and/or dysplasia grade, neoplastic growth(s) of colorectum tissue with villious histological type of any type of dysplasia and any size, and colorectum tissue having a serrated histological type with any dysplasia grade and/or size.

### Colorectal Cancers

In certain embodiments, methods and compositions of the present disclosure are useful for screening for colorectal cancer. Colorectal cancers include, without limitation, colon cancer, rectal cancer, and combinations thereof. Colorectal cancers include metastatic colorectal cancers and non-metastatic colorectal cancers. Colorectal cancers include cancer located in the proximal part of the colon cancer and cancer located in the distal part of the colon.

Colorectal cancers include colorectal cancers at any of the various possible stages known in the art, including, e.g., Stage 0, Stage I, Stage II, Stage III, and Stage IV colorectal cancers (e.g., stages 0, I, IIA, IIB, IIC, IIIA, IIIB, IIIC, IVA, IVB, and IVC). Colorectal cancers include all stages of the Tumor/Node/Metastasis (TNM) staging system. With respect to colorectal cancer, T can refer to whether the tumor grown into the wall of the colon or rectum, and if so by how many layers; N can refer to whether the tumor has spread to lymph nodes, and if so how many lymph nodes and where they are located; and M can refer to whether the cancer has spread to other parts of the body, and if so which parts and to what extent. Particular stages of T, N, and M are known in the art. T stages can include TX, T0, Tis, T1, T2, T3, T4a, and T4b; N stages can include NX, N0, N1a, N1b, N1c, N2a, and N2b; M stages can include M0, M1a, and M1b. Moreover, grades of colorectal cancer can include GX, G1, G2, G3, and G4. Various means of staging cancer, and colorectal cancer in particular, are well known in the art summarized, e.g., on the world wide web at cancer.net/cancer-types/colorectal-cancer/stages.

In certain instances, the present disclosure includes screening of early stage colorectal cancer. Early stage colorectal cancers can include, e.g., colorectal cancers localized within a subject, e.g., in that they have not yet spread to lymph nodes of the subject, e.g., lymph nodes near to the cancer (stage N0), and have not spread to distant sites (stage M0). Early stage cancers include colorectal cancers corresponding to, e.g., Stages 0 to II (inclusive of stages IIA, IIB, IIC).

Thus, colorectal cancers of the present disclosure include, among other things, pre-malignant colorectal cancer and malignant colorectal cancer. Methods and compositions of the present disclosure are useful for screening of colorectal cancer in all of its forms and stages, including without limitation those named herein or otherwise known in the art, as well as all subsets thereof. Accordingly, the person of skill in art will appreciate that all references to colorectal cancer provided here include, without limitation, colorectal cancer in all of its forms and stages, including without limitation those named herein or otherwise known in the art, as well as all subsets thereof.

### Subjects and Samples

A sample analyzed using methods and compositions provided herein can be any biological sample and/or any sample including nucleic acids. In various particular embodiments, a sample analyzed using methods and compositions provided herein can be a sample from a mammal. In various particular embodiments, a sample analyzed using methods and compositions provided herein can be a sample from a human subject. In various particular embodiments, a sample analyzed using methods and compositions provided herein can be a sample form a mouse, rat, pig, horse, chicken, or cow.

In various instances, a human subject is a subject diagnosed or seeking diagnosis as having, diagnosed as or seeking diagnosis as at risk of having, and/or diagnosed as or seeking diagnosis as at immediate risk of having, a colorectal neoplasm (e.g., colorectal cancer, advanced adenoma). In various instances, a human subject is a subjected identified as a subject in need of screening for (e.g., susceptible to) a colorectal neoplasm (e.g., colorectal cancer, advanced adenoma). In certain instances, a human subject is a subject identified as in need of colorectal cancer screening by a medical practitioner. In various instances, a human subject is identified as in need of colorectal cancer screening due to age, e.g., due to an age equal to or greater than 40 years, e.g., an age equal to or greater than 45, 50, 55, 60, 65, 70, 75, 80, 85, or 90 years, though in some instances a subject 18 years old or older may be identified as at risk and/or in need of screening for a colorectal neoplasm (e.g., colorectal cancer, advanced adenoma). In various instances, a human subject is identified as being high risk and/or in need of screening for a colorectal neoplasm (e.g., colorectal cancer, advanced adenoma) based on, without limitation, familial history, prior diagnoses, and/or an evaluation by a medical practitioner. In various instances, a human subject is a subject not diagnosed as having, not at risk of having, not at immediate risk of having, not diagnosed as having, and/or not seeking diagnosis for a cancer such as a colorectal cancer, or any combination thereof.

A sample from a subject, e.g., a human or other mammalian subject, can be a sample of, e.g., blood, blood component (e.g., plasma, buffy coat), cfDNA (cell free DNA), ctDNA (circulating tumor DNA), stool, or tissue (e.g., advanced adenoma and/or colorectal tissue). In some particular embodiments, a sample is an excretion or bodily fluid of a subject (e.g., stool, blood, plasma, lymph, or urine of a subject) or a tissue sample of a colorectal neoplasm, such as a colonic polyp, an advanced adenoma, and/or colorectal cancer. A sample from a subject can be a cell or tissue sample, e.g., a cell or tissue sample that is of a cancer or includes cancer cells, e.g., of a tumor or of a metastatic tissue. For example, the sample may include colorectal cells, polyp cells, or glandular cells. In various embodiments, a sample from a subject, e.g., a human or other mammalian subject, can be obtained by biopsy (e.g., colonoscopy resection, fine needle aspiration or tissue biopsy) or surgery.

In various particular embodiments, a sample is a sample of cell-free DNA (cfDNA). cfDNA is typically found in biological fluids (e.g., plasma, serum, or urine) in short, double-stranded fragments. The concentration of cfDNA is typically low, but can significantly increase under particular conditions, including without limitation pregnancy, autoimmune disorder, myocardial infraction, and cancer. Circulating tumor DNA (ctDNA) is the component of circulating DNA specifically derived from cancer cells. ctDNA can be present in human fluids. For example, in some instances, ctDNA can be found bound to and/or associated with leukocytes and erythrocytes. In some instances, ctDNA can be found not bound to and/or associated with leukocytes and erythrocytes. Various tests for detection of tumor-derived cfDNA are based on detection of genetic or epigenetic modifications that are characteristic of cancer (e.g., of a relevant cancer). Genetic or epigenetic modifications characteristic of cancer can include, without limitation, oncogenic or cancer-associated mutations in tumor-suppressor genes, activated oncogenes, hypermethylation, and/or chromosomal disorders. Detection of genetic or epigenetic modifications characteristic of cancer or pre-cancer can confirm that detected cfDNA is ctDNA.

cfDNA and ctDNA provide a real-time or nearly real-time metric of the methylation status of a source tissue. cfDNA and ctDNA have a half-life in blood of about 2 hours, such that a sample taken at a given time provides a relatively timely reflection of the status of a source tissue.

Various methods of isolating nucleic acids from a sample (e.g., of isolating cfDNA from blood or plasma) are known in the art. Nucleic acids can be isolated, e.g., without limitation, standard DNA purification techniques, by direct gene capture (e.g., by clarification of a sample to remove assay-inhibiting agents and capturing a target nucleic acid, if present, from the clarified sample with a capture agent to produce a capture complex, and isolating the capture complex to recover the target nucleic acid).

The amount of DNA (e.g., cfDNA) used in methods described herein is important to accurate determination of methylation status. In some embodiments, from about 8 ng to about 20 ng of DNA is required per sample (e.g., at least Ing, at least 8 ng, at least 10 ng, at least 15 ng, at least 20 ng). In certain embodiments, about 3 to about 4 mL of plasma is required to obtain a sufficient amount of cfDNA for sample processing.

### Methods of Measuring Methylation Status

Methylation status can be measured by a variety of methods known in the art and/or by methods provided in this specification. For example, in certain embodiments, one or more processing steps from U.S. application Ser. No. 17/744,231 filed on May 13, 2024, which is incorporated by reference in its entirety, may be used to determine methylation status of markers described herein. Those of skill in the art will appreciate that a method for measuring methylation status can generally be applied to samples from any source and of any kind, and will further be aware of processing steps available to modify a sample into a form suitable for measurement by a given methodology.

In certain embodiments, the processing steps involve fragmenting or shearing DNA of the sample. For example, genomic DNA (e.g., gDNA) obtained from a cell, tissue, or other source may require fragmentation prior to sequencing. In certain embodiments, DNA may be fragmented prior to measurement of methylation status using a physical method (e.g., using an ultra-sonicator, a nebulizer technique, hydrodynamic shearing, etc.). In certain embodiments, DNA may be fragmented using an enzymatic method (e.g., using an endonuclease or a transposase). Certain samples, e.g., cfDNA samples, may not require fragmentation. cfDNA fragments are about 100-200 bp in length and may be appropriate for certain methods provided herein. DNA fragments of about 100-1000 bp in length are suitable for analysis in certain NGS techniques described herein including, for example, Illumina® based techniques. Certain technologies may require DNA fragments of about 100-1000 bp range. In contrast, DNA fragments of about 10 kb or longer are suitable for long read sequencing technologies.

Methods of measuring methylation status include, without limitation, methods including whole genome bisulfite sequencing, targeted bisulfite sequencing, targeted enzymatic methylation sequencing, methylation-status-specific polymerase chain reaction (PCR), methods including mass spectrometry, methylation arrays, methods including methylation-specific nucleases, methods including mass-based separation, methods including target-specific capture (e.g., hybrid capture), and methods including methylation-specific oligonucleotide primers. Certain particular assays for methylation utilize a bisulfite reagent (e.g., hydrogen sulfite ions), methylation sensitive restriction enzymes, or enzymatic conversion reagents (e.g., Tet methylcytosine dioxygenase 2, T4 Phage β-glucosyltransferase, and APOBEC).

Bisulfite reagents can include, among other things, bisulfite, disulfite, hydrogen sulfite, sodium metabisulphite, or combinations thereof, which reagents can be useful in distinguishing methylated and unmethylated nucleic acids. Bisulfite interacts differently with cytosine and 5-methylcytosine. In typical bisulfite-based methods, contacting of DNA (e.g., single stranded DNA, double stranded DNA) with bisulfite deaminates (e.g., converts) unmethylated cytosine to uracil, while methylated cytosine remains unaffected. Methylated cytosines, but not unmethylated cytosines, are selectively retained. Thus, in a bisulfite processed sample, uracil residues stand in place of, and thus provide an identifying signal for, unmethylated cytosine residues, while remaining (methylated) cytosine residues thus provide an identifying signal for methylated cytosine residues. Bisulfite processed samples can be analyzed, e.g., by next generation sequencing (NGS) or other methods disclosed herein.

In some embodiments, bisulfite treatment includes subjecting DNA fragments (e.g., double stranded DNA) to one or more denaturation-conversion cycles in order to convert unmethylation cytosines to uracils in the DNA fragments. Denaturation converts double stranded DNA fragments in the sample to single stranded DNA fragments. In some embodiments, bisulfite treatment may be applied prior to library preparation. In some embodiments, bisfulfite treatment may be applied after library preparation.

Enzymatic conversion reagents can include Tet methylcytosine dioxygenase 2 (TET2) and T4 Phage β-glucosyltransferase (T4 BGT), among others. TET2 oxidizes 5-methylcytosine and thus protects it from the consecutive deamination by APOBEC. APOBEC deaminates unmethylated cytosine to uracil, while oxidized 5-methylcytosine remains unaffected. Thus, in a TET2 processed sample, uracil residues stand in place of, and thus provide an identifying signal for, unmethylated cytosine residues, while remaining (methylated) cytosine residues thus provide an identifying signal for methylated cytosine residues. TET2 processed samples can be analyzed, e.g., by next generation sequencing (NGS). In certain embodiments, APOBEC refers to a member (or plurality of members) of the Apolipoprotein B mRNA Editing Catalytic Polypeptide-like (APOBEC) family. In certain embodiments, APOBEC may refer to APOBEC-1, APOBEC-2, APOBEC-3A, APOBEC-3B, APOBEC-3C, APOBEC-3D, APOBEC-3E, APOBEC-3F, APOBEC-3G. APOBEC-3H, APOBEC-4, and/or Activation-induced (cytidine) deaminase (AID). In certain embodiments, enzymatic conversion reagents can include a glucosyltransferase (e.g., a β-glucosyltransferase (BGT), e.g., T4-phage β-glucosyltransferase (T4BGT)). T4 BGT transfers the glucose moiety of uridine diphosphoglucose (UDP-glucose) to the 5-hydroxymethylcytosine (5-hmC) residues in double-stranded DNA generating β-glucosyl-5 hydroxymethylcytosine, which aids in locus specific detection of 5-hmC residues and enrichment of 5-hmC containing DNA.

Methods of measuring methylation status can include, without limitation, massively parallel sequencing (e.g., next-generation sequencing) to determine methylation state, e.g., sequencing-by-synthesis, real-time (e.g., single-molecule) sequencing, bead emulsion sequencing, nanopore sequencing, or other sequencing techniques known in the art. In some embodiments, a method of measuring methylation status can include whole-genome sequencing, e.g., measuring whole genome methylation status from bisulfite or enzymatically treated material with base-pair resolution.

In some embodiments, a method of measuring methylation status includes reduced representation bisulfite sequencing e.g., utilizing use of restriction enzymes to measure methylation status of high CpG content regions from bisulfite or enzymatically treated material with base-pair resolution.

In some embodiments, a method of measuring methylation status can include targeted sequencing e.g., measuring methylation status of pre-selected genomic location from bisulfite or enzymatically treated material with base-pair resolution.

In some embodiments, the pre-selection (capture) (e.g., enrichment) of regions of interest (e.g., DMRs) can be done by complementary in vitro synthesized oligonucleotide sequences (e.g., capture baits/probes). Capture probes (e.g., oligonucleotide capture probes, oligonucleotide capture baits) are useful in targeted sequencing (e.g., NGS) techniques to enrich for particular regions of interest in an oligonucleotide (e.g., DNA) sequence. For example, enrichment of target regions is useful when sequences of particular pre-determined regions of DNA are sequenced. In certain embodiments, capture probes are about 10 to 1000 bp long (e.g., about 10 to about 200 bp long) (e.g., about 120 bp long). In certain embodiments, one or more capture probes are targeted to capture a region of interest (e.g., a genomic marker) corresponding to one or more methylation loci (e.g., methylation loci comprising at least a portion of one or more DMRs, e.g., as found in Table 1). In certain embodiments, capture probes are targeted to methylation loci that are hypomethylated or hypermethylated. For example, a capture probe may be targeted to particular methylation loci. However, if fragments of DNA corresponding to methylation loci are converted (e.g., bisulfite or enzymatic converted) prior to enrichment using a capture probe, the sequence of the converted DNA fragments will change as described herein due to particular cytosine residues being unmethylated. Therefore, targeting an unconverted DNA region may result in some mismatches if cytosines are hypomethylated. Though capture probe-target sequence hybridization may tolerate some mismatches, a second probe may be required to enrich for DNA regions which are hypomethylated.

In certain embodiments, the capture probes are nucleic acid probes (e.g., DNA probes, RNA probes). In some embodiments, a method may also include identifying mutated regions (e.g., individual nucleotide bases) using targeted sequencing e.g., determining the presence of a mutation in one or more pre-selected genomic locations (e.g., a genomic marker, e.g., a mutation marker). In certain embodiments, mutations may also be identified from bisulfite or enzymatically treated DNA with base-pair resolution.

In some embodiments, a method for measuring methylation status can include Illumina Methylation Assays e.g., measuring over 850,000 methylation sites quantitatively across a genome at single-nucleotide resolution.

Various methylation assay procedures can be used in conjunction with bisulfite treatment to determine methylation status of a target sequence such as a DMR. Such assays can include, among others, Methylation-Specific Restriction Enzyme qPCR, sequencing of bisulfite-treated nucleic acid, PCR (e.g., with sequence-specific amplification), Methylation Specific Nuclease-assisted Minor-allele Enrichment PCR, and Methylation-Sensitive High Resolution Melting. In some embodiments, DMRs are amplified from converted (e.g., bisulfite or enzyme converted) DNA fragments for library preparation.

In some embodiments, a sequencing library may be prepared using converted oligonucleotide fragments (e.g., fragments converted via an enzymatic conversion protocol), wherein the library is prepared, for example, using a TWIST cfDNA library preparation kit protocol, an IDT NGS library preparation protocol, or a modified Illumina or other sequencing platform-compatible library preparation protocol. In some embodiments, the oligonucleotide fragments are DNA fragments which have been converted (e.g., bisulfite or enzyme converted). In certain embodiments, DNA fragments used in preparation of a sequencing library may be single stranded DNA fragments or double stranded DNA fragments. In certain embodiments, a library may be prepared by attaching adapters to DNA fragments. Adapters contain short (e.g., about 100 to about 1000 bp) sequences (e.g., oligonucleotide sequences) that allow oligonucleotide fragments of a library (e.g., a DNA library) to bind to and generate clusters on a flow cell used in, for example, next generation sequencing (NGS). Adapters may be ligated to library fragments prior to NGS. In certain embodiments, a ligase enzyme covalently links the adapter and library fragments. In certain embodiments, adapters are attached to either one or both of the 5′ and 3′ ends of converted DNA fragments.

In certain embodiments, adapters are attached to DNA fragments prior to subjecting DNA to conversion. In certain embodiments, adapters used herein contain a sequence of oligonucleotides that aid in sample identification. For example, in certain embodiments, adapters include a sample index. A sample index is a short sequence (e.g., about 8 to about 10 bases) of nucleic acids (e.g., DNA, RNA) that serve as sample identifiers and allow for, among other things, multiplexing and/or pooling of multiple samples in a single sequencing run and/or on a flow cell (e.g., used in a NGS technique). In certain embodiments, an adapter at a 5′ end, a 3′ end, or both of a converted single stranded DNA fragment includes a sample index. In certain embodiments, an adapter sequence may include a molecular barcode. A molecular barcode may serve as a unique molecular identifier (UMI) to identify a target molecule during, for example, DNA sequencing. In certain embodiments, DNA barcodes may be randomly generated. In certain embodiments, DNA barcodes may be predetermined or predesigned. In certain embodiments, the DNA barcodes are different on each DNA fragment. In certain embodiments, the DNA barcodes may be the same for two single stranded DNA fragments that are not complementary to one another (e.g., in a Watson-Crick pair with each other) in the biological sample. In certain embodiments, DNA fragments may be amplified (e.g., using PCR) after ligation of adapters to DNA fragments. In certain embodiments, at least 40% (e.g., at least at least 50%, at least 60%, at least 70%) of the converted DNA fragments have an adapter attached at both the 5′ and 3′ ends.

In certain embodiments, high-throughput and/or next-generation sequencing (NGS) techniques are used to achieve base-pair level resolution of an oligonucleotide (e.g., a DNA) sequence, permitting analysis of methylation status and/or identification of mutations. For example, in certain embodiments, NGS may include single-end or paired-end sequencing. In single-end sequencing, a technique reads a sequenced fragment in one direction—from one end of a fragment to the opposite end of the fragment. In certain embodiments, this produces a single DNA sequence that then may be aligned to a reference sequence. In paired-end sequencing, a sequenced fragment is read in a first direction from one end of the fragment to the opposite end of the fragment. The sequenced fragment may be read until a specified read length is reached. Then, the sequenced fragment is read in a second direction, which is opposite to the first direction. In certain embodiments, having multiple read pairs may help to improve read alignment and/or identify mutations (e.g., insertions, deletions, inversion, etc.) that may not be detected by single-end reading.

Another method, that can be used for methylation detection includes PCR amplification with methylation-specific oligonucleotide primers (MSP methods), e.g., as applied to bisulfite-treated sample (see, e.g., Herman 1992 Proc. Natl. Acad. Sci. USA 93:9821-9826, which is herein incorporated by reference with respect to methods of determining methylation status). Use of methylation-status-specific oligonucleotide primers for amplification of bisulfite-treated DNA allows differentiation between methylated and unmethylated nucleic acids. Oligonucleotide primer pairs for use in MSP methods include at least one oligonucleotide primer capable of hybridizing with sequence that includes a methylation site, e.g., a CpG site. An oligonucleotide primer that includes a T residue at a position complementary to a cytosine residue will selectively hybridize to templates in which the cytosine was unmethylated prior to bisulfite treatment, while an oligonucleotide primer that includes a G residue at a position complementary to a cytosine residue will selectively hybridize to templates in which the cytosine was methylated cytosine prior to bisulfite treatment. MSP results can be obtained with or without sequencing amplicons, e.g., using gel electrophoresis. MSP (methylation-specific PCR) allows for highly sensitive detection (detection level of 0.1% of the alleles, with full specificity) of locus-specific DNA methylation, using PCR amplification of bisulfite-converted DNA.

Another method that can be used to determine methylation status after bisulfite treatment of a sample is Methylation-Sensitive High Resolution Melting (MS-HRM) PCR (see, e.g., Hussmann 2018 Methods Mol Biol. 1708:551-571, which is herein incorporated by reference with respect to methods of determining methylation status). MS-HRM is an in-tube, PCR-based method to detect methylation levels at specific loci of interest based on hybridization melting. Bisulfite treatment of the DNA prior to performing MS-HRM ensures a different base composition between methylated and unmethylated DNA, which is used to separate the resulting amplicons by high resolution melting. A unique primer design facilitates a high sensitivity of the assays enabling detection of down to 0.1-1% methylated alleles in an unmethylated background. Oligonucleotide primers for MS-HRM assays are designed to be complementary to the methylated allele, and a specific annealing temperature enables these primers to anneal both to the methylated and the unmethylated alleles thereby increasing the sensitivity of the assays.

Another method that can be used to determine methylation status after bisulfite treatment of a sample is Quantitative Multiplex Methylation-Specific PCR (QM-MSP). QM-MSP uses methylation specific primers for sensitive quantification of DNA methylation (see, e.g., Fackler 2018 Methods Mol Biol. 1708:473-496, which is herein incorporated by reference with respect to methods of determining methylation status). QM-MSP is a two-step PCR approach, where in the first step, one pair of gene-specific primers (forward and reverse) amplifies the methylated and unmethylated copies of the same gene simultaneously and in multiplex, in one PCR reaction. This methylation-independent amplification step produces amplicons of up to 10⁹ copies per μL after 36 cycles of PCR. In the second step, the amplicons of the first reaction are quantified with a standard curve using real-time PCR and two independent fluorophores to detect methylated/unmethylated DNA of each gene in the same well (e.g., 6FAM and VIC). One methylated copy is detectable in 100,000 reference gene copies.

Another method that can be used to determine methylation status after bisulfite treatment of a sample is Methylation Specific Nuclease-assisted Minor-allele Enrichment (MS-NaME) (see, e.g., Liu 2017 Nucleic Acids Res. 45 (6): e39, which is herein incorporated by reference with respect to methods of determining methylation status). Ms-NaME is based on selective hybridization of probes to target sequences in the presence of DNA nuclease specific to double-stranded (ds) DNA (DSN), such that hybridization results in regions of double-stranded DNA that are subsequently digested by the DSN. Thus, oligonucleotide probes targeting unmethylated sequences generate local double stranded regions resulting to digestion of unmethylated targets; oligonucleotide probes capable of hybridizing to methylated sequences generate local double-stranded regions that result in digestion of methylated targets, leaving methylated targets intact. Moreover, oligonucleotide probes can direct DSN activity to multiple targets in bisulfite-treated DNA, simultaneously. Subsequent amplification can enrich non-digested sequences. Ms-NaME can be used, either independently or in combination with other techniques provided herein.

Another method that can be used to determine methylation status after bisulfite treatment of a sample is Methylation-sensitive Single Nucleotide Primer Extension (Ms-SNuPE™) (see, e.g., Gonzalgo 2007 Nat Protoc. 2 (8): 1931-6, which is herein incorporated by reference with respect to methods of determining methylation status). In Ms-SNuPE, strand-specific PCR is performed to generate a DNA template for quantitative methylation analysis using Ms-SNuPE. SNuPE is then performed with oligonucleotide(s) designed to hybridize immediately upstream of the CpG site(s) being interrogated. Reaction products can be electrophoresed on polyacrylamide gels for visualization and quantitation by phosphor-image analysis. Amplicons can also carry a directly or indirectly detectable labels such as a fluorescent label, radionuclide, or a detachable molecule fragment or other entity having a mass that can be distinguished by mass spectrometry. Detection may be carried out and/or visualized by means of, e.g., matrix assisted laser desorption/ionization mass spectrometry (MALDI) or using electron spray mass spectrometry (ESI).

Certain methods that can be used to determine methylation status after bisulfite treatment of a sample utilize a first oligonucleotide primer, a second oligonucleotide primer, and an oligonucleotide probe in an amplification-based method. For instance, the oligonucleotide primers and probe can be used in a method of real-time polymerase chain reaction (PCR) or droplet digital PCR (ddPCR). In various instances, the first oligonucleotide primer, the second oligonucleotide primer, and/or the oligonucleotide probe selectively hybridize methylated DNA and/or unmethylated DNA, such that amplification or probe signal indicate methylation status of a sample.

Other bisulfite-based methods for detecting methylation status (e.g., the presence of level of 5-methylcytosine) are disclosed, e.g., in Frommer (1992 Proc Natl Acad Sci USA. 1; 89 (5): 1827-31, which is herein incorporated by reference with respect to methods of determining methylation status).

In certain MSRE-qPCR embodiments, the amount of total DNA is measured in an aliquot of sample in native (e.g., undigested) form using, e.g., real-time PCR or digital PCR.

Various amplification technologies can be used alone or in conjunction with other techniques described herein for detection of methylation status. Those of skill in the art, having reviewed the present specification, will understand how to combine various amplification technologies known in the art and/or described herein together with various other technologies for methylation status determination known in the art and/or provided herein. Amplification technologies include, without limitation, PCR, e.g., quantitative PCR (qPCR), real-time PCR, and/or digital PCR. Those of skill in the art will appreciate that polymerase amplification can multiplex amplification of multiple targets in a single reaction. PCR amplicons are typically 100 to 2000 base pairs in length. In various instances, an amplification technology is sufficient to determine methylations status.

Digital PCR (dPCR) based methods involve dividing and distributing a sample across wells of a plate with 96-, 384-, or more wells, or in individual emulsion droplets (ddPCR) e.g., using a microfluidic device, such that some wells include one or more copies of template and others include no copies of template. Thus, the average number of template molecules per well is less than one prior to amplification. The number of wells in which amplification of template occurs provides a measure of template concentration. If the sample has been contacted with MSRE, the number of wells in which amplification of template occurs provides a measure of the concentration of methylated template.

In various embodiments a fluorescence-based real-time PCR assay, such as MethyLight™, can be used to measure methylation status (see, e.g., Campan 2018 Methods Mol Biol. 1708:497-513, which is herein incorporated by reference with respect to methods of determining methylation status). MethyLight is a quantitative, fluorescence-based, real-time PCR method to sensitively detect and quantify DNA methylation of candidate regions of the genome. MethyLight is uniquely suited for detecting low-frequency methylated DNA regions against a high background of unmethylated DNA, as it combines methylation-specific priming with methylation-specific fluorescent probing. Additionally, MethyLight can be combined with Digital PCR, for the highly sensitive detection of individual methylated molecules, with use in disease detection and screening.

Real-time PCR-based methods for use in determining methylation status typically include a step of generating a standard curve for unmethylated DNA based on analysis of external standards. A standard curve can be constructed from at least two points and can permit comparison of a real-time Ct value for digested DNA and/or a real-time Ct value for undigested DNA to known quantitative standards. In particular instances, sample Ct values can be determined for MSRE-digested and/or undigested samples or sample aliquots, and the genomic equivalents of DNA can be calculated from the standard curve. Ct values of MSRE-digested and undigested DNA can be evaluated to identify amplicons digested (e.g., efficiently digested; e.g., yielding a Ct value of 45). Amplicons not amplified under either digested or undigested conditions can also be identified. Corrected Ct values for amplicons of interest can then be directly compared across conditions to establish relative differences in methylation status between conditions. Alternatively or additionally, delta-difference between the Ct values of digested and undigested DNA can be used to establish relative differences in methylation status between conditions.

In certain particular embodiments, targeted bisulfite or enzymatic sequencing (e.g., using hybrid capture) among other techniques, can be used to determine the methylation status of a methylation biomarker for a disease and/or condition. For example, a colorectal neoplasm (e.g., advanced adenoma and/or colorectal cancer) methylation biomarker that is or includes a single methylation locus. In certain particular embodiments, targeted bisulfite sequencing, among other techniques, can be used to determine the methylation status of a methylation biomarker that is or includes two or more methylation loci.

Those of skill in the art will appreciate that in embodiments in which a plurality of methylation loci (e.g., a plurality of DMRs) are analyzed for methylation status in a method of screening for colorectal cancer provided herein, methylation status of each methylation locus can be measured or represented in any of a variety of forms, and the methylation statuses of a plurality of methylation loci (preferably each measured and/or represented in a same, similar, or comparable manner) be together or cumulatively analyzed or represented in any of a variety of forms. In various embodiments, the methylation status of each methylation locus can be measured as a methylation portion. In various embodiments, methylation status of each methylation locus can be represented as the percentage value of methylated reads from total sequencing reads compared against reference sample. In various embodiments, methylation status of each methylation locus can be represented as a qualitative comparison to a reference, e.g., by identification of each methylation locus as hypermethylated or hypomethylated.

In some embodiments in which a single methylation locus is analyzed, hypermethylation of the single methylation locus constitutes a diagnosis that a subject is suffering from or possibly suffering from a condition (e.g., cancer) (e.g., advanced adenoma, colorectal cancer), while absence of hypermethylation of the single methylation locus constitutes a diagnosis that the subject is likely not suffering from a condition. In some embodiments, hypermethylation of a single methylation locus (e.g., a single DMR) of a plurality of analyzed methylation loci constitutes a diagnosis that a subject is suffering from or possibly suffering from the condition, while the absence of hypermethylation at any methylation locus of a plurality of analyzed methylation loci constitutes a diagnosis that a subject is likely not suffering from the condition. In some embodiments, hypermethylation of a determined percentage (e.g., a predetermined percentage) of methylation loci (e.g., at least 10% (e.g., at least 10%, at least 20%, at least 30%, at least 40%, at least 50%, at least 60%, at least 70%, at least 80%, at least 90%, or 100%)) of a plurality of analyzed methylation loci constitutes a diagnosis that a subject is suffering from or possibly suffering from the condition, while the absence of hypermethylation of a determined percentage (e.g., a predetermined percentage) of methylation loci (e.g., at least 10% (e.g., at least 10%, at least 20%, at least 30%, at least 40%, at least 50%, at least 60%, at least 70%, at least 80%, at least 90%, or 100%)) of a plurality of analyzed methylation loci constitutes a diagnosis that a subject is not likely suffering from the condition. In some embodiments, hypermethylation of a determined number (e.g., a predetermined number) of methylation loci (e.g., at least 1, 2, 3, 4, 5, 6, 7, 8, 9, 10, 11, 12 or more DMRs) of a plurality of analyzed methylation loci (e.g. 2, 3, 4, 5, 6, 7, 8, 9, 10, 11, 12 or more DMRs) constitutes a diagnosis that a subject is suffering from or possibly suffering from the condition, while the absence of hypermethylation of a determined number (e.g., a predetermined number) of methylation loci (e.g., at least 1, 2, 3, 4, 5, 6, 7, 8, 9, 10, 11, 12, or more DMRs) of a plurality of analyzed methylation loci (e.g., 2, 3, 4, 5, 6, 7, 8, 9, 10, 11, 12 or more DMRs) constitutes a diagnosis that a subject is not likely suffering from the condition.

In some embodiments, methylation status of a plurality of methylation loci (e.g., a plurality of DMRs) is measured qualitatively or quantitatively and the measurement for each of the plurality of methylation loci are combined to provide a diagnosis. In some embodiments, the quantitatively measured methylation status of each of a plurality of methylation loci is individually weighted, and weighted values are combined to provide a single value that can be comparative to a reference in order to provide a diagnosis.

In some embodiments, methylation status may include determination of methylated and/or unmethylated reads mapped to a genomic region (e.g., a DMR). For example, when using particular sequencing technologies as disclosed herein (e.g., NGS, whole genome bisulfite sequencing, etc.), sequence reads are produced. A sequence read is an inferred sequence of base pairs (e.g., a probabilistic sequence) corresponding to all or part of a sequenced oligonucleotide (e.g., DNA) fragment (e.g., cfDNA fragments, gDNA fragments). In certain embodiments, sequence reads may be mapped (e.g., aligned) to a particular, pre-determined region of interest using a reference sequence (e.g., a bisulfite or enzymatic converted reference sequence) in order to determine if there are any alterations or variations in a read. Alterations may include methylation and/or mutations. A region of interest may include one or more genomic markers including a methylation marker (e.g., a DMR), a mutation marker, or other marker as disclosed herein. In certain embodiments, the region of interest at least 100, at least 200, at least 300, at least 400, at least 500, at least 600, at least 700, at least 800, at least 900, at least 1000 base pairs upstream and/or downstream of the at least one of the one or more markers (e.g., DMRs).

For example, in the case of bisulfite or enzymatically treated DNA fragments, treatment converts unmethylated cytosines to uracils, while methylated cytosines are not converted to uracils. Accordingly, a sequence read produced for a DNA fragment that has methylated cytosines will be different from a sequence read produced for the same DNA fragment that does not have methylated cytosine. Methylation at sites where a cytosine nucleotide is followed by a guanine nucleotide (e.g., CpG sites) may be of particular interest.

In certain embodiments, a region of interest may be sequenced to a read depth of at least 100×, at least 200×, at least 300×, at least 400×, at least 500×, at least 600×, at least 700×, at least 800×, at least 900×, at least 1000× or greater. Read depth is the number of times each individual base within a region has been sequenced.

### Identifying Mutations

In certain embodiments as disclosed herein, genomic mutations may be identified in one or more predetermined mutation biomarkers, for example, as disclosed in U.S. application Ser. No. 17/744,231 filed on May 13, 2024, which is incorporated by reference in its entirety. In various embodiments, a mutation biomarker of the present disclosure is used for further detection (e.g., screening) and/or classification of a condition in addition to methylation biomarkers. In certain embodiments, information regarding a methylation status of one or more colorectal cancer biomarkers may be combined with a mutation biomarker in order to further classify the identified colorectal cancer. In addition or alternatively, mutation biomarkers may be used to determine or recommend (e.g., either for or against) a particular course of treatment for the identified disease and/or condition.

In certain embodiments, identifying genomic mutations may be performed using a sequencing technique as discussed herein (e.g., a NGS sequencing technique). In certain embodiments, oligonucleotides (e.g., cfDNA fragments, gDNA fragments) are sequenced to a read depth sufficient to detect a genomic mutation (e.g., in a mutation biomarker, in a tumor markers) at a frequency in a sample as low as 1.0%, 0.75%, 0.5%, 0.25%, 0.1%, 0.075%, 0.05%, 0.025%, 0.01%, or 0.005%.

Genomic mutations generally include any variation in nucleotide base pair sequences of DNA as is understood in the art. A mutation in a nucleic acid may, in some embodiments, include a single nucleotide variant, an inversion, a deletion, an insertion, a transversion, a translocation, a fusion, a truncation, an amplification, or a combination thereof, as compared to a reference DNA sequence.

Mutations may be identified using NGS sequencing techniques (e.g., targeted NGS sequencing techniques, hybridization NGS sequencing techniques, or the like) or other sequencing techniques disclosed herein. In certain embodiments as disclosed herein, mutations may be identified in converted (e.g., bisulfite or enzymatic converted) DNA fragments. In certain embodiments, mutations and methylated loci may be identified in parallel (e.g., simultaneously) using a single sequencing assay (e.g., an NGS assay). In certain embodiments, one or more capture probes are targeted to capture and/or enrich for a region of interest of an oligonucleotide (e.g., DNA) sequence corresponding to one or more mutations markers.

### Artificial Spike-in Control

In certain embodiments, artificial spike-in controls control nucleic acid (e.g., DNA) molecules (e.g., “spike-in controls”) as described in U.S. application Ser. No. 17/744,231 filed on May 13, 2024, which is incorporated by reference in its entirety, are used to evaluate or estimate conversion efficiency of unmethylated and methylated cytosines to uracils. Control nucleic acid molecules may be used in sequencing methods involving conversion (e.g., bisulfite or enzymatic conversion) of DNA samples. When DNA is subjected to conversion (e.g., bisulfite or enzymatic conversion) as described herein, conversion may be incomplete. That is, some number of unmethylated cytosines may not be converted to uracils. If the conversion is not complete such that unmethylated cytosines are not mostly converted, the unconverted unmethylated cytosines may be identified as methylated when the DNA sequenced. Accordingly, in order to determine whether or not conversion is complete, a control DNA molecule may be subjected to conversion along with DNA fragments from a sample. In certain embodiments, sequencing the converted control DNA molecules (e.g., using an NGS technique as described herein) generates a plurality of control sequence reads. Control sequence reads may be used to determine conversion rates of unmethylated and/or methylated cytosines to uracils.

The conversion rate of unmethylated cytosines to uracils in DNA fragments may vary significantly from on sample to another. For example, conversion efficiency may range from 10% to 110% within a single batch of processed samples. Note, there can be over-conversion such that conversion efficiency can be greater than 100%, e.g., the conversion efficiency is 110% when 10% of the methylated cytosine gets converted. In certain embodiments, the conversion efficiency ranges from 30% to 110%. In other embodiments, the conversion efficiency ranges from 50% to 100%.

In certain embodiments, a control DNA molecule may be added to a sample after fragmentation and before conversion using e.g., bisulfite or enzymatic reagents. In certain embodiments, a plurality (e.g., two, three, four or more) control DNA sequences may be added to DNA fragments of a sample. A control DNA molecule may be a known sequence. For example, the sequence, number of methylated bases, and number of unmethylated bases of the control sequence had been determined prior to addition of the control DNA molecule to the sample. In certain embodiments, a control sequence may be a DNA sequence which is produced in vitro to contain artificially methylated or unmethylated nucleotides (e.g., methylated cytosines). In certain embodiments, a control sequence may be a DNA sequence which is produced to contain completely unmethylated DNA nucleotides.

A high conversion efficiency of the spike-in control sequence may be used to infer the conversion efficiency of a DNA fragments undergoing the same conversion process as a spike-in control. For example, deamination of at least at least 98% of unmethylated cytosines in the unmethylated spike-in control DNA sequence indicates that conversion efficiency is high and that a sample may pass a quality control assessment. In certain embodiments, at least 90%, at least 95%, at least 96%, at least 97%, at least 98%, at least 99% of unmethylated cytosines of a plurality of DNA fragments of a control DNA sequence are converted into uracils. A high conversion efficiency is important as it is ideal for all (or nearly all) of the unmethylated cytosines to be converted to uracils when subjecting DNA to bisulfite or enzymatic treatments. As described above, unconverted, unmethylated cytosines may serve as a source of noise in the data.

In addition, conversion of methylated cytosines to uracils is undesirable when DNA is treated using a conversion process. Conversion of methylated cytosines of a spike-in control is indicative that methylated cytosines have been converted to uracils in a DNA sample subjected to the same treatment as the methylated spike-in control. Methylated cytosines in a methylated spike-in control should not convert to uracils. For the same reasons as described above, methylated cytosines being converted to uracils may result in misidentification of purportedly unmethylated cytosines during methylation analysis. In certain embodiments, at most 5%, at most 4%, at most 3%, at most 2% or at most 1% of methylated cytosines of a plurality of DNA fragments of a control DNA sequence are converted into uracils. For example, deamination of at most 2% of methylated cytosines in a methylated spike-in control DNA sequence indicates that conversion efficiency is high and that a sample may pass a quality control assessment.

### Exemplary Deduplication Steps

In certain embodiments as discussed herein and described in U.S. application Ser. No. 17/744,231 filed on May 13, 2024, which is incorporated by reference in its entirety, duplicate sequences are found in sequencing data. Duplicate sequences arise from a number of potential sources, and accordingly may need to be removed from sequencing data. Duplicates are particularly important to remove in an analysis as signals from cancer are low. Cancer signals would get lost in noise if duplicates are not removed.

For example, in certain embodiments sequencing data may include a large number of reads obtained from sequencing oligonucleotide fragments (e.g., DNA fragments, e.g., cfDNA, gDNA fragments) of a sample. Multiple reads corresponding to a particular DNA fragment may result in false variant calls (e.g., identification of multiple variants of the same DNA fragment), which would interfere with the identification of a methylated CpG site and/or a mutation. In certain embodiments, duplicate sequences are removed prior to determining read-wise methylation values. In certain embodiments, a bioinformatics package (e.g., Picard, SAMTools) may be used to mark and remove duplicates from sequencing data.

In certain embodiments, PCR duplicates (also known as library duplicates) and/or over-sequencing duplicates may also be removed. PCR duplicates and over-sequencing duplicates are sequence reads that result from sequencing two or more copies of the exact same DNA fragment. PCR duplicates and over-sequencing duplicates may arise during library preparation. In certain embodiments, sequence reads are considered PCR duplicates or over-sequencing duplicates if the sequence reads have (1) a 5′ end coordinate (i.e., a start position), (2) a 3′ end coordinate (i.e., an end position), and (3) a methylation level that are the same, wherein the 5′ end coordinate and the 3′ end coordinate of a sequence read correspond to the position at which the 5′-most nucleotide and the 3′-most nucleotide, respectively, of the sequence read map to a reference sequence. Finally, the deduplicated reads are quality filtered (1350), which results in the removal of additional reads.

In certain embodiments, deduplicating sequence reads does not comprise removing duplicate sequence reads that have a different methylation level. For example, a sample may have two sequence reads that are identical. However, one sequence read may have a CpG site that is methylated, while the same CpG site in the other strand is not methylated. In certain embodiments, both strands may be kept for further bioinformatics analysis. Without wishing to be bound to any particular theory, a presence of different methylation levels within duplicate fragments may be due to sequencing errors or a different source of one fragment.

### Applications

Methods and compositions of the present disclosure can be used in any of a variety of applications. For example, methods and compositions of the present disclosure can be used to screen, or aid in screening for a condition (e.g., cancer). In particular, the methods and compositions can be used to screen, or aid in screening for a colorectal neoplasm, e.g., advanced adenoma and/or colorectal cancer. For example, in certain embodiments, any one or more of (i), (ii), and (iii) may apply: (i) the methods and/or compositions are useful in the detection of colorectal cancer; (ii) in certain embodiments, the methods and/or compositions are useful in the detection of advanced adenoma; and/or (iii) in certain embodiments, the methods and/or compositions are useful in the undifferentiated detection of colorectal cancer or advanced adenoma.

In various instances, screening using methods and compositions of the present disclosure can detect any stage of colorectal cancer, including without limitation early-stage colorectal cancer. For example, in certain embodiments, the techniques and compositions described herein are useful for the detection of a particular stage of colorectal cancer (for example, one of the stages described in the “Colorectal Cancers” section above), and in certain embodiments, the techniques and compositions described herein are useful for undifferentiated detection of the presence of any of two or more stages (e.g., the determination that the subject has either Stage 1 or Stage 2 colorectal cancer (or, in other examples, other groups of two or more particular stages), but not necessarily a determination as to which stage the subject has). In some embodiments, screening using methods and compositions of the present disclosure is applied to individuals 40 years of age or older, e.g., 40, 45, 50, 55, 60, 65, 70, 75, 80, 85, or 90 years or older. In particular, individuals 40 years of age or older are of interest for colorectal cancer and/or advanced adenoma screening. In some embodiments, screening using methods and compositions of the present disclosure is applied to individuals 18 years of age or older, e.g., 18, 20, 25, 30, 35, 40, 45, 50, 55, 60, 65, 70, 75, 80, 85, or 90 years or older. In some embodiments, screening using methods and compositions of the present disclosure is applied to individuals 18 to 40 years of age. In various embodiments, screening using methods and compositions of the present disclosure is applied to individuals experiencing abdominal pain or discomfort, e.g., experiencing undiagnosed or incompletely diagnosed abdominal pain or discomfort. In various embodiments, screening using methods and compositions of the present disclosure is applied to individuals experiencing no symptoms likely to be associated with a cancer or a colorectal neoplasm such as advanced adenoma, polyposis, and/or colorectal cancer. Thus, in certain embodiments, screening using methods and compositions of the present disclosure is fully or partially preventative or prophylactic, at least with respect to later or non-early stages of cancer.

In various embodiments, cancer screening using methods and compositions of the present disclosure can be applied to an asymptomatic human subject. In particular, a subject can be referred to as “asymptomatic” if the subject does not report, and/or demonstrate by non-invasively observable indicia (e.g., without one, several, or all of device-based probing, tissue sample analysis, bodily fluid analysis, surgery, or cancer screening), sufficient characteristics of the condition to support a medically reasonable suspicion that the subject is likely suffering from the condition. Detection of a colorectal neoplasm such as advanced adenoma and/or early stage colorectal cancer is particularly likely in asymptomatic individuals screened in accordance with methods and compositions of the present disclosure.

Those of skill in the art will appreciate that regular, preventative, and/or prophylactic screening for a colorectal neoplasm such as advanced adenoma and/or colorectal cancer improves diagnosis. As noted above, early stage cancers include, according to at least one system of cancer staging, Stages 0 to II C of colorectal cancer. Thus, the present disclosure provides, among other things, methods and compositions particularly useful for the diagnosis and treatment of colorectal neoplasms including advanced adenoma, polyposis and/or early stage colorectal cancer. Generally, and particularly in embodiments in which screening in accordance with the present disclosure is carried out annually, and/or in which a subject is asymptomatic at time of screening, methods and compositions of the present invention are especially likely to detect early stage colorectal cancer.

In various embodiments colorectal cancer screening in accordance with the present disclosure is performed once for a given subject or multiple times for a given subject. In various embodiments, colorectal cancer screening in accordance with the present disclosure is performed on a regular basis, e.g., every six months, annually, every two years, every three years, every four years, every five years, or every ten years.

In various embodiments, screening using methods and compositions disclosed herein will provide a diagnosis of a condition (e.g., a type or class of a colorectal neoplasm). In other instances, screening for colorectal neoplasms using methods and compositions disclosed herein will be indicative of having one or more conditions, but not definitive for diagnosis of a particular condition. For example, screening may be used to classify a subject as having one or more conditions or combination of conditions including, but not limited to, advanced adenoma and/or colorectal cancer. In various instances, screening using methods and compositions of the present disclosure can be followed by a further diagnosis-confirmatory assay, which further assay can confirm, support, undermine, or reject a diagnosis resulting from prior screening, e.g., screening in accordance with the present disclosure.

In various embodiments, screening in accordance with methods and compositions of the present disclosure reduces colorectal cancer mortality, e.g., by early colorectal cancer diagnosis. Data supports that colorectal cancer screening reduces colorectal cancer mortality, which effect persisted for over 30 years (see, e.g., Shaukat 2013 N Engl J Med. 369 (12): 1106-14). Moreover, colorectal cancer is particularly difficult to treat at least in part because colorectal cancer, absent timely screening, may not be detected until cancer is past early stages. For at least this reason, treatment of colorectal cancer is often unsuccessful. To maximize population-wide improvement of colorectal cancer outcomes, utilization of screening in accordance with the present disclosure can be paired with, e.g., recruitment of eligible subjects to ensure widespread screening.

In various embodiments, screening of colorectal neoplasms including one or more methods and/or compositions disclosed herein is followed by treatment of colorectal cancer. In various embodiments, treatment of colorectal cancer, includes administration of a therapeutic regimen including one or more of surgery, radiation therapy, and chemotherapy. In various embodiments, treatment of colorectal cancer, e.g., early stage colorectal cancer, includes administration of a therapeutic regimen including one or more of treatments provided herein for treatment of stage 0 colorectal cancer, stage I colorectal cancer, and/or stage II colorectal cancer.

In various embodiments, treatment of colorectal cancer includes treatment of early stage colorectal cancer, e.g., stage 0 colorectal cancer or stage I colorectal cancer, by one or more of surgical removal of cancerous tissue e.g., by local excision (e.g., by colonoscope), partial colectomy, or complete colectomy.

In various embodiments, treatment of colorectal cancer includes treatment of early stage colorectal cancer, e.g., stage II colorectal cancer, by one or more of surgical removal of cancerous tissue (e.g., by local excision (e.g., by colonoscope), partial colectomy, or complete colectomy), surgery to remove lymph nodes near to identified colorectal cancer tissue, and chemotherapy (e.g., administration of one or more of 5-FU and leucovorin, oxaliplatin, or capecitabine).

In various embodiments, treatment of colorectal cancer includes treatment of stage III colorectal cancer, by one or more of surgical removal of cancerous tissue (e.g., by local excision (e.g., by colonoscopy-based excision), partial colectomy, or complete colectomy), surgical removal of lymph nodes near to identified colorectal cancer tissue, chemotherapy (e.g., administration of one or more of 5-FU, leucovorin, oxaliplatin, capecitabine, e.g., in a combination of (i) 5-FU and leucovorin, (ii) 5-FU, leucovorin, and oxaliplatin (e.g., FOLFOX), or (iii) capecitabine and oxaliplatin (e.g., CAPEOX)), and radiation therapy.

In various embodiments, treatment of colorectal cancer includes treatment of stage IV colorectal cancer, by one or more of surgical removal of cancerous tissue (e.g., by local excision (e.g., by colonoscope), partial colectomy, or complete colectomy), surgical removal of lymph nodes near to identified colorectal cancer tissue, surgical removal of metastases, chemotherapy (e.g., administration of one or more of 5-FU, leucovorin, oxaliplatin, capecitabine, irinotecan, VEGF-targeted therapeutic agent (e.g., bevacizumab, ziv-aflibercept, or ramucirumab), EGFR-targeted therapeutic agent (e.g., cetuximab or panitumumab), Regorafenib, trifluridine, and tipiracil, e.g., in a combination of or including (i) 5-FU and leucovorin, (ii) 5-FU, leucovorin, and oxaliplatin (e.g., FOLFOX), (iii) capecitabine and oxaliplatin (e.g., CAPEOX), (iv) leucovorin, 5-FU, oxaliplatin, and irinotecan (FOLFOXIRI), and (v) trifluridine and tipiracil (Lonsurf)), radiation therapy, hepatic artery infusion (e.g., if cancer has metastasized to liver), ablation of tumors, embolization of tumors, colon stent, colorectomy, colostomy (e.g., diverting colostomy), and immunotherapy (e.g., pembrolizumab).

Those of skill in the art that treatments of colorectal cancer provided herein can be utilized, e.g., as determined by a medical practitioner, alone or in any combination, in any order, regimen, and/or therapeutic program. Those of skill in the art will further appreciate that advanced treatment options may be appropriate for earlier stage cancers in subjects previously having suffered a cancer or colorectal cancer, e.g., subjects diagnosed as having a recurrent colorectal cancer.

In some embodiments, methods and compositions for colorectal neoplasm screening provided herein can inform treatment and/or payment (e.g., reimbursement for or reduction of cost of medical care, such as screening or treatment) decisions and/or actions, e.g., by individuals, healthcare facilities, healthcare practitioners, health insurance providers, governmental bodies, or other parties interested in healthcare cost.

In some embodiments, methods and compositions for colorectal neoplasm screening provided herein can inform decision making relating to whether health insurance providers reimburse a healthcare cost payer or recipient (or not), e.g., for (1) screening itself (e.g., reimbursement for screening otherwise unavailable, available only for periodic/regular screening, or available only for temporally- and/or incidentally-motivated screening); and/or for (2) treatment, including initiating, maintaining, and/or altering therapy, e.g., based on screening results. For example, in some embodiments, methods and compositions for colorectal neoplasm screening provided herein are used as the basis for, to contribute to, or support a determination as to whether a reimbursement or cost reduction will be provided to a healthcare cost payer or recipient. In some instances, a party seeking reimbursement or cost reduction can provide results of a screen conducted in accordance with the present specification together with a request for such reimbursement or cost reduction of a healthcare cost. In some instances, a party making a determination as to whether or not to provide a reimbursement or cost reduction of a healthcare cost will reach a determination based in whole or in part upon receipt and/or review of results of a screen conducted in accordance with the present specification.

For the avoidance of any doubt, those of skill in the art will appreciate from the present disclosure that methods and compositions for colorectal cancer diagnosis of the present specification are at least for in vitro use. Accordingly, all aspects and embodiments of the present disclosure can be performed and/or used at least in vitro.

### EXEMPLIFICATION

In order that the application may be more fully understood, the following examples are set forth. It should be understood that these examples are for illustrative purposes only and are not to be construed as limiting in any manner.

### Example 1

The present example provides data relating to the performance of individual DMRs used in detection (e.g., diagnosis) and stratification of colorectal cancer.

The methylation status of each of the DMRs listed in Table 1 herein was used to detect and stratify colorectal cancer (CRC). 38 samples from patients with stage IV CRC, 46 samples from patients with stage III CRC, 89 samples from patients with stage II CRC, and 28 samples from patients with stage I CRC were used to evaluate the performance of each DMR in detection and stratification. Accordingly, a total of 201 samples from subjects having colorectal cancer were used in testing. Additional control samples from subjects without colorectal cancer were used when determining the specificity of the DMRs in detecting colorectal cancer.

For each DMR, a random forest (RF) machine learning algorithm was used to receive information regarding the methylation status of an individual DMR and subsequently determine the stage and/or the presence of colorectal cancer. The specificity and sensitivity for each DMR were calculated based on the algorithm's output.

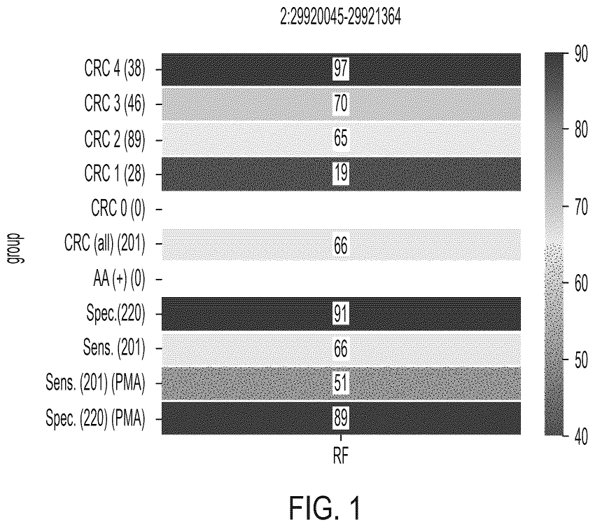

For example, FIG. 1 shows data relating to the sensitivity and specificity of the DMR chr2: 29920045-29921364 (SEQ ID NO. 1). The DMR is a stable Tango region and can be located using the provided information in the GRCh38 build of the human genome. A stable region is a region that showed a similar performance across different technical units. A stage I region is a region that maximizes performance in Stage I (e.g., not necessarily a stable region). In FIG. 1, the sensitivity of the DMR in detecting stage IV (CRC 4) colorectal cancer was 97%, the sensitivity in detecting stage III (CRC 3) colorectal cancer was 70%, the sensitivity in detecting stage II (CRC 2) colorectal cancer was 65%, and the sensitivity in detecting stage I (CRC 1) colorectal cancer was 19%. The overall sensitivity in detecting colorectal cancer was 66% (CRC (all) and Sens.). The specificity for detecting colorectal cancer was 91%. PMA (Premarket Approval) referred to in FIG. 1 is a metric related to a weighted score of sensitivity and specificity. Sensitivity PMA can be weighted by the distribution of cancer stages (with higher weights given to earlier stage cancers). Specificity PMA is weighted by the age distribution of subjects (with higher weights given to subjects with ages ranging from 65 years to 69 years).

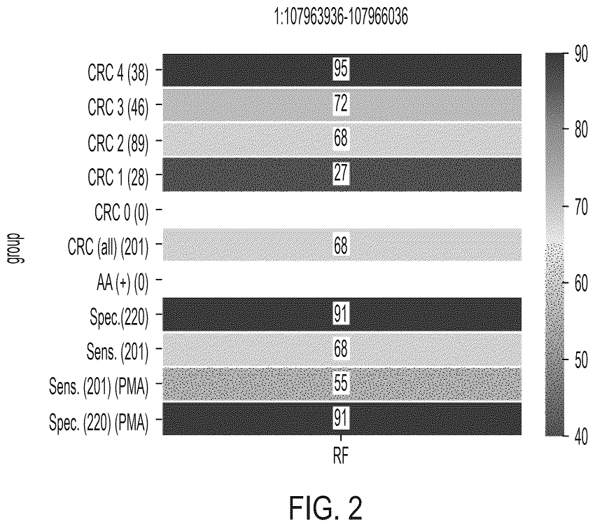

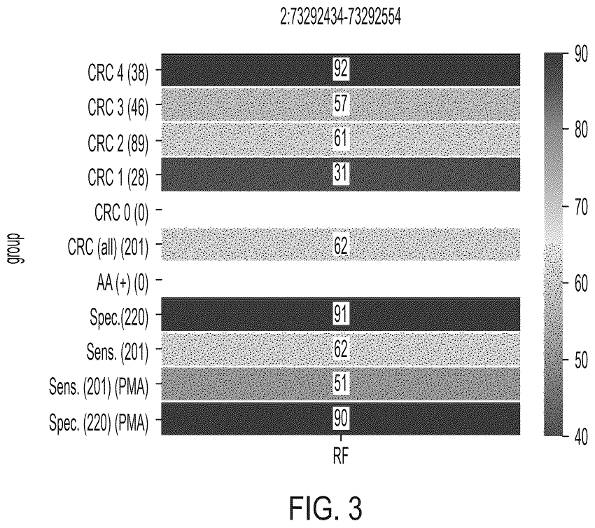

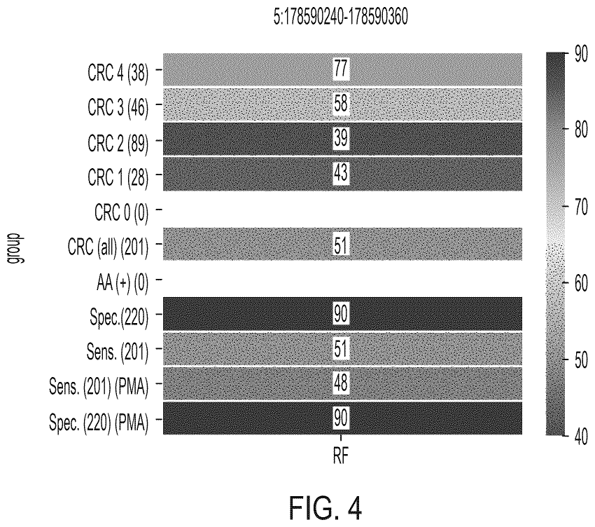

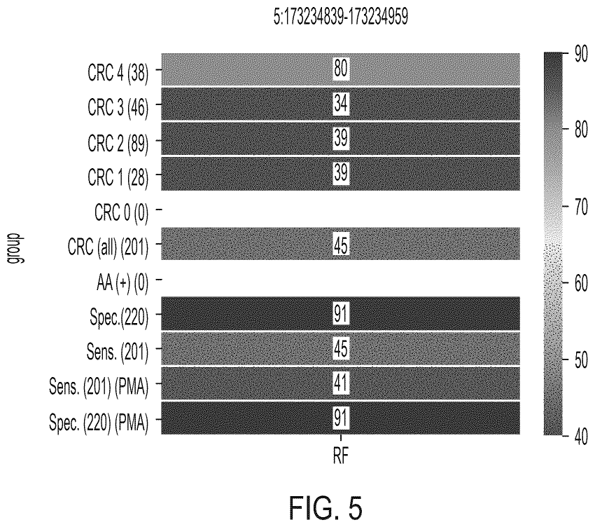

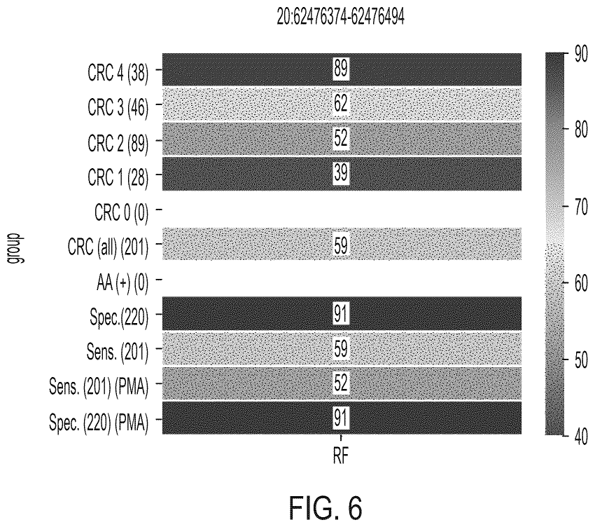

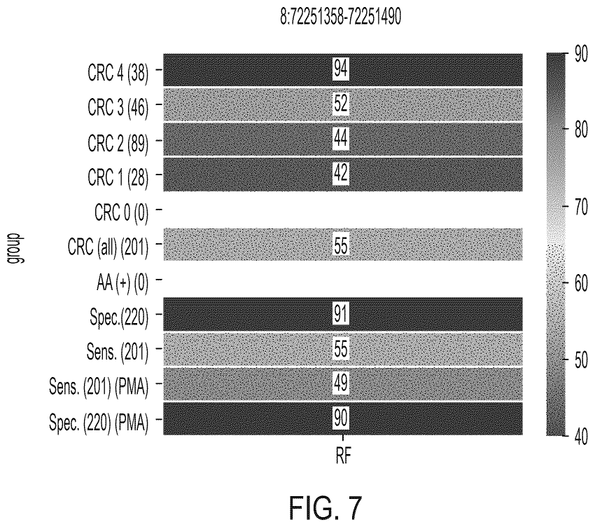

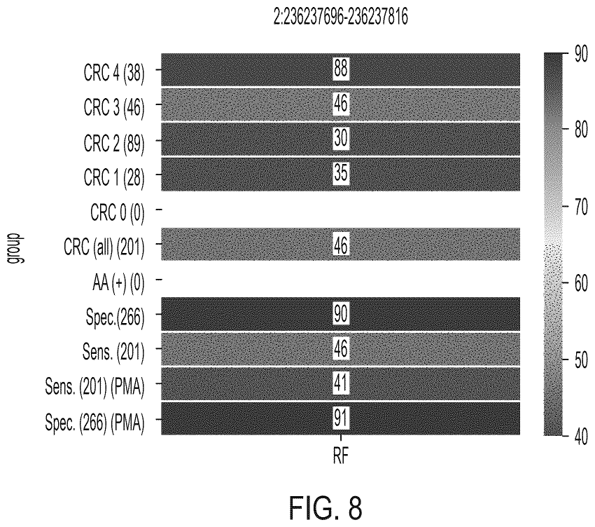

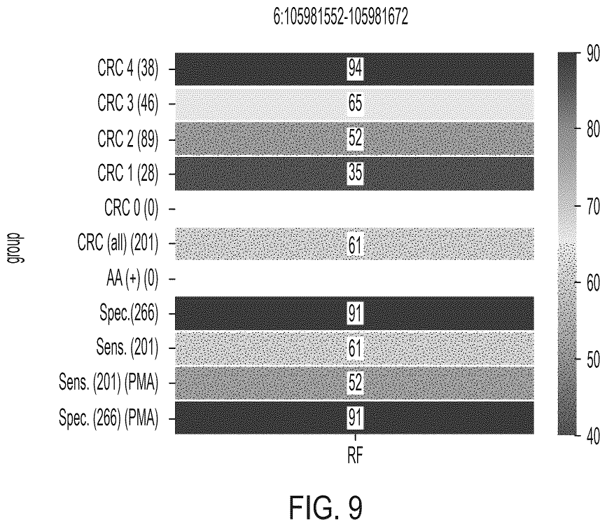

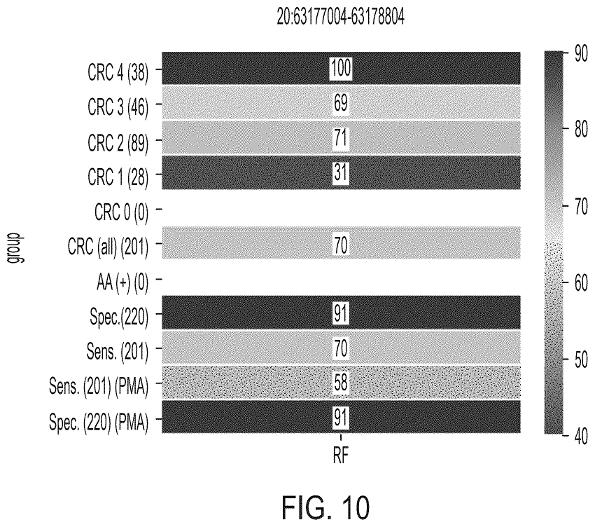

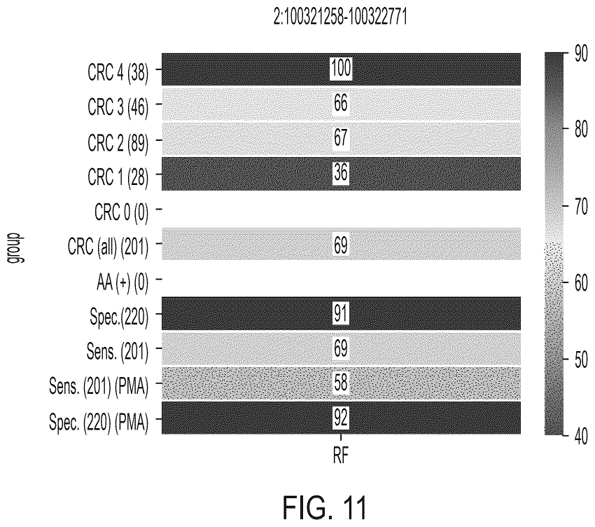

Similar to FIG. 1, FIGS. 2-12 show data relating to the sensitivity and specificity of the remaining DMRs from Table 1. Each DMR was tested using total of 201 samples from subjects having colorectal cancer with additional controls added for specificity testing. DMRs chr5: 178590240-178590360 (SEQ ID NO. 4), chr5: 173234839-173234959 (SEQ ID NO. 5), chr20: 62476374-62476494 (SEQ ID NO. 6), chr8: 72251358-72251490 (SEQ ID NO. 7), chr2: 236237696-236237816 (SEQ ID NO. 8), and chr6: 105981552-105981672 (SEQ ID NO. 9) were noted as being Stage I CRC best performing regions. DMRs chr2: 29920045-29921364 (SEQ ID NO. 1), chr1: 107963936-107966036 (SEQ ID NO. 2), and chr2: 73292434-73292554 (SEQ ID NO. 3) were noted as stable Tango regions. DMRs chr1: 107963936-107966036 (SEQ ID NO. 2), chr20: 63177004-63178804 (SEQ ID NO. 10), chr2: 100321258-100322771 (SEQ ID NO. 11), and chr4: 143699944-143701144 (SEQ ID NO. 12) were noted as being best performing regions.

### Example 2

Among other things, the present example shows methods for identification of detecting methylation statuses of markers and determining which markers are useful in determining the presence of colorectal cancer and its stages in patients suffering from colorectal cancer as compared to controls.

### Identification of Markers

In particular, experiments of the present Example examined best performing regions in samples from colorectal cancers of 217 subjects and colons of 221 healthy control subjects not diagnosed as suffering from colorectal cancer.

Samples were analyzed using an algorithm that searches for cancer-specific methylation signatures in plasma cfDNA from read-wise (horizontal) methylation patterns. The method counts all the reads that have the following properties:
1) The reads overlap a region of interest (e.g., a DMR).
2) The reads have 80-100% methylation of CpGs inside of the region of interest.
3) The reads have a certain number of CpGs in the region of interest. In the present experiment, each of the reads had at least 4 CpGs in the region of interest and up to 27 CpGs in the region of interest. The number of methylated reads corresponding to the region of interest were normalized this number by all reads that overlap the region.

During a training phase on separate, training data, the algorithm learns to filter reads based on, among other things, the number of CpGs in the below regions. Below are the learned bins for each of the regions. Table 11 below shows ranges of CpGs in reads for the identified regions.

TABLE 11
CpG region bins and combination of DMRs.

| Region | | |
|---|---|---|
| (Chromosome: start-end) | SEQ ID NO. | CpG filter |
| chr2: 100321258-100322771 | 11 | [4, 25] CpGs in read |
| chr1: 107963936-107966036 | 2 | [4, 27] CpGs in read |
| chr20: 63177004-63178804 | 10 | [8, 26] CpGs in read |
| chr20: 62476374-62476494 | 6 | [4, 17] CpGs in read |

Discovery of markers relevant to colorectal cancer identification involved making a list of eligible regions using the following eligibility criteria:
1) High Feature Importance in Machine Learning models (using Monte Carlo Cross Validation) for sample classification,
2) High Univariate classification performance for certain subgroups of samples (e.g., CRC Stage I, CRC Stage IIA, etc.),
3) Observed high stability across sequencing batches (or plates or experiments) of high feature importance of machine learning models.

Region discovery was followed by identifying combinations of regions that give the best performance for colorectal cancer detection. Table 12 shown below shows exemplary regions which met the above eligibility criteria. For the avoidance of doubt, each of the identified markers are individually useful in identifying colorectal cancer and its stages.

TABLE 12
Regions and Discovery Criteria.

| Region | SEQ | |
|---|---|---|
| (Chromosome: start-end) | ID NO. | Discovery Criteria |
| chr2: 100321258-100322771 | 11 | A best performing region in machine learning |
| | | (ML) models |
| | | Univariate classification performance was |
| | | among the best for all stages of CRC and CRC |
| | | Stage I |
| chr1: 107963936-107966036 | 2 | A best performing region in ML models |
| | | Univariate best for all CRC and CRC IIA |
| | | Among stablest of regions |
| chr20: 63177004-63178804 | 10 | A best performing region in ML models |
| | | Univariate classification performance was |
| | | among the best for all CRC and CRC IIA |
| chr20: 62476374-62476494 | 6 | Univariate classification performance was |
| | | among the best for CRC I |
| | | Among stablest of regions |

### Evaluation of Marker Performance

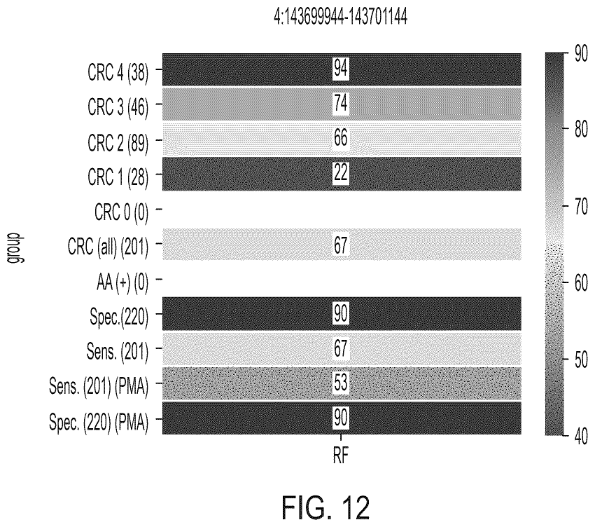

When evaluated individually, region L (chr4: 143699944-143701144, SEQ ID NO. 12) was found to be the most effective region (e.g., as shown in FIG. 12).

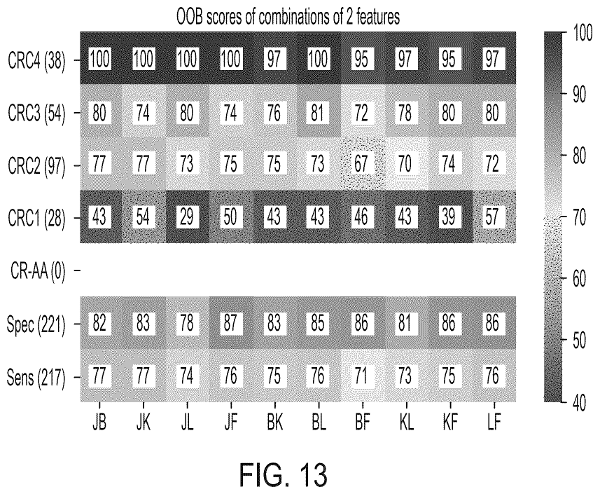

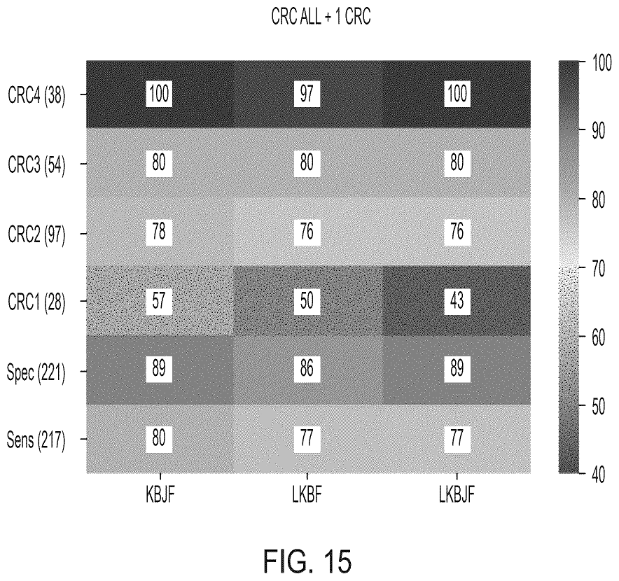

Subsequently, different combinations of regions were tested to determine which regions worked better for identifying colorectal cancer and its various types. FIGS. 13-16 show the OOB (out-of-bag) scores for combinations of two to five regions from Table 13, along with the sensitivity and specificity of the marker combinations. Table 13 below shows annotations corresponding to individual regions along with the corresponding region of the genome where the sequence is found, the SEQ ID NO, and discovery criteria that warranted its inclusion in the below list. Features listed below were found to be among the best performing in identifying CRC over all its stages (CRC all), among the best markers for identifying stage I CRC (Stage I CRC), or among the best at identifying advanced adenoma (CR-AA).

TABLE 13
Listing of 7 DMRs.

| Region | SEQ | Discovery | |
|---|---|---|---|
| (Chromosome: start-end) | ID NO. | Criteria | Annotation |
| chr4: 143699944-143701144 | 12 | CRC all | L |
| chr2: 100321258-100322771 | 11 | CRC all | K |
| chr1: 107963936-107966036 | 2 | CRC all | B |
| chr20: 63177004-63178804 | 10 | CRC all | J |
| chr8: 72251358-72251490 | 7 | Stage I CRC | G |
| chr20: 62476374-62476494 | 6 | Stage I CRC | F |
| chr5: 132073832-132073952 | 13 | CR-AA | M |

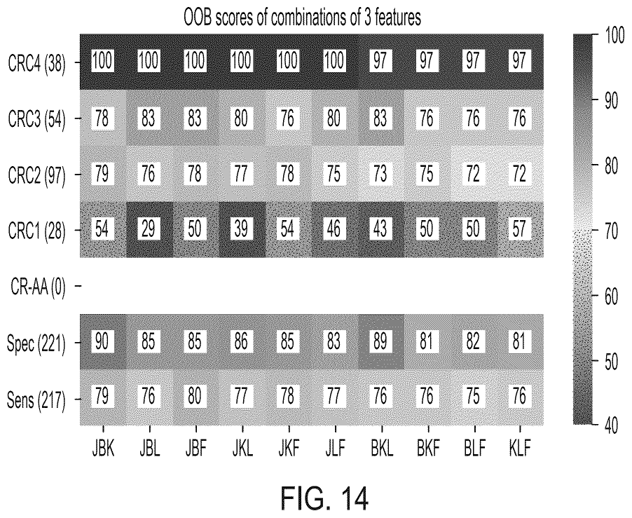

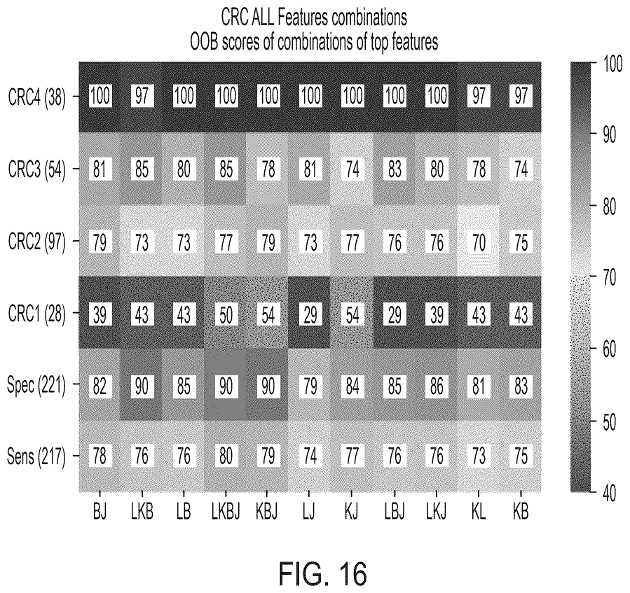

When testing the performance of different combinations of two regions (e.g., as shown in FIGS. 13 and 16), it was found that combinations JK [chr20: 63177004-63178804 (SEQ ID NO. 10), chr2: 100321258-100322771 (SEQ ID NO. 11)] and JB [chr20: 63177004-63178804 (SEQ ID NO. 10), chr1: 107963936-107966036 (SEQ ID NO. 2)] were the best performing combinations of two regions. Performance of two region combinations was improved by adding a third region (e.g., as shown in FIG. 14). The combination JKB [chr20: 63177004-63178804 (SEQ ID NO. 10), chr2: 100321258-100322771 (SEQ ID NO. 11), chr1: 107963936-107966036 (SEQ ID NO. 2)] provided improved performance over combinations JK and JB. An additional region, region F provided the best performing combination of regions-JBKF [chr20: 63177004-63178804 (SEQ ID NO. 10), chr1: 107963936-107966036 (SEQ ID NO. 2), chr2: 100321258-100322771 (SEQ ID NO. 11), chr20: 62476374-62476494 (SEQ ID NO. 6)]. Region F [chr20: 62476374-62476494 (SEQ ID NO. 6)] is a region which is relevant to CRC stage I identification. As shown in, for example, FIG. 16, combinations LKB, KBJ, LKBJ were found to be the most effective combinations together, with best combination being KBJ. For example, the combination KBJF was found to perform better than the combinations LKB, KBJ, and LKBJ.

Thus, the present Example generated a set of four (KBJF) methylation regions that when combined, serve as a methylation biomarker for colorectal cancer detection. Each of the four specified regions was found to be hypermethylated in colorectal cancer as compared to healthy controls.

### Example 3

Among other things, the present example describes an exemplary process for identification (e.g., detecting) methylation status of one or more markers as described herein.

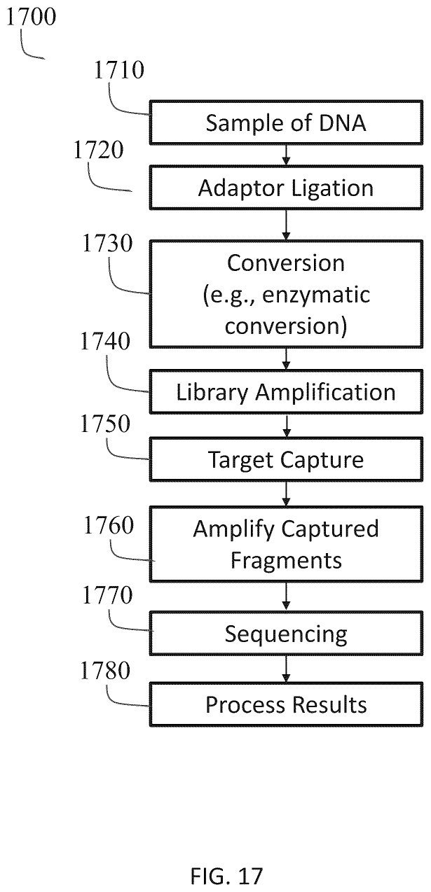

FIG. 17 shows a process (1700) for obtaining methylation statuses of markers. In the process (1700) shown in FIG. 17, a sample of DNA (e.g., cell-free DNA) is obtained (1710). The amount of cell-free DNA used in the process is an important factor in obtaining accurate results. About 3 mL to about 4 mL of plasma is isolated from two tubes of 5 mL to 10 mL of whole blood collection. This yields about 1 to 20 ng of cell-free DNA per sample.

Next, adaptors are ligated to cfDNA fragments in the sample (1720). Illumina adaptors are used, which includes indexes (i.e., barcodes) to help identify the source of cfDNA fragments.

After adapter ligation (1720), the DNA fragments undergo conversion using an enzymatic conversion process (1730). In the enzymatic conversion process, Tet methylcytosine dioxygenase 2 (TET2) and T4-phage β-glucosyltransferase are used to protect methylated sites in the DNA fragments. Apolipoprotein B mRNA editing catalytic polypeptide-like (APOBEC) proteins are then used to convert the unprotected, unmethylated cytosines to uracil in the DNA fragments.

After conversion (1730), the library of DNA fragments created through the prior processing steps are amplified (1740).

Target capture (1750) is then performed on the amplified library. For each run, a pool of 8 patient libraries (i.e., from different samples) is typically used. However, fewer or more libraries may be pooled together. The use of pooled DNA is helpful in reducing nonspecific binding.

In the target capture step (1750), probes targeting regions of interest (i.e., bait probes) are hybridized to cfDNA fragments in the pooled DNA libraries. The bait probes are designed to capture fully methylated and fully unmethylated regions of interest. The use of probes to capture both fully methylated and fully unmethylated regions of interest (e.g., methylation markers, e.g., DMRs) allow for more complete capture of target DNA regions. The temperature and time for hybridization has been found to be important to the hybridization process. Bait probes hybridized to cfDNA fragments are then bound to streptavidin beads, thereby adhering cfDNA fragments with regions of interests indirectly to the beads.

Post-hybridization, samples are washed at 65° C. to remove unbound DNA fragments. The bound DNA fragments are subsequently detached from the beads and amplified (1760) using primers.

The bound DNA fragments that are released from the beads are then sequenced (1770) to obtain reads of the cfDNA fragments. From 1 to 48 8-plexed pools can be sequenced on a single lane in a flow cell. However, typically, 12 8-plexed pools (for a total of 96 samples) are sequenced per lane. The post-sequencing coverage of target regions can be 50× or higher, as determined after filtering and processing.

Processing the sequencing results (1780) involves alignment, deduplication, length checking, and quality filtering of reads obtained from cfDNA fragments from patients and controls. The processing of aligning reads involves the use of BWAMeth and Bismark. Reads are aligned (i.e., mapped) to pre-determined regions within a reference genome. The pre-determined regions are regions of interest that include methylation marker(s) of interest (e.g., a DMR) and 1000 bp on each side of the methylation marker(s).

Deduplication is also performed on the aligned reads for both samples and control sequences. Deduplication is performed based on the start position of the read, the end position of the read, and the methylation level (i.e., the methylation status of bases within the read, e.g., the methylation status of CpG sites within the read).

During processing, the length of the reads are also checked to, among other things, determine the degree of coverage of sequencing.

Quality filtering is then performed to remove poor-quality reads that failed quality check criteria.

After the reads are quality filtered, samples are analyzed using an algorithm that searches for cancer-specific methylation signatures in plasma cfDNA from read-wise (horizontal) methylation patterns (e.g., as discussed herein). Single nucleotide polymorphisms (SNPs) and smaller indels are also assessed.

The cancer-specific methylation signature is then provided to a machine learning algorithm (e.g., as described herein) to identify signal corresponding to cancer.

### Computer System and Network Environment

Certain embodiments described herein make use of computer algorithms (e.g., machine learning (ML) algorithms and/or non-ML algorithms) in the form of software instructions executed by a computer processor. For example, computer algorithms including logistic regression algorithms (“Logistic”), ensemble learning algorithms such as XGBoost (“XGB-el-optimized”), voting ensembles (“Voting new”), and random forest (“RF”) machine learning algorithms (e.g., RF-50c-3s) can be used in accordance with embodiments of the present disclosure. The specificity and sensitivity for each of the one or more markers can be calculated based on the algorithm's output. In some embodiments, data can be collected and/or analyzed by each plate of tested sample(s). In some embodiments, data can be collected and/or analyzed by each batch of tested sample(s).

In certain embodiments, the software instructions include a machine learning (ML) module. As used herein, a machine learning (ML) module refers to a computer implemented process (e.g., a software function) that implements one or more specific machine learning techniques (e.g., as described herein) such as a neural network (e.g., a deep neural network), logistic regression algorithms, ensemble learning algorithms such as XGBoost (e.g., XGB-el-optimized which is a boosting model that builds trees sequentially), voting ensembles, and RF machine learning algorithms {e.g., an RF machine learning algorithm that is a bagging model (e.g., bootstrap aggregating) that build all decision trees simultaneously} or the like for a given input in order to provide, for a given input, one or more output values. In certain embodiments, a computer algorithm described herein is used to classify a patient as having a condition (e.g., colorectal cancer, advanced adenoma). In some embodiments, a subject is classified as having a condition based on, at least, a methylation status (e.g., a methylation value) of one or more markers (e.g., DMRs).

In certain embodiments, machine learning modules implementing machine learning techniques are trained, for example using datasets that include categories of data described herein (e.g., sequencing data, DNA methylation data). Such training may be used to determine various parameters of machine learning algorithms implemented by a machine learning module, such as weights associated with layers in neural networks. In certain embodiments, once a machine learning module is trained, the machine learning module is used to process new data (e.g., different from the training data) and accomplish its trained task without further updates to its parameters (e.g., the machine learning module does not receive feedback and/or updates). In certain embodiments, two or more machine learning modules may be combined and implemented as a single module and/or a single software application. In certain embodiments, two or more machine learning modules may also be implemented separately, e.g., as separate software applications. A machine learning module may be software and/or hardware. For example, a machine learning module may be implemented entirely as software, or certain functions of a module may be carried out via specialized hardware (e.g., via an application specific integrated circuit (ASIC)).

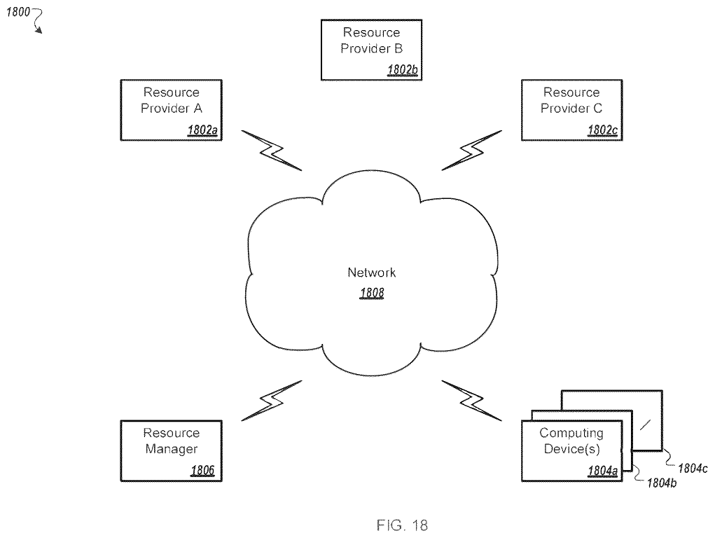

As shown in FIG. 18, an implementation of a network environment 1800 for use in providing systems, methods, and architectures for identifying biomarkers for detection of a disease or condition such as advanced adenoma, colorectal cancer, other cancers, or other diseases or conditions associated with an aberrant methylation status as described herein is shown and described. In brief overview, referring now to FIG. 18, a block diagram of an exemplary cloud computing environment 1800 is shown and described. The cloud computing environment 1800 may include one or more resource providers 1802a, 1802b, 1802c (collectively, 1802). Each resource provider 1802 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 1802 may be connected to any other resource provider 1802 in the cloud computing environment 1800. In some implementations, the resource providers 1802 may be connected over a computer network 1808. Each resource provider 1802 may be connected to one or more computing device 1804a, 1804b, 1804c (collectively, 1804), over the computer network 1808.

The cloud computing environment 1800 may include a resource manager 1806. The resource manager 1806 may be connected to the resource providers 1802 and the computing devices 1804 over the computer network 1808. In some implementations, the resource manager 1806 may facilitate the provision of computing resources by one or more resource providers 1802 to one or more computing devices 1804. The resource manager 1806 may receive a request for a computing resource from a particular computing device 1804. The resource manager 1806 may identify one or more resource providers 1802 capable of providing the computing resource requested by the computing device 1804. The resource manager 1806 may select a resource provider 1802 to provide the computing resource. The resource manager 1806 may facilitate a connection between the resource provider 1802 and a particular computing device 1804. In some implementations, the resource manager 1806 may establish a connection between a particular resource provider 1802 and a particular computing device 1804. In some implementations, the resource manager 1806 may redirect a particular computing device 1804 to a particular resource provider 1802 with the requested computing resource.

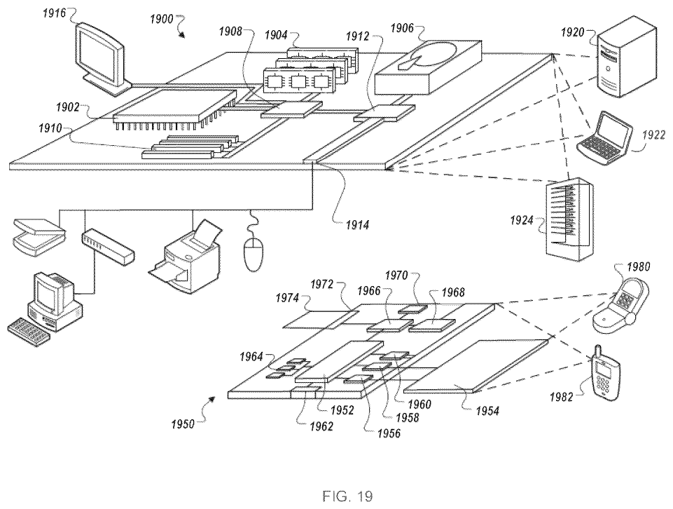

FIG. 19 shows an example of a computing device 1900 and a mobile computing device 1950 that can be used to implement the techniques described in this disclosure. The computing device 1900 is intended to represent various forms of digital computers, such as laptops, desktops, workstations, personal digital assistants, servers, blade servers, mainframes, and other appropriate computers. The mobile computing device 1950 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 1900 includes a processor 1902, a memory 1904, a storage device 1906, a high-speed interface 1908 connecting to the memory 1904 and multiple high-speed expansion ports 1910, and a low-speed interface 1912 connecting to a low-speed expansion port 1914 and the storage device 1906. Each of the processor 1902, the memory 1904, the storage device 1906, the high-speed interface 1908, the high-speed expansion ports 1910, and the low-speed interface 1912, are interconnected using various busses, and may be mounted on a common motherboard or in other manners as appropriate. The processor 1902 can process instructions for execution within the computing device 1900, including instructions stored in the memory 1904 or on the storage device 1906 to display graphical information for a GUI on an external input/output device, such as a display 1916 coupled to the high-speed interface 1908. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (one or more) of any number of computing devices (one or more). Furthermore, where a function is described as being performed by “a processor”, this encompasses embodiments wherein the function is performed by any number of processors (one or more) of any number of computing devices (one or more) (e.g., in a distributed computing system).

The memory 1904 stores information within the computing device 1900. In some implementations, the memory 1904 is a volatile memory unit or units. In some implementations, the memory 1904 is a non-volatile memory unit or units. The memory 1904 may also be another form of computer-readable medium, such as a magnetic or optical disk.

The storage device 1906 is capable of providing mass storage for the computing device 1900. In some implementations, the storage device 1906 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 1902), perform one or more methods, such as those described above. The instructions can also be stored by one or more storage devices such as computer- or machine-readable mediums (for example, the memory 1904, the storage device 1906, or memory on the processor 1902).

The high-speed interface 1908 manages bandwidth-intensive operations for the computing device 1900, while the low-speed interface 1912 manages lower bandwidth-intensive operations. Such allocation of functions is an example only. In some implementations, the high-speed interface 1908 is coupled to the memory 1904, the display 1916 (e.g., through a graphics processor or accelerator), and to the high-speed expansion ports 1910, which may accept various expansion cards (not shown). In the implementation, the low-speed interface 1912 is coupled to the storage device 1906 and the low-speed expansion port 1914. The low-speed expansion port 1914, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 1900 may be implemented in a number of different forms, as shown in the figure. For example, it may be implemented as a standard server 1920, or multiple times in a group of such servers. In addition, it may be implemented in a personal computer such as a laptop computer 1922. It may also be implemented as part of a rack server system 1917. Alternatively, components from the computing device 1900 may be combined with other components in a mobile device (not shown), such as a mobile computing device 1950. Each of such devices may contain one or more of the computing device 1900 and the mobile computing device 1950, and an entire system may be made up of multiple computing devices communicating with each other.

The mobile computing device 1950 includes a processor 1952, a memory 1964, an input/output device such as a display 1954, a communication interface 1966, and a transceiver 1968, among other components. The mobile computing device 1950 may also be provided with a storage device, such as a micro-drive or other device, to provide additional storage. Each of the processor 1952, the memory 1964, the display 1954, the communication interface 1966, and the transceiver 1968, are interconnected using various buses, and several of the components may be mounted on a common motherboard or in other manners as appropriate.

The processor 1952 can execute instructions within the mobile computing device 1950, including instructions stored in the memory 1964. The processor 1952 may be implemented as a chipset of chips that include separate and multiple analog and digital processors. The processor 1952 may provide, for example, for coordination of the other components of the mobile computing device 1950, such as control of user interfaces, applications run by the mobile computing device 1950, and wireless communication by the mobile computing device 1950.

The processor 1952 may communicate with a user through a control interface 1958 and a display interface 1956 coupled to the display 1954. The display 1954 may be, for example, a TFT (Thin-Film-Transistor Liquid Crystal Display) display or an OLED (Organic Light Emitting Diode) display, or other appropriate display technology. The display interface 1956 may comprise appropriate circuitry for driving the display 1954 to present graphical and other information to a user. The control interface 1958 may receive commands from a user and convert them for submission to the processor 1952. In addition, an external interface 1962 may provide communication with the processor 1952, so as to enable near area communication of the mobile computing device 1950 with other devices. The external interface 1962 may provide, for example, for wired communication in some implementations, or for wireless communication in other implementations, and multiple interfaces may also be used.

The memory 1964 stores information within the mobile computing device 1950. The memory 1964 can be implemented as one or more of a computer-readable medium or media, a volatile memory unit or units, or a non-volatile memory unit or units. An expansion memory 1974 may also be provided and connected to the mobile computing device 1950 through an expansion interface 1972, which may include, for example, a SIMM (Single In Line Memory Module) card interface. The expansion memory 1974 may provide extra storage space for the mobile computing device 1950, or may also store applications or other information for the mobile computing device 1950. Specifically, the expansion memory 1974 may include instructions to carry out or supplement the processes described above, and may include secure information also. Thus, for example, the expansion memory 1974 may be provide as a security module for the mobile computing device 1950, and may be programmed with instructions that permit secure use of the mobile computing device 1950. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier such that the instructions, when executed by one or more processing devices (for example, processor 1952), perform one or more methods, such as those described above. The instructions can also be stored by one or more storage devices, such as one or more computer- or machine-readable mediums (for example, the memory 1964, the expansion memory 1974, or memory on the processor 1952). In some implementations, the instructions can be received in a propagated signal, for example, over the transceiver 1968 or the external interface 1962.

The mobile computing device 1950 may communicate wirelessly through the communication interface 1966, which may include digital signal processing circuitry where necessary. The communication interface 1966 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 1968 using a radio-frequency. In addition, short-range communication may occur, such as using a Bluetooth®, Wi-Fi™, or other such transceiver (not shown). In addition, a GPS (Global Positioning System) receiver module 1970 may provide additional navigation- and location-related wireless data to the mobile computing device 1950, which may be used as appropriate by applications running on the mobile computing device 1950.

The mobile computing device 1950 may also communicate audibly using an audio codec 1960, which may receive spoken information from a user and convert it to usable digital information. The audio codec 1960 may likewise generate audible sound for a user, such as through a speaker, e.g., in a handset of the mobile computing device 1950. Such sound may include sound from voice telephone calls, may include recorded sound (e.g., voice messages, music files, etc.) and may also include sound generated by applications operating on the mobile computing device 1950.

The mobile computing device 1950 may be implemented in a number of different forms, as shown in the figure. For example, it may be implemented as a cellular telephone 1980. It may also be implemented as part of a smart-phone 1982, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein.

Throughout the description, where apparatus and systems are described as having, including, or comprising specific components, or where processes and methods are described as having, including, or comprising specific steps, it is contemplated that, additionally, there are apparatus, and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

While the invention has been particularly shown and described with reference to specific preferred embodiments, it should be understood by those skilled in the art that various changes in form and detail may be made therein without departing from the spirit and scope of the invention as defined by the appended claims.

SEQUENCES

chr2: 29920045-29921364 (SEQ ID NO. 1)
GGACAGCCTTCCCTCTCTGCCCACTTCCGACGCCTTCTTCTCGGGCATCAGGCGGATC
CTCAGTCGCCCTTCGCCTTGGCGAATCCACCAACTGAACAGCTCGCTGAGATTGAAC
TGGAGCAGCCCCACAGCCGCCTCCCCGGGGGGCCCGACGCAACCCTCCAAGATCGC
CTCCTCGCCCAGCTCCAGCACCAACTGCTTGGCACGCCGGAGCTTGCGCACGGAGCC
GCCCTTCAGCACCCTGGACAGCGTCCGGGCCTCTGCCGGGGCTGGTGAACCGGCGG
TCCAGGAGACCCCCGGCGCCGGCCCCAGCAACCTGAGCAGCGGGGCGCAGTCCAGA
GCTAGCGAGCCGCGGGCCTCGGGCCTGCCAGCCTTCAGCTCCGAGGAGGATGGTGG
CAGCAGTAGGTCCCGGGCGTAGACACGGAAGAGCGAGGGCACCACGAAGTCAACTG
CCAGACTCTTCCTCTGCAGGCGCGAGTAGCTGAGTGGCTCCCGGGGCTGCAGCGGCG
GCCCCGCAGCTGGGGAGCCCGCGCGCTGGCCGGTCCCCATCCCGGAGCCCACAGCT
GCCGTGGAAAGCAGCAGCGGCAGGAGCCACAGGAGCCCGATGGCTCCCATCCCGCC
GGAGGAGGCCGTTTACACTGCTCTCCGGGCCCAGCCTCACCCTTCGCTCTCCCCGAG
ATGGGAAGAGGCTCTGAACAGTCCTTGGTACCCAGCGGCTCCTTCCACCTGATCTCC
AGAGGACTGTGCGTGCGCGCAAGTCTCTTGCTTTCCCCCAACTGCACGGAGGCGAGC
AGGAGTCTAAATGAAACAGACCTGGAAGCTCAGGGGCGAGTCCAGAGACACTCAAG
CACACTGGGCTCACTGGCTGGGACCTTGAGCCTCCCGCTCTCCGCGCCGAGTGCCGC
GCCCCCGTCTGTAGCTCGCTGCGCTCGGTACAGAGGAACTACTATGGTTGAAGGGAG
GTGGCAGTTGGGTACCGTCCTCTCCTGCCCCCCGCAGTCGGAGCTGGGGTCTGTCCC
CTCTCGGGGCAGCCTCCAATCTCTGCAACTTTTAAGGCTGAGAACGGCGGCTCCCAG
CTGCTGCACGCTGTCCTGGCCGCCTTTTGCGTTCCTTTTGGCTCCTCCAAGCTCTTCT
GCCCGGTCTGGGCGGGAACCGAGGGCGGAGGCTGCCGTCTTGCGCACCCTCAAGCT
ATCTCTCCGCTGCGGGAAGGCTTCGGACTGTCTGCCTGCTGAACTTCTGGGCGTGAAT
CCCAGCCCCCGCGCTGCGCAAGTTTGCAGCGTCCTTGCTCTCACCGGCGCCTCGGCT
CCTCAGAGTTCGCAG
chr1: 107963936-107966036 (SEQ ID NO. 2)
CCAGGAGCCGACCGGCACCACAGCACCTGAGCAGGGCACTGCAGGAAGGAAAGCG
GAATCTCTAGTGAAACTTCTCATTTCCTGTCGCTGCTGAGAGAGGCTGGACTCGCTCC
TTCTCACATGGCTTAGGAAGAGCTGTAAACGGGAGCTTGCCGGCTGGCCACCAGCTC
AGGGGCCCTGGGGGCGGCCTCCGGGTTCTGCTCCCTGTTCTTCCCTTTGACCAATGTC
ACTCCTGGACAGATAACGGGACCAAACGCCCTTCACTTCCTCGAGTCCTCATGTATGT
CATGGTTCCTCTTTTAGGAAAACATGAGTACAAGACGCAAAGCAAAAGAAGAGACTA
AAGGCAAATTACCCCATCACCTCGTTTCGTCCCTCCCCTTCGTTATTATAGAAGAGCTT
GATCAAATTCCCTTTGTCGCGCCACACACACGCAGAGTAGGTGAAGGGCACCCTAAG
ACAACTTATTTCTTTCCCGCCTCACAGAAAGCCTTTACGAAATCCTCACACCATCTCC
GGACGCAAAGCTTTCGCATTCAGCTTGAGGAGCTAAACCATTTCAAGCCAAGGTAGG
AAACGCCAAAGTGGTGCCGAAGTGGTCCCAAAGCAGAAGGCTGGGAAGCAGGGCA
AGCTCAGCGCACCTAGACGTTTGCATTTACACAAAGAAATTAGCCGCATGATTAATGG
GAGCTGCCGGCTGGAGGCGGGGCGCCCGTGCCGGCCTCCTCACCTGGGACATCTGC
GGCCTCAGGTTGATCTCCTTCAGGTTGATGGAGTGCGCCCGGAGGTTGTTAAGCAGC
TGGCAGAGCAGGACTCCATCGCGGAGGGTCTGCGCAAGGTCGAACACCTGAGCCGA
GTCCCAGGTCACCCGGTGGTTGGTGGGCAGCACCTTGCAATGGATGAGCCACTGCGC
GCACTGCTTCCACGGCTCCATGCCCGACGGCTCCGGGACGCGGCTGGGCCGGGGCG
GGCGGCAAGGATGCGGCCGCCGCCGCCGCCGCCGCGGTTCCTCCGCGCCCCGCCGA
CGCCAACAGCCGCCGGCCCTTTCCCCGCGCGGGATCGAGGGAGCAGGAGCCGCGGC
TGACGGGTCGCGGGCGCCGCGCTAGGCTCGGCTCCGGTCCCGGCCCGGGTGCGCCG
CGACCCGGCCGCCGCTGCAGCGAGTCCCGCGCGCTCTCCGTGCGCCCCGGCCGGCTC
GGCGGCGGCTGCCGCGCACAGGCTTCCGACTCCAGCGCCCGGCCCGCCACTGAGCA
TGCCCAGCACGCCGGCCGGTCTCGCTGCGGTCCGCAAGTCCCCAGACGCGCGGGTG
GGAGCGCGCCGGCGGCCGGGGCTGGGGTCTGTGGCCGAGGGCGGGGCGCGGGGGA
GGGGCCGGCGGAGGGGGGCGGCGGCCAGAAAGGGGATCCCGCGCCCCCGCCTGCA
GCCTTGCGGGGCTCACGCAGCCCCCGGCGTCCTGGGGTCTTCTCTCGGGGCGGCTTC
CCGGCTTTGCGGGGAGTGTGGCTGAATACTGTAATACGATGGGGTCCCCCAGGACCC
CCTAAACAACAAATGCTCAAAGGAGCGACGGATTAATTGGGGCACCCAGACTCCCCA
GAGCAATGAAAAAAGTGCCTAGAGCATCAGTAGAGCACGGCGCTGAAAGTTTTAGA
GATCGTCGCCCCCTCGCCCCTTGCAGCTCTATCCCCTCCATTCTCTATAGCTGATTCCT
CAGCCTATTCCTCCTAGTTGCCCCTAGTGGTGTTTTGGCACCCTCAAAGTGAGTGAGA
GTGCGTGTGTGGAGACGCCTGCGGAAACCGCCCCGATCCCTGAGCCTATTTCCTCGC
GAGGTGATTTTCACTTGGAGCTGGTTTGCCCCTGCACTGTCAGGCTCGGAACTGTTTG
CCGTTGCTGTTCTGGCCCTTTTGCTGACCCCACAAAAACCTGCTTGAGAAAGGCCTG
TGCCACGGTGCTAGACTGCGCATGCGTCGGCGACTGGCGGCCGGGTTTGAGAGCAA
AGCGCGTTAGCCCTGGGCAGCTCCTGCCGGGCTGTTCTGGGATCCTTAGTGAAAGTT
GGAACTTGACCCCAGAACTTTTGCGCAGTGCACAAGCAGTGCATTCGTGTTTCTTA
chr2: 73292434-73292554 (SEQ ID NO. 3)
AGGCCTCCCAGAACGCCTCTGGGAAAGGGGCAGCGCCCAGATCCGGGGAGTAAAGG
TCCGGCGGACCCGGCAGCAAGGCATCGGACCCCGCAGGAAAAGGGGCATCCAGCGG
GGATCTGGA
chr5: 178590240-178590360 (SEQ ID NO. 4)
CTGGGGCAGAGGCTGGGTGCGAGAGGAGCAGGCGGGACAGCCCGAGGCACGAGGT
CCGCCGGGCGCGGGGGTTAGCCTCCGGGTAGCAGCGGATCGCCGCGCACGCCCCCTT
CGCCGCAGC
chr5: 173234839-173234959 (SEQ ID NO. 5)
CACTTGGCCGGTGAAGGCGCGCGGCCCAGCTCTGCGCGCAGCTCTGGGAGGCCCGG
CGCAGCCGCCTCGGGCCCAGCGTAGGCCTCTGGCTTGAAGGCGGCCAGCATGCAGG
AGGAGGGCG
chr20 :62476374-62476494 (SEQ ID NO. 6)
CCCGGCCCCGCTCACCGATGGACACGCGGCGGTGGAACCCCGTGGGCCCCCTCGTTG
CGTGGCCGCACCCGGGGCTCGGTGCAGGGAACCGGCTTCCATAGGGACGGCCGGCT
CGGGTCGC
chr8: 72251358-72251490 (SEQ ID NO. 7)
ACGCTTTCTCCAAACCAAGGGCTGCCCGCAAGGAAACCTCGAGCCGAACCGCGGCC
GGACTTCAGAACCCGTCCCGACCCGCGAACCCCCAGGGTACCTTAAGCGGTTCCTTG
CAGTCCTCCGTGTCGTCCCG
chr2: 236237696-236237816 (SEQ ID NO. 8)
AGGCGGCCGTGCGGCAGAGGTGCAGAGGCGCCAGGCCCTCGGCGCTGAGCAGGTCG
GGGTCGGCGCGGTGCTGCAGCAGCAGGCGGACGCAGGCGGTGTGGCCCCCGAGGCA
GGCCTCGTG
chr6: 105981552-105981672 (SEQ ID NO. 9)
TCGGGTTCTCGGTTCCCGGAGTCCCAGTTCCCGGTTTCCAGTTTCCTTATCGACGCGAT
TGTTCCGTCGGGGTCTTCCAAGGGGATCCGAATGGTGGCCAAGTCCCGCTGGGGAAA
TCCGC
chr20: 63177004-63178804 (SEQ ID NO. 10)
CAGCAGGGCCATCCGCAGGAGGCCGGCGGGACCCTTGCCTTTCTCAGGACTGAGAG
GCTTGGAGGACCGCGGTGTGAGCTGCGCGGCAGAGTGCGTTCTGCAGGCGCCAGAC
AGGAGACGCGAACCCGCGGTCAGGGCCCGAGTGCGGGTGCGTGTCTGCGGGTGCCG
CTGGGCTGCGTGCCGGCGAGGCGTGCGCGTGGCGAGGCGTGCGCGTGGCGGGGTGT
GGCTAGAGGTGTCAATGTGCAGCTGGAGGGGCTGCGCGCGTGGGTGCAGGAGGGCC
ATGTGAGGGGCTGGAGTGTGTGGGAGACAGGCGTTTCCTTGGGTCTCCGTGTGGCCG
CCGGGCGCAGGGCACGGTCGAGGGCGCTCGATGGCTGAGTCCGCGTGAGCCGAGGA
GCGCGCGGGGGGGGACGCGGTGCGAATGCGCGGGAGGCACGAGGCGCGCAGTGCG
TGTGTGCGCGTGTGCGCGGCGCGCGTGCCCCGCAGTTCTCAAGGACACCTCGGGGA
GGCAGCGGCGGGGCCGGTGTCCGGGTGACGTCACCGCGCGCCCCAGTGATAATCGG
CCGGTGCCGGAGCGGAGCGCGGATACGCGCGGAGGCAACGGCGACGGCGGCGGCG
GCGGCGGGCGCGGGGACAGTTGCATCGGGGCCGGGCCGGGCTAGCAGGAGCTGGGC
GCCTGCAGCGTGGACCCCGTGGACACTCGGCTCGCAGCCGGCCTGCGGCGCTCGGG
GACTTGCCTGGCTCCCTTCTCGGGGTTCCCGCGCCCTTCTCCGCCCAGGGCAGCAGC
GCGCGGGGCCCCCGGGAGCCGAAGAGCAGGCGGGAACTGGCGGCGGCGCGGGAGG
CGCAGGGAGCGGAGGCGGCAGCAGCGGCTCCCGCCGGGACTGGTAATTACGCTCGG
GGCCGGGCCGGGGCGAGCCGGGCAAGCGGCCTCTCTGGGTCTCCCCGTCTTTCTCTC
CACGAACAGCTCGAGCGCCTTCTCGCGGGCCCGCTGCGCGCGGAGAGGACGAGCTC
GCTGGGTTGTAAAAAGAGACGAGTTTTCATCTTTGAGCATCGAGATTCGTTCTTTTAA
CCGCATTCGGTGCGCGCTCCTGGGTCGGCACGGGCAGGGCGACGGCAGGGGAAGGC
AGCTGCGGAGGAGCTCGCGCCGCCCAGTCGGAGCGGTTCTGCGCCCCTCGGAGCCC
CGCGGGAGGCGGCCGGGTGCGCACGCGCTCACCACCCCCACCCCCGGAATCCGTCTT
CGCGATTCCCGGGCGCCCCAGCTCCAGGAACGCCCGGAGGGACGCACTTGGGGGCC
CACTCTCTGCCGCGGAAAGGGGAGAAGTGTGGGCTCCTCCGAGTCGGGGGCGGACT
GGGACAGCACAGTCGGCTGAGCGCAGCGCCCCCGCCCTGCCCGCCACGCGGCGAAG
ACGCCTGAGCGTTCGCGCCCCTCGGGCGAGGACCCCACGCAAGCCCGAGCCGGTCC
CGACCCTGGCCCCGACGCTCGCCGCCCGCCCCAGCCCTGAGGGCCCCTCTGCGTGTT
CACAGCGGACCTTGATTTAATGTCTATACAATTAAGGCACGCGGTGAATGCCAAGAGA
GGCGCCTCCGCCGCTCCTTTCTCATGGAAATGGCCCGCGAGCCCGTCCGGCCCAGCG
CCCCTCCCGCGGGAGGAAGGCGAGCCCGGCCCCCGGCGGCCATTCGCGCCGCGGAC
AAATCCGGCGAACAATGCGCCCGCCCAGAGTGCGGCCCAGCTGCCGGGCCGGGGAT
CTGGCCGCGGGACACAAAGGGGCCCGCACGCCTCTGGCGTCGCGGGGCGGGTGGGG
GC
chr2: 100321258-100322771 (SEQ ID NO. 11)
CCACCTTTCACCTTCCCATCCTTAGGAAGCAAAGTGACCCCTAAGCCTAGACAAAGC
TCTCGAAAGCCCAAAGCCTCGGGCCCACCGGCCAGCTCCCCACCCCGCTGCTGGGCC
GGACAGGTGTAGGGGAGGCGGACCCGCCCCGCAGCCGACTCACCCAGCTCCAGGGC
CTGGTCGCACCTGAGCAGCGCGGCCTCCGGCTGCTGCTGGCGCTGCAGGCTCCGCGC
CTGGCCTGCCAGCCTGCGCAGCCGGCACTCGGCCGGGAAGCACTTCTCCAGCAGGC
CGCTCAGCACCACGTTCACGCGCCGCACCTGCGGCCGCGCGGGCCCTGGCTCCACGC
AGCGCTTGCAGACTGTGAGCCCGCAGGGCAGCGTCACCGGCTTATGCAGCAGCCGCC
GGCAGCGCGGGCAGCCGAGCAGGTCGCGGGGCGCGCGGGGCTCCGGGGCCGGCCC
TCCCTCGCCGGGCGCCTCGGGCTCGCCGCCCGGGTTCTCCGCGGACAGCGGCCGGTC
GCGCAGGCCCACGGCGCGCACCAGGCCGCCCGCCAGCTCTTCCAGCTCCTCCGGCC
GCAGCGCCCCGAGCCGCGCGGCGCCGCGGAACGCGCCCAGGGCTTCGGGGAGGCG
GCCGGCGCGGGCCAGCGCGTCCCCCAGCCTCAGGCACAGGCCGCGGTCCGGCTGCG
CCAGCCCGGCTAGCATGGAGCGAAAGAGCTCGGCTGCCATCTCGTAGTCGCCCGCGC
GGAAGGCCTCGTCGCCCTCCTCTAAGCGCTGGGCGATCGGCTCCGCGCGGTCGCAGC
CAGGACACTGGGGCGGCGGCGGCGGCGGGACCGGCTCGGGGCTCATCACCGCGGGG
CTGCGACGACGCGGGTCCGGAGCGAAGGCGCGGAGCAGGGAGGATGCGCTGCTGCT
GGGAACTGGCCGGCGGGAGCGCGGTCTCAGCCCTCGCCAGCAGCCACGCGCGTCTG
GGGGCGGCGCGCTGCGAGCGGCTGAGACCGCGGGCGGGGGCGGGCGCCTGGCTTG
GGCAGCGTCCTCAGCGCGGTGTGGGCGGCGAGCCCCGCAGGGCTGCAATCGTTCCG
GGGTGGGGGCCGGGACAGGCACCGCGGGCGCAATCTGAGCCCCTGCCCACGCGCAG
CGGCCTCTCAGTCCCGCCGGCTTAGGTAACCCAGGTCGCTGCGGTAACGCAGTGACC
GCGCTCCAGGTCCGCGTCTCTTGCGATGCTTCCCCCACTCGCCTGAGGGCTCCTGCGC
GACTGCGCGCGCGTCCTCTGCCTGCCGCCTCCCCGCAGAGGTGCCGGGGCCCTGGGA
GCAGGTGGCCTTGGCCGCGGGCTGCTGGCGCGCCGGCACCGCGGCACCTGCTCTTCC
CCAGAGGCCTGGCCGCCCCCACAACCTGTGGCTCCGCTTAAGCAAGAACCCAGGAA
AAGTCACCAAACGCATCACGCATCTCTAGCTTCGACTTAGGAAATTGTCCTAAATGAC
TGGGGAGGCTGAAGTGGGCACCCAGAGGCCCCGCCTCAGCGAGCTT
chr4:143699944-143701144 (SEQ ID NO. 12)
AGCCTCACAGTCTACGCCCTTGCCCCTGGGGAGAGGGGCCCCCACCGCGTCCACCAA
GCGCCCGTACTTGGGCAGGGGGCCGTCCTCGTGAGGAAGTGGGGTAAGCCGGCACC
TGCGGGTGGCCGTGGCTCCAGACTTCAGGGAGGCGAAGTCCAGCACTCTCCTGTCTA
TGGCGCGGCTCCAGCTTCGCAGCTTCTCCACTACCAAAGGCCTGTTACGCGTCACCA
GCTCCAGCTGGGAGAAGACCAAGTCCACCGCCAGCGTGAAGGGCAGCACCAGAGTG
TGAGTCGGGGCGTCGTAGCGCAGCTGCAGCAGCACCCGGGCGCGTCCGGGGCTGTG
GGAGCCGAAGTGAGTGTACTGGACTTGGCGGGGCCCGAAGGTGCAGGGGAAGCGGC
GCGGGGAGAGCGCGCCCTTGAGCCGCGGCAGGGCGTCCAGTACCGTGACTTCGCAC
CGGTCCCCCGGCTGCACTCCAATCACCAGATCCCGGAGCGGGTCGAGCCAAAGGGA
ACGACCCAGGGGCACCCGGAGTCCAGGGTTGGCAATCAGCACGCTGGGGCCGTCGG
GGCGAGTGCCGTCAAGCGCACCCCGGGCGGGCAGGTAAAGCGCCGGGTCGGGCTCG
GTCCCAAGTGAGGATGCCCGTCCCTGCAGCGCGGGGCGACTCAAGAGCAGGCAGGC
GAGCGCCACAAGGAGCTGCCGGGGCGTCCCAGTCGGGTGCCGAGAAGCCCCCGCCA
TGGCCACGGATGGCTCCTGGCGTTGGGATTCCCGGGGTGGGGTGCCCTGTGCAAAGA
GGGATCTGCTGAGCGGCAGGTGCAGGCAGTGGAAGCAGTAGCTGCTGTCCAGTCGG
TAGCCGACTTGCGGATCCAGCAAGAGCCAGCGGCTGCGCTTCGGCTGCTGCAGGTAA
CGGCAGCGGGGGAAGGGGCTCTGCCCACTTCCTGCTCAGCCCCGGTCGCAAGTCTCT
CTCTGCTGGCTTCTGGGGACCCCAGATACGCGCCCAGCGCGGCGAGACTTAGCGAGG
GTGCAGCGCTGTCCCCTCCGCTCCTGGGCGCTTCACCCAGCCTACCTTACACACCTTC
TCGCCGGGAGCCGTGGCCGCCGCACTGCTGCCCGCGCTGCCAGACTCCGACCAGCT
GTCTGGATACTCTCTTCCCCAGGTGCCACAAAGGGATTGTCCCTCAGGGTTGGGAGA
GAGACGGTGACTGTA

### OTHER EMBODIMENTS

While we have described a number of embodiments, it is apparent that our basic disclosure and examples may provide other embodiments that utilize or are encompassed by the compositions and methods described herein. Therefore, it will be appreciated that the scope of is to be defined by that which may be understood from the disclosure and the appended claims rather than by the specific embodiments that have been represented by way of example. All references cited herein are hereby incorporated by reference.

1.-127. (canceled)

128. A method of detecting methylation status of one or more markers, the method comprising:
detecting a methylation status for each of one or more markers identified in deoxyribonucleic acid (DNA) from a sample, wherein each of the one or more markers is a methylation locus comprising at least a single differentially methylated region (DMR) or a portion of a DMR selected from the group consisting of SEQ ID NO. 10 (chr20: 63177004-63178804) and SEQ ID NO. 11 (chr2: 100321258-100322771).

129. The method of claim 128, wherein at least one of the one or more markers comprises a methylation locus comprising at least a portion of SEQ ID NO. 11 (chr2: 100321258-100322771).

130. The method of claim 128, wherein at least one of the one or more markers comprises a methylation locus comprising at least a portion of SEQ ID NO. 10 (chr20: 63177004-63178804).

131. The method of claim 128, wherein a first of the one or more markers is a methylation locus comprising at least a portion of SEQ ID NO. 10 (chr20: 63177004-63178804) and a second of the one or more markers is a methylation locus comprising at least a portion SEQ ID NO. 11 (chr2: 100321258-100322771).

132. The method of claim 128, wherein detecting the methylation status comprises determining whether at least one methylation site within at least one of the one or more markers is hypermethylated or hypomethylated.

133. The method of claim 128, wherein the sample comprises DNA isolated from blood or plasma of a human subject.

134. The method of claim 133, wherein the subject is susceptible to colorectal cancer and/or advanced adenoma.

135. The method of claim 133, wherein the human subject is susceptible to stage III or stage IV colorectal cancer.

136. The method of claim 133, wherein the human subject is susceptible to early stage colorectal cancer.

137. The method of claim 128, wherein each methylation locus is equal to or less than 2200 bp in length.

138. The method of claim 128, wherein the sample is a member selected from the group consisting of a tissue sample, a blood sample, a stool sample, and a blood product sample.

139. The method of claim 133, wherein the method comprises isolating DNA from at least 3 mL of plasma from the human subject.

140. The method of claim 128, wherein the sample comprises at least 8 ng of DNA.

141. A method of detecting methylation statuses of one or more markers, the method comprising:
converting unmethylated cytosines of a plurality of DNA fragments in a sample into uracils to generate a plurality of converted DNA fragments;
sequencing the plurality of converted DNA fragments to generate a plurality of sequence reads, wherein each sequence read corresponds to a converted DNA fragment; and
detecting a methylation status for each of one or more markers identified in the sequence reads, wherein each of the one or more markers is a methylation locus comprising at least a single differentially methylated region (DMR) or a portion of a DMR selected from the group consisting of SEQ ID NO. 10 (chr20: 63177004-63178804) and SEQ ID NO. 11 (chr2: 100321258-100322771).

142. The method of claim 141, wherein converting the unmethylated cytosines of a plurality of DNA fragments in the sample into uracils comprises subjecting the plurality of DNA fragments to an enzymatic treatment.

143. The method of claim 141, wherein the method comprises adding one or more control DNA molecules to the sample, wherein the sequence, number of methylated bases, and number of unmethylated bases of the control DNA molecules had been determined prior to the addition of the one or more control DNA molecule(s) to the sample.

144. The method of claim 141, wherein the method comprises determining the number of unmethylated cytosines of the control DNA molecules that were converted into uracils.

145. The method of claim 141, wherein the method comprises attaching adapters to the plurality of DNA fragments.

146. The method of claim 145, wherein the adapter sequence is attached to the plurality of DNA fragments prior to conversion.

147. The method of claim 146, wherein the method comprises amplifying the plurality of converted DNA fragments.

148. The method of claim 147, wherein the method comprises amplifying the plurality of converted DNA fragments after attaching adapters to the plurality of DNA fragments.

149. The method of claim 146, wherein the method comprises performing one or more quality control checks to determine the concentration and/or the ratios of fragments lengths of the amplified DNA fragments.

150. The method of claim 141, wherein the method comprises using one or more capture baits that enrich for a target region to capture one or more corresponding methylation locus/loci.

151. The method of claim 150, wherein the capture baits comprise at least one capture probe that targets a fully methylated methylation locus.

152. The method of claim 141, wherein the method comprises capturing a subset of the DNA fragments using the one or more capture baits.

153. The method of claim 152, wherein the method comprises binding the captured subset of the DNA fragments to a substrate.

154. The method of claim 141, wherein the method comprises binding the captured subset of the DNA fragments to the substrate after amplification of the converted DNA fragments.

155. The method of claim 141, wherein the method comprises sequencing the plurality of converted DNA fragments at a read depth of at least 50×.

156. The method of claim 141, wherein the method comprises mapping a subset of the plurality of sequence reads to a region of interest in a reference genome comprising at least one of the one or more markers.

157. The method of claim 156, wherein the region of interest in the reference genome comprises at least 100 base pairs upstream and/or downstream of the at least one of the one or more markers.

158. The method of claim 141, wherein the method further comprises deduplicating the plurality of sequence reads generated from the plurality of converted DNA fragments.

159. The method of claim 158, wherein the method comprises deduplicating the plurality of sequence reads based on:
(i) the start position of the sequence reads (i.e., the 5′ end coordinate); and/or
(ii) the end position of the sequence reads (i.e., the 3′ end coordinate); and/or
(iii) the methylation level of the sequence reads.

160. The method of claim 141, wherein the method comprises removing, from the plurality of sequence reads, one or more poor-quality reads that failed one or more quality check criteria.

161. The method of claim 141, wherein the methylation status is a read-wise methylation value.

162. A method of detecting methylation status of one or more markers, the method comprising:
converting unmethylated cytosines of a plurality of DNA fragments in a sample into uracils to generate a plurality of converted DNA fragments;
sequencing the plurality of converted DNA fragments to generate a plurality of sequence reads, wherein each sequence read corresponds to a converted DNA fragment; and
detecting a methylation status for one or more markers identified in the sequence reads,
wherein each of the one or more markers is a methylation locus comprising at least a portion of a gene,
wherein a first of the one or more markers is a methylation locus comprising at least a portion of gene LONRF2 and a second of the one or more markers is a methylation locus comprising at least a portion of gene MIR124-3.


SEQUENCE LISTING (12 sequences)

SEQ ID NO: 1 - DNA, 1320 bp, Homo sapiens
  source 1..1320: mol_type = genomic DNA
ggacagccttccctctctgcccacttccgacgccttcttctcgggcatcaggcggatcctcagtcgcccttcgccttggcgaatccaccaactgaacagctcgctgagattgaactggagcagccccacagccgcctccccggggggcccgacgcaaccctccaagatcgcctcctcgcccagctccagcaccaactgcttggcacgccggagcttgcgcacggagccgcccttcagcaccctggacagcgtccgggcctctgccggggctggtgaaccggcggtccaggagacccccggcgccggccccagcaacctgagcagcggggcgcagtccagagctagcgagccgcgggcctcgggcctgccagccttcagctccgaggaggatggtggcagcagtaggtcccgggcgtagacacggaagagcgagggcaccacgaagtcaactgccagactcttcctctgcaggcgcgagtagctgagtggctcccggggctgcagcggcggccccgcagctggggagcccgcgcgctggccggtccccatcccggagcccacagctgccgtggaaagcagcagcggcaggagccacaggagcccgatggctcccatcccgccggaggaggccgtttacactgctctccgggcccagcctcacccttcgctctccccgagatgggaagaggctctgaacagtccttggtacccagcggctccttccacctgatctccagaggactgtgcgtgcgcgcaagtctcttgctttcccccaactgcacggaggcgagcaggagtctaaatgaaacagacctggaagctcaggggcgagtccagagacactcaagcacactgggctcactggctgggaccttgagcctcccgctctccgcgccgagtgccgcgcccccgtctgtagctcgctgcgctcggtacagaggaactactatggttgaagggaggtggcagttgggtaccgtcctctcctgccccccgcagtcggagctggggtctgtcccctctcggggcagcctccaatctctgcaacttttaaggctgagaacggcggctcccagctgctgcacgctgtcctggccgccttttgcgttccttttggctcctccaagctcttctgcccggtctgggcgggaaccgagggcggaggctgccgtcttgcgcaccctcaagctatctctccgctgcgggaaggcttcggactgtctgcctgctgaacttctgggcgtgaatcccagcccccgcgctgcgcaagtttgcagcgtccttgctctcaccggcgcctcggctcctcagagttcgcag

SEQ ID NO: 2 - DNA, 2101 bp, Homo sapiens
  source 1..2101: mol_type = genomic DNA
ccaggagccgaccggcaccacagcacctgagcagggcactgcaggaaggaaagcggaatctctagtgaaacttctcatttcctgtcgctgctgagagaggctggactcgctccttctcacatggcttaggaagagctgtaaacgggagcttgccggctggccaccagctcaggggccctgggggcggcctccgggttctgctccctgttcttccctttgaccaatgtcactcctggacagataacgggaccaaacgcccttcacttcctcgagtcctcatgtatgtcatggttcctcttttaggaaaacatgagtacaagacgcaaagcaaaagaagagactaaaggcaaattaccccatcacctcgtttcgtccctccccttcgttattatagaagagcttgatcaaattccctttgtcgcgccacacacacgcagagtaggtgaagggcaccctaagacaacttatttctttcccgcctcacagaaagcctttacgaaatcctcacaccatctccggacgcaaagctttcgcattcagcttgaggagctaaaccatttcaagccaaggtaggaaacgccaaagtggtgccgaagtggtcccaaagcagaaggctgggaagcagggcaagctcagcgcacctagacgtttgcatttacacaaagaaattagccgcatgattaatgggagctgccggctggaggcggggcgcccgtgccggcctcctcacctgggacatctgcggcctcaggttgatctccttcaggttgatggagtgcgcccggaggttgttaagcagctggcagagcaggactccatcgcggagggtctgcgcaaggtcgaacacctgagccgagtcccaggtcacccggtggttggtgggcagcaccttgcaatggatgagccactgcgcgcactgcttccacggctccatgcccgacggctccgggacgcggctgggccggggcgggcggcaaggatgcggccgccgccgccgccgccgcggttcctccgcgccccgccgacgccaacagccgccggccctttccccgcgcgggatcgagggagcaggagccgcggctgacgggtcgcgggcgccgcgctaggctcggctccggtcccggcccgggtgcgccgcgacccggccgccgctgcagcgagtcccgcgcgctctccgtgcgccccggccggctcggcggcggctgccgcgcacaggcttccgactccagcgcccggcccgccactgagcatgcccagcacgccggccggtctcgctgcggtccgcaagtccccagacgcgcgggtgggagcgcgccggcggccggggctggggtctgtggccgagggcggggcgcgggggaggggccggcggaggggggcggcggccagaaaggggatcccgcgcccccgcctgcagccttgcggggctcacgcagcccccggcgtcctggggtcttctctcggggcggcttcccggctttgcggggagtgtggctgaatactgtaatacgatggggtcccccaggaccccctaaacaacaaatgctcaaaggagcgacggattaattggggcacccagactccccagagcaatgaaaaaagtgcctagagcatcagtagagcacggcgctgaaagttttagagatcgtcgccccctcgccccttgcagctctatcccctccattctctatagctgattcctcagcctattcctcctagttgcccctagtggtgttttggcaccctcaaagtgagtgagagtgcgtgtgtggagacgcctgcggaaaccgccccgatccctgagcctatttcctcgcgaggtgattttcacttggagctggtttgcccctgcactgtcaggctcggaactgtttgccgttgctgttctggcccttttgctgaccccacaaaaacctgcttgagaaaggcctgtgccacggtgctagactgcgcatgcgtcggcgactggcggccgggtttgagagcaaagcgcgttagccctgggcagctcctgccgggctgttctgggatccttagtgaaagttggaacttgaccccagaacttttgcgcagtgcacaagcagtgcattcgtgtttctta

SEQ ID NO: 3 - DNA, 121 bp, Homo sapiens
  source 1..121: mol_type = genomic DNA
aggcctcccagaacgcctctgggaaaggggcagcgcccagatccggggagtaaaggtccggcggacccggcagcaaggcatcggaccccgcaggaaaaggggcatccagcggggatctgga

SEQ ID NO: 4 - DNA, 121 bp, Homo sapiens
  source 1..121: mol_type = genomic DNA
ctggggcagaggctgggtgcgagaggagcaggcgggacagcccgaggcacgaggtccgccgggcgcgggggttagcctccgggtagcagcggatcgccgcgcacgcccccttcgccgcagc

SEQ ID NO: 5 - DNA, 121 bp, Homo sapiens
  source 1..121: mol_type = genomic DNA
cacttggccggtgaaggcgcgcggcccagctctgcgcgcagctctgggaggcccggcgcagccgcctcgggcccagcgtaggcctctggcttgaaggcggccagcatgcaggaggagggcg

SEQ ID NO: 6 - DNA, 121 bp, Homo sapiens
  source 1..121: mol_type = genomic DNA
cccggccccgctcaccgatggacacgcggcggtggaaccccgtgggccccctcgttgcgtggccgcacccggggctcggtgcagggaaccggcttccatagggacggccggctcgggtcgc

SEQ ID NO: 7 - DNA, 133 bp, Homo sapiens
  source 1..133: mol_type = genomic DNA
acgctttctccaaaccaagggctgcccgcaaggaaacctcgagccgaaccgcggccggacttcagaacccgtcccgacccgcgaacccccagggtaccttaagcggttccttgcagtcctccgtgtcgtcccg

SEQ ID NO: 8 - DNA, 121 bp, Homo sapiens
  source 1..121: mol_type = genomic DNA
aggcggccgtgcggcagaggtgcagaggcgccaggccctcggcgctgagcaggtcggggtcggcgcggtgctgcagcagcaggcggacgcaggcggtgtggcccccgaggcaggcctcgtg

SEQ ID NO: 9 - DNA, 121 bp, Homo sapiens
  source 1..121: mol_type = genomic DNA
tcgggttctcggttcccggagtcccagttcccggtttccagtttccttatcgacgcgattgttccgtcggggtcttccaaggggatccgaatggtggccaagtcccgctggggaaatccgc

SEQ ID NO: 10 - DNA, 1801 bp, Homo sapiens
  source 1..1801: mol_type = genomic DNA
cagcagggccatccgcaggaggccggcgggacccttgcctttctcaggactgagaggcttggaggaccgcggtgtgagctgcgcggcagagtgcgttctgcaggcgccagacaggagacgcgaacccgcggtcagggcccgagtgcgggtgcgtgtctgcgggtgccgctgggctgcgtgccggcgaggcgtgcgcgtggcgaggcgtgcgcgtggcggggtgtggctagaggtgtcaatgtgcagctggaggggctgcgcgcgtgggtgcaggagggccatgtgaggggctggagtgtgtgggagacaggcgtttccttgggtctccgtgtggccgccgggcgcagggcacggtcgagggcgctcgatggctgagtccgcgtgagccgaggagcgcgcggggcgggacgcggtgcgaatgcgcgggaggcacgaggcgcgcagtgcgtgtgtgcgcgtgtgcgcggcgcgcgtgccccgcagttctcaaggacacctcggggaggcagcggcggggccggtgtccgggtgacgtcaccgcgcgccccagtgataatcggccggtgccggagcggagcgcggatacgcgcggaggcaacggcgacggcggcggcggcggcgggcgcggggacagttgcatcggggccgggccgggctagcaggagctgggcgcctgcagcgtggaccccgtggacactcggctcgcagccggcctgcggcgctcggggacttgcctggctcccttctcggggttcccgcgcccttctccgcccagggcagcagcgcgcggggcccccgggagccgaagagcaggcgggaactggcggcggcgcgggaggcgcagggagcggaggcggcagcagcggctcccgccgggactggtaattacgctcggggccgggccggggcgagccgggcaagcggcctctctgggtctccccgtctttctctccacgaacagctcgagcgccttctcgcgggcccgctgcgcgcggagaggacgagctcgctgggttgtaaaaagagacgagttttcatctttgagcatcgagattcgttcttttaaccgcattcggtgcgcgctcctgggtcggcacgggcagggcgacggcaggggaaggcagctgcggaggagctcgcgccgcccagtcggagcggttctgcgcccctcggagccccgcgggaggcggccgggtgcgcacgcgctcaccacccccacccccggaatccgtcttcgcgattcccgggcgccccagctccaggaacgcccggagggacgcacttgggggcccactctctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctctgcgtgttcacagcggaccttgatttaatgtctatacaattaaggcacgcggtgaatgccaagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgtcgcggggcgggtgggggc

SEQ ID NO: 11 - DNA, 1514 bp, Homo sapiens
  source 1..1514: mol_type = genomic DNA
ccacctttcaccttcccatccttaggaagcaaagtgacccctaagcctagacaaagctctcgaaagcccaaagcctcgggcccaccggccagctccccaccccgctgctgggccggacaggtgtaggggaggcggacccgccccgcagccgactcacccagctccagggcctggtcgcacctgagcagcgcggcctccggctgctgctggcgctgcaggctccgcgcctggcctgccagcctgcgcagccggcactcggccgggaagcacttctccagcaggccgctcagcaccacgttcacgcgccgcacctgcggccgcgcgggccctggctccacgcagcgcttgcagactgtgagcccgcagggcagcgtcaccggcttatgcagcagccgccggcagcgcgggcagccgagcaggtcgcggggcgcgcggggctccggggccggccctccctcgccgggcgcctcgggctcgccgcccgggttctccgcggacagcggccggtcgcgcaggcccacggcgcgcaccaggccgcccgccagctcttccagctcctccggccgcagcgccccgagccgcgcggcgccgcggaacgcgcccagggcttcggggaggcggccggcgcgggccagcgcgtcccccagcctcaggcacaggccgcggtccggctgcgccagcccggctagcatggagcgaaagagctcggctgccatctcgtagtcgcccgcgcggaaggcctcgtcgccctcctctaagcgctgggcgatcggctccgcgcggtcgcagccaggacactggggcggcggcggcggcgggaccggctcggggctcatcaccgcggggctgcgacgacgcgggtccggagcgaaggcgcggagcagggaggatgcgctgctgctgggaactggccggcgggagcgcggtctcagccctcgccagcagccacgcgcgtctgggggcggcgcgctgcgagcggctgagaccgcgggcgggggcgggcgcctggcttgggcagcgtcctcagcgcggtgtgggcggcgagccccgcagggctgcaatcgttccggggtgggggccgggacaggcaccgcgggcgcaatctgagcccctgcccacgcgcagcggcctctcagtcccgccggcttaggtaacccaggtcgctgcggtaacgcagtgaccgcgctccaggtccgcgtctcttgcgatgcttcccccactcgcctgagggctcctgcgcgactgcgcgcgcgtcctctgcctgccgcctccccgcagaggtgccggggccctgggagcaggtggccttggccgcgggctgctggcgcgccggcaccgcggcacctgctcttccccagaggcctggccgcccccacaacctgtggctccgcttaagcaagaacccaggaaaagtcaccaaacgcatcacgcatctctagcttcgacttaggaaattgtcctaaatgactggggaggctgaagtgggcacccagaggccccgcctcagcgagctt

SEQ ID NO: 12 - DNA, 1201 bp, Homo sapiens
  source 1..1201: mol_type = genomic DNA
agcctcacagtctacgcccttgcccctggggagaggggcccccaccgcgtccaccaagcgcccgtacttgggcagggggccgtcctcgtgaggaagtggggtaagccggcacctgcgggtggccgtggctccagacttcagggaggcgaagtccagcactctcctgtctatggcgcggctccagcttcgcagcttctccactaccaaaggcctgttacgcgtcaccagctccagctgggagaagaccaagtccaccgccagcgtgaagggcagcaccagagtgtgagtcggggcgtcgtagcgcagctgcagcagcacccgggcgcgtccggggctgtgggagccgaagtgagtgtactggacttggcggggcccgaaggtgcaggggaagcggcgcggggagagcgcgcccttgagccgcggcagggcgtccagtaccgtgacttcgcaccggtcccccggctgcactccaatcaccagatcccggagcgggtcgagccaaagggaacgacccaggggcacccggagtccagggttggcaatcagcacgctggggccgtcggggcgagtgccgtcaagcgcaccccgggcgggcaggtaaagcgccgggtcgggctcggtcccaagtgaggatgcccgtccctgcagcgcggggcgactcaagagcaggcaggcgagcgccacaaggagctgccggggcgtcccagtcgggtgccgagaagcccccgccatggccacggatggctcctggcgttgggattcccggggtggggtgccctgtgcaaagagggatctgctgagcggcaggtgcaggcagtggaagcagtagctgctgtccagtcggtagccgacttgcggatccagcaagagccagcggctgcgcttcggctgctgcaggtaacggcagcgggggaaggggctctgcccacttcctgctcagccccggtcgcaagtctctctctgctggcttctggggaccccagatacgcgcccagcgcggcgagacttagcgagggtgcagcgctgtcccctccgctcctgggcgcttcacccagcctaccttacacaccttctcgccgggagccgtggccgccgcactgctgcccgcgctgccagactccgaccagctgtctggatactctcttccccaggtgccacaaagggattgtccctcagggttgggagagagacggtgactgta